(19) United States
(12) Patent Application Publication
(10) Pub. No.: US 2021/0047427 A1
(43) Pub. Date: Feb. 18, 2021

(54) SUBCUTANEOUS DOSING OF ANTI-CD38 ANTIBODIES

(71) Applicant: TAKEDA PHARMACEUTICAL COMPANY LIMITED, OSAKA (JP)

(72) Inventors: Eric Fedyk, San Diego, CA (US); Michael Hanley, San Diego, CA (US); Antonio Palumbo, San Diego, CA (US)

(21) Appl. No.: 17/041,783

(22) PCT Filed: Mar. 27, 2019
(86) PCT No.: PCT/IB2019/000314

§ 371 Date: Sep. 25, 2020

Related U.S. Application Data
(60) Provisional application No. 62/649,489, filed on Mar. 28, 2018.

(51) Int. Cl. C07K 16/28; C07K 16/30

(52) U.S. Cl. CPC C07K 16/2896; A61K 9/0019; C07K 2317/565; C07K 16/3061

(57) ABSTRACT

Methods of administering isolated anti-CD38 antibodies subcutaneously are disclosed. The methods provide an effective treatment for autoimmune diseases and cancers, including hematologic diseases. Also disclosed are unit dosage forms for the anti-CD38 antibodies.

### CROSS REFERENCE TO RELATED APPLICATIONS

This application claims the benefit of U.S. Provisional Application No. 62/649,489, filed on Mar. 28, 2018, which is hereby incorporated by reference in its entirety.

### FIELD OF THE INVENTION

Methods and compositions for administering isolated anti-CD38 antibodies via subcutaneous (SC) administration are disclosed.

### BACKGROUND OF THE INVENTION

CD38, also known as cyclic ADP ribose hydrolase, is a type II transmembrane glycoprotein with a long C-terminal extracellular domain and a short N-terminal cytoplasmic domain. CD38 is a member of a group of related membrane bound or soluble enzymes that comprises CD157 and Aplysia ADPR cyclase. This family of enzymes has the unique capacity to convert NAD to cyclic ADP ribose or nicotinic acid-adenine dinucleotide phosphate. CD38 is involved in Ca²⁺ mobilization and in signal transduction through tyrosine phosphorylation of numerous signaling molecules, including phospholipase Cy, ZAP-70, syk, and c-cbl. Based on these observations, CD38 is an important signaling molecule in the maturation and activation of lymphoid cells during their normal development. Among hematopoietic cells, an assortment of functional effects have been ascribed to CD38-mediated signalling, including lymphocyte proliferation, cytokine release, regulation of B and myeloid cell development and survival, and induction of dendritic cell (DC) maturation.

CD38 is expressed in immature hematopoietic cells, down regulated in mature hematopoietic cells, and re-expressed at high levels in activated lymphocytes and plasma cells. For example, high CD38 expression is seen in activated B cells, plasma cells, activated CD4+ T cells, activated CD8+ T cells, NK cells, NKT cells, mature DCs and activated monocytes (see, e.g., U.S. Pat. No. 8,362,211).

The presence of autoantibodies to CD38 has been associated with a number of diseases, including diabetes, chronic autoimmune thyroiditis and Graves' disease (see Antonelli et al. (2001) Clin. Exp. Immunol. 126: 426-431; Mallone et al. (2001) Diabetes 50: 752 and Antonelli et al. (2004) J. Endocrinol. Invest. 27: 695-707).

Increased expression of CD38 has been documented in a variety of diseases, including autoimmune diseases and cancers. Such diseases include systemic lupus erythematosus (SLE), rheumatoid arthritis (RA), inflammatory bowel disease (IBD) and ulcerative colitis (UC). In patients with RA, plasma cells are increased in the joint tissue compared to controls. In patients with SLE, plasmablasts are increased in the peripheral blood in patients with more active disease. Current CD20-based B cell depleting therapies such as rituximab effectively deplete CD20+ B cells, but cannot directly and effectively deplete plasma cells or plasmablasts because they do not express CD20. Consistent with this idea, patients with RA or SLE with high levels of plasma cells or plasmablasts are unlikely to gain substantial clinical benefit from CD20-based therapies. Thus, therapeutics that target CD38, which is highly expressed on plasma cells and plasmablasts as well as NK cells and activated T cells, may provide an effective treatment for RA and SLE as well as other diseases characterized by CD-38 expression.

In particular, increased expression of CD38 has been documented in a variety of diseases of hematopoietic origin, as well as cell-lines derived therefrom, and has been described as a negative prognostic marker in hematologic cancers. Such diseases include, but are not limited to, multiple myeloma (MM), chronic lymphoblastic leukemia, B-cell chronic lymphocytic leukemia (B-CLL), including B-cell acute lymphocytic leukemia, B and T acute lymphocytic leukemia (ALL), acute lymphoblastic leukemia, Waldenstrom macroglobulinemia, mantle-cell lymphoma, pro-lymphocytic/myelocytic leukemia, acute myeloid leukemia (AML), chronic myeloid leukemia (CML), follicular lymphoma, NK-cell leukemia, plasma-cell leukemia, non-Hodgkin lymphoma (NHL), Burkitt lymphoma (BL), T cell lymphoma (TCL), hairy cell leukemia (HCL), and Hodgkin Lymphoma (HL). Furthermore, CD38 expression is a prognostic indicator for patients with conditions such as, for example, B-CLL (Drig et al. (2002) Leukemia 16: 30-35; and Morabito et al. (2001) Leukemia Res. 25: 927-932) and acute myelogenous leukemia (Keyhani et al. (1999) Leukemia Res. 24: 153-159). CD38 therefore provides a useful target in the treatment of diseases of the hematopoietic system.

Several anti-CD38 antibodies are in clinical trials for the treatment of CD38-associated cancers. However, the prior art therapeutic antibodies to CD38 all bind to red blood cells (RBCs) and platelets, leading to higher required dosing due to the sink of unproductive binding to RBCs. Although CD38 is expressed on RBCs at a level that is approximately 1000-fold lower than that on myeloma cells (deWeers et al. (2011) J. Immunol. 186(3):1840-1848), there are approximately 36,000 RBCs for each myeloma cell in the blood of MM (multiple myeloma) patients with active disease (Witzig et al. (1993) Cancer 72(1): 108-113). As such, there are 36-fold more CD38 molecules expressed on RBCs than on tumor cells. Thus, current treatments using anti-CD38 antibodies require intravenous administration due to the high doses needed for efficacy beyond the RBC binding. For example, daratumumab (anti-CD38 IgG1 mAb; DARZALEX® FDA approved and commercially available from Janssen Oncology), requires a very high dose (≥16 mg/kg) and an intensive regime (weekly 8×, bi-weekly 8×, then monthly) for optimal anti-tumor activity (Xu et al. (2017) Clin. Pharmacol. Ther. 101(6): 721-724).

Accordingly, treatments using anti-CD38 antibodies that bind to RBCs are currently focused on intravenous administration because of the high volume of antibody required to achieve therapeutic efficacy, as such large volumes are not suitable for subcutaneous administration. For example, daratumumab cannot be administered in a low volume because target saturation requires ≥16 mg/kg (e.g., approximately 1120 mg per 70 kg patient). The highest known subcutaneous formulation concentration is 200 mg/ml (Cimzia®, also referred to as certolizumab pegol). Using the highest known subcutaneous formulation concentration of 200 mg/ml, daratumumab would have a minimum projected injection volume of 5.6-11.2 mL, a very large volume for subcutaneous administration. Given this large volume and concentration limits, Daratumumab must be administered subcutaneously in a 15 ml volume together with hyaluronidase to aid in dispersion and absorption.

Another anti-CD-38 antibody, isatuximab (commercially available from Sanofi Genzyme and currently in Phase 3 clinical trials), is administered at 10 mg/kg and 20 mg/kg in Phase 3 trials, which corresponds to 700-1400 mg per 70 kg patient. Again, using the highest known subcutaneous formulation concentration of 200 mg/mL, isatuximab would have a projected injection volume of 3.5-14 mL.

In addition to the higher doses and volumes required of the prior art anti-CD38 antibodies presently in the clinic, their targeting of RBCs and platelets may cause serious side effects such as, for example, hemolytic anemia, a condition in which RBCs are destroyed more quickly than they can be replaced. In an open-label, single-arm study, isatuximab was administered intravenously to 97 total patients at 3 mg/kg every 2 weeks (Q2W; n=23), 10 mg/kg Q2W for 2 cycles followed by Q4W (n=25), 10 mg/kg Q2W (n=24), and 20 mg/kg every week for 4 doses (1 cycle) followed by Q2W (n=25). The most common severe (grade 3/4) adverse event was anemia, which affected 24%) of patients (see http://www.onclive.com/conference-coverage/asco-2016/isatuximab-monotherapy-effective-for-heavily-pretreated-myeloma, the 2016 ASCO Annual Meeting as well as Richter et al. (2016) J. Clin. Oncol. 34 (suppl): abstr 8005). In addition to severe anemia, thrombocytopenia and neutropenia are also common severe adverse events (22.9%, 18.4%, and 18.4% respectively; Dimopoulos et al. (2018) Blood 132 (suppl. 1): ASH abstract 155/oral presentation). Likewise, low blood cell counts (WBCs, RBCs, and platelets), anemia, and thrombocytopenia are well-known serious adverse reactions to daratumumab. In one daratumumab study, 45% of all patients experienced anemia (19% of which were grade 3) and 48% of patients experienced thrombocytopenia (10% of which were grade 3 and 8% of which were grade 4) (see, for example, Darzalex (daratumumab) prescribing information. Horsham, Pa.: Janssen Biotech, Inc. 2018; as well as the review article Costello (2017) Ther. Adv. Hematol. 8(1): 28-37). Further, intravenous administration of therapeutic monoclonal antibodies can lead to severe infusion related reactions (IRRs). Common IRRs include but are not limited to nasal congestion, cough, allergic rhinitis, throat irritation, dyspnea, chills, nausea, hypoxia, hypertension etc. (Usmani et al. (2016) Blood 128(1): 37-44). With daratumumab, 48% of patients experience an IRR with the first dose of treatment (Usmani et al. (2016) Blood 128(1): 37-44) with 3% of those being severe (Darzalex (daratumumab) prescribing information. Horsham, Pa.: Janssen Biotech, Inc 2018). Similarly, IRRs have been reported in 40.4% of patients receiving isatuximab with 4.6% reported as severe (Dimopoulos et al. (2018) Blood 132: (suppl 1) ASH abstract 155/oral presentation). Thus, patients being treated with isatuximab or daratumumab must be carefully monitored for these-life threatening and other serious side effects.

AB79 is a fully human immunoglobulin IgG1 monoclonal antibody that binds specifically to CD38 with high affinity (Kd=6.1×10⁻¹⁰ M). AB79 inhibits the growth of tumor cells expressing CD38 by cell depletion via antibody dependent cellular cytotoxicity (ADCC) and complement dependent cytotoxicity (CDC). AB79 also reduces the level of plasma cells and plasmablasts in blood isolated from healthy subjects and systemic lupus erythematosus (SLE) patients (PCT Application No. PCT/US2017/042128). In healthy cynomolgus monkeys, the efficiency of depletion for each cell type correlated positively with level of CD38 expression and AB79 dose level (PCT Application No. PCT/US2017/042128). Furthermore, AB79 demonstrated anti-inflammatory and disease modifying activity in a monkey model of rheumatoid arthritis (U.S. Pat. No. 8,362,211).

Given that many CD38 antibodies in the clinic bind RBCs and are therefore not suitable for subcutaneous administration and which possess dangerous side effects, there remains a need in the art for subcutaneous antibody formulations that are safer, more convenient, and more effective for treating diseases in which binding to CD38 is indicated, such as autoimmune diseases and hematologic forms of cancer.

### SUMMARY OF THE INVENTION

Provided herein are methods for treating diseases in which binding to CD38 is indicated such as, for example, autoimmune diseases and hematological cancers comprising subcutaneously administering isolated anti-CD38 antibodies.

In one aspect, the invention provides a method for treating a disease in which binding to CD38 is indicated in a subject, the method comprising the step of subcutaneously administering to a subject having a disease in which binding to CD38 is indicated a therapeutically effective amount of an isolated human anti-CD38 antibody sufficient to treat the disease, wherein the anti-CD38 antibody comprises a variable heavy (VH) chain region comprising a CDR1 having the amino acid sequence of SEQ ID NO:3, a CDR2 having the amino acid sequence of SEQ ID NO:4, and a CDR3 having the amino acid sequence of SEQ ID NO:5 or variants of those sequences having up to three amino acid changes; and a variable light (VL) chain region comprising a CDR1 having the amino acid sequence of SEQ ID NO:6, a CDR2 having the amino acid sequence of SEQ ID NO:7 and a CDR3 having the amino acid sequence of SEQ ID NO:8 or variants of those sequences having up to three amino acid changes, and wherein the anti-CD38 antibody is administered in a dosage of from 45 to 1,800 milligrams.

In another aspect, the invention provides a method for treating a disease in which binding to CD38 is indicated in a subject, the method comprising the step of subcutaneously administering to a subject having a disease in which binding to CD38 is indicated a therapeutically effective amount of an isolated human anti-CD38 antibody sufficient to treat the disease, wherein the anti-CD38 antibody comprises a VH chain region comprising a CDR1 having the amino acid sequence of SEQ ID NO:3, a CDR2 having the amino acid sequence of SEQ ID NO:4, and a CDR3 having the amino acid sequence of SEQ ID NO:5 or variants of those sequences having up to three amino acid substitutions; and a VL chain region comprising a CDR1 having the amino acid sequence of SEQ ID NO:6, a CDR2 having the amino acid sequence of SEQ ID NO:7 and a CDR3 having the amino acid sequence of SEQ ID NO:8 or variants of those sequences having up to three amino acid substitutions, wherein the anti-CD38 antibody is administered at a dosage of from 45 to 1,800 milligrams.

In another aspect, the invention provides a method for treating a disease in which binding to CD38 is indicated in a subject, the method comprising the step of subcutaneously administering to a subject having a disease in which binding to CD38 is indicated a therapeutically effective amount of an isolated human anti-CD38 antibody sufficient to treat the disease, wherein the anti-CD38 antibody comprises a VH chain region comprising a CDR1 having the amino acid sequence of SEQ ID NO:3, a CDR2 having the amino acid sequence of SEQ ID NO:4, and a CDR3 having the amino acid sequence of SEQ ID NO:5; and a VL chain region comprising a CDR1 having the amino acid sequence of SEQ ID NO:6, a CDR2 having the amino acid sequence of SEQ ID NO:7 and a CDR3 having the amino acid sequence of SEQ ID NO:8, wherein the anti-CD38 antibody is administered at a dosage of from 45 to 1,800 milligrams.

In one aspect, the anti-CD38 antibody as described herein does not cause hemolytic anemia or thrombocytopenia.

In one aspect, administering the anti-CD38 antibody treatment results in less than 60%, less than 50%, less than 40%, less than 30%, less than 25%, less than 20%, less than 15%, less than 10%, less than 5%, less than 4%, less than 3%, less than 2%, or less than 1% incidence of grade 3 or 4 of one or more treatment-related adverse events (TRAEs) or treatment-emergent adverse events (TEAEs) selected from the group consisting of anemia, hemolytic anemia, neutropenia, thrombocytopenia, fatigue, infusion-related reactions (IRRs), leukopenia, and lymphopenia. A TEAE is an adverse event that is observed or diagnosed up to about 30 days after the last dose of a drug regardless of cause. A TEAE may have any underlying cause related to the disease or treatment that is unrelated to the anti-CD38 antibody or it and can be specifically related to the anti-CD38 antibody. Suitably, administering the anti-CD38 antibody may result in less than 30% incidence of grade 3 or 4 of one or more treatment-emergent adverse events (TEAEs) selected from the group consisting of anemia, hemolytic anemia, thrombocytopenia, fatigue, infusion-related reactions (IRRs), leukopenia, and lymphopenia.

In one aspect, administering the anti-CD38 antibody treatment results in less than 60%, less than 50%, less than 40%, less than 30%, less than 25%, less than 20%, less than 15%, less than 10%, less than 5%, less than 4%, less than 3%, less than 2%, or less than 1% incidence of grade 3 or 4 of one or more treatment-related adverse events (TRAEs) selected from the group consisting of anemia, hemolytic anemia, neutropenia, thrombocytopenia, fatigue, infusion-related reactions (IRRs), leukopenia, and lymphopenia. A TRAE is an adverse event in which a treating physician believes there is a possible causal relationship between the drug used in the treatment and the adverse event. A TRAE thus is considered specifically related to the anti-CD38 antibody. Suitably, administering the anti-CD38 antibody may result in less than 30% incidence of grade 3 or 4 of one or more TRAEs selected from the group consisting of anemia, hemolytic anemia, thrombocytopenia, fatigue, infusion-related reactions (IRRs), leukopenia, and lymphopenia.

In one aspect, administering the anti-CD38 antibody treatment results in less than 10%, less than 9%, less than 8%, less than 7%, less than 6%, less than 5%, less than 4%, less than 3%, less than 2%, less than 1%, depletion of RBCs.

In one aspect, administering the anti-CD38 antibody treatment results in less than 10%, less than 9%, less than 8%, less than 7%, less than 6%, less than 5%, less than 4%, less than 3%, less than 2%, less than 1%, depletion of platelets.

In one aspect, the disease is an autoimmune disease or a cancer.

In one aspect, the autoimmune disease is selected from the group consisting of systemic lupus erythematosus (SLE), rheumatoid arthritis (RA), inflammatory bowel disease (IBD), ulcerative colitis (UC), systemic light chain amyloidosis, and graft-v-host disease.

In one aspect, the hematological cancer is selected from the group consisting of multiple myeloma, chronic lymphoblastic leukemia, chronic lymphocytic leukemia, plasma cell leukemia, acute myeloid leukemia, chronic myeloid leukemia, B-cell lymphoma, and Burkitt lymphoma.

In one aspect, the hematological cancer is multiple myeloma.

In another aspect, the autoimmune disease is systemic light chain amyloidosis.

In one aspect, the VH chain region comprises an amino acid sequence having at least 80% sequence identity to SEQ ID NO: 9 and the VL chain region comprises an amino acid sequence having at least 80% sequence identity to SEQ ID NO: 10. Suitably, the VH chain region may comprise an amino acid sequence having at least 85% sequence identity to SEQ ID NO: 9 and the VL chain region comprises an amino acid sequence having at least 85% sequence identity to SEQ ID NO: 10. Suitably, the VH chain region may comprise an amino acid sequence having at least 90% sequence identity to SEQ ID NO: 9 and the VL chain region comprises an amino acid sequence having at least 90% sequence identity to SEQ ID NO: 10. Suitably, the VH chain region may comprise an amino acid sequence having at least 95% sequence identity to SEQ ID NO: 9 and the VL chain region comprises an amino acid sequence having at least 95% sequence identity to SEQ ID NO: 10. Suitably, the VH chain region may comprise an amino acid sequence having at least 97% sequence identity to SEQ ID NO: 9 and the VL chain region comprises an amino acid sequence having at least 97% sequence identity to SEQ ID NO: 10. Suitably, the VH chain region may comprise an amino acid sequence having at least 99% sequence identity to SEQ ID NO: 9 and the VL chain region comprises an amino acid sequence having at least 99% sequence identity to SEQ ID NO: 10.

Suitably, the VH chain may comprise the CDR sequences as defined by SEQ ID NO: 3, SEQ ID NO: 4 and SEQ ID NO: 5 and the remainder of the sequence may have at least 80% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 80% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 85% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 85% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 90% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 90% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 95% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 95% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 97% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 97% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 99% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 99% sequence identity to SEQ ID NO: 10.

In one aspect, the VH chain region has the amino acid sequence of SEQ ID NO: 9 or a variant thereof with up to three amino acid substitutions and the VL chain region has the amino acid sequence of SEQ ID NO:10 or a variant thereof with up to three amino acid substitutions.

In one aspect, the VH chain region has the amino acid sequence of SEQ ID NO:9 and the VL chain region has the amino acid sequence of SEQ ID NO:10.

In one aspect, the VH chain region comprises an amino acid sequence having at least 80% sequence identity to SEQ ID NO:11 and the VL chain region comprises an amino acid sequence having at least 80% sequence identity to SEQ ID NO:12. Suitably, the VH chain may comprise an amino acid sequence having at least 85% sequence identity to SEQ ID NO:11 and the VL chain region comprises an amino acid sequence having at least 85% sequence identity to SEQ ID NO:12. Suitably, the VH chain may comprise an amino acid sequence having at least 90% sequence identity to SEQ ID NO:11 and the VL chain region comprises an amino acid sequence having at least 90% sequence identity to SEQ ID NO:12. Suitably, the VH chain may comprise an amino acid sequence having at least 95% sequence identity to SEQ ID NO:11 and the VL chain region comprises an amino acid sequence having at least 95% sequence identity to SEQ ID NO:12. Suitably, the VH chain may comprise an amino acid sequence having at least 97% sequence identity to SEQ ID NO:11 and the VL chain region comprises an amino acid sequence having at least 97% sequence identity to SEQ ID NO:12. Suitably, the VH chain may comprise an amino acid sequence having at least 99% sequence identity to SEQ ID NO:11 and the VL chain region comprises an amino acid sequence having at least 99% sequence identity to SEQ ID NO:12.

Suitably, the VH chain may comprise the CDR sequences as defined by SEQ ID NO: 3, SEQ ID NO: 4 and SEQ ID NO: 5 and the remainder of the sequence may have at least 80% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 80% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 85% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 85% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 90% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 90% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 95% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 95% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 97% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 97% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 99% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 99% sequence identity to SEQ ID NO: 12.

In one aspect, the anti-CD38 antibody comprises a heavy chain amino acid sequence of SEQ ID NO:11 or a variant thereof with up to three amino acid substitutions and a light chain amino acid sequence of SEQ ID NO:12 or a variant thereof with up to three amino acid substitutions.

In one aspect, the anti-CD38 antibody comprises a heavy chain amino acid sequence of SEQ ID NO:11 and a light chain amino acid sequence of SEQ ID NO:12.

In one aspect, the therapeutically effective amount is a dosage of from 45 to 1,800 milligrams. Suitably, the therapeutically effective amount may be a dosage of from 45 to 1,200 milligrams. Suitably, the therapeutically effective amount may be a dosage of from 45 to 600 milligrams. Suitably, the therapeutically effective amount may be a dosage of from 45 to 135 milligrams. Suitably, the therapeutically effective amount may be a dosage of from 135 to 1,800 milligrams. Suitably, the therapeutically effective amount may be a dosage of from 135 to 1,200 milligrams. Suitably, the therapeutically effective amount may be a dosage of from 135 to 600 milligrams. Suitably, the therapeutically effective amount may be a dosage of from 600 to 1,800 milligrams. Suitably, the therapeutically effective amount may be a dosage of from 600 to 1,200 milligrams. Suitably, the therapeutically effective amount may be a dosage of from 1,200 to 1,800 milligrams.

In one aspect, the human anti-CD38 antibody is administered in the form of a pharmaceutically acceptable composition.

In another aspect, the invention provides a method for treating a hematological cancer in a subject, the method comprising the step of subcutaneously administering to a subject having a hematological cancer a therapeutically effective amount of an isolated human anti-CD38 antibody sufficient to treat the hematological cancer, wherein the anti-CD38 antibody comprises a VH chain region comprising a CDR1 having the amino acid sequence of SEQ ID NO:3, a CDR2 having the amino acid sequence of SEQ ID NO:4, and a CDR3 having the amino acid sequence of SEQ ID NO:5 or variants of those sequences having up to three amino acid changes; and a VL chain region comprising a CDR1 having the amino acid sequence of SEQ ID NO:6, a CDR2 having the amino acid sequence of SEQ ID NO:7 and a CDR3 having the amino acid sequence of SEQ ID NO:8 or variants of those sequences having up to three amino acid changes and wherein the antibody is administered in a dosage of from 45 to 1,800 milligrams.

In another aspect, the invention provides a method for treating a hematological cancer in a subject, the method comprising the step of subcutaneously administering to a subject having a hematological cancer a therapeutically effective amount of an isolated human anti-CD38 antibody sufficient to treat the hematological cancer, wherein the anti-CD38 antibody comprises a VH chain region comprising a CDR1 having the amino acid sequence of SEQ ID NO:3, a CDR2 having the amino acid sequence of SEQ ID NO:4, and a CDR3 having the amino acid sequence of SEQ ID NO:5 or variants of those sequences having up to three amino acid substitutions; and a VL chain region comprising a CDR1 having the amino acid sequence of SEQ ID NO:6, a CDR2 having the amino acid sequence of SEQ ID NO:7 and a CDR3 having the amino acid sequence of SEQ ID NO:8 or variants of those sequences having up to three amino acid substitutions, wherein the anti-CD38 antibody is administered at a dosage of from 45 to 1,800 milligrams.

In another aspect, the invention provides a method for treating a hematological cancer in a subject, the method comprising the step of subcutaneously administering to a subject having a hematological cancer a therapeutically effective amount of an isolated human anti-CD38 antibody sufficient to treat the hematological cancer, wherein the anti-CD38 antibody comprises a VH chain region comprising a CDR1 having the amino acid sequence of SEQ ID NO:3, a CDR2 having the amino acid sequence of SEQ ID NO:4, and a CDR3 having the amino acid sequence of SEQ ID NO:5; and a VL chain region comprising a CDR1 having the amino acid sequence of SEQ ID NO:6, a CDR2 having the amino acid sequence of SEQ ID NO:7 and a CDR3 having the amino acid sequence of SEQ ID NO:8, wherein the anti-CD38 antibody is administered at a dosage of from 45 to 1,800 milligrams.

In one aspect, the anti-CD38 antibody does not cause hemolytic anemia or thrombocytopenia.

In one aspect, administering the anti-CD38 antibody results in less than 60%, less than 50%, less than 40%, less than 30%, less than 25%, less than 20%, less than 15%, less than 10%, less than 5%, less than 4%, less than 3%, less than 3%, or less than 1% incidence of grade 3 or 4 of one or more treatment-related adverse events (TRAEs) or TEAEs selected from the group consisting of anemia, including hemolytic anemia, thrombocytopenia, fatigue, infusion-related reactions (IRRs), leukopenia, and lymphopenia. Suitably, administering the anti-CD38 antibody may result in less than 30% incidence of grade 3 or 4 of one or more treatment-related adverse events or TEAEs selected from the group consisting of anemia, hemolytic anemia, thrombocytopenia, fatigue, infusion-related reactions (IRRs), leukopenia, and lymphopenia.

In one aspect, administering the anti-CD38 antibody results in less than 10%, less than 9%, less than 8%, less than 7%, less than 6%, less than 5%, less than 4%, less than 3%, less than 2%, less than 1%, depletion of RBCs.

In one aspect, administering the anti-CD38 antibody results in less than 10%, less than 9%, less than 8%, less than 7%, less than 6%, less than 5%, less than 4%, less than 3%, less than 2%, less than 1%, depletion of platelets.

In one aspect, the hematological cancer is selected from the group consisting of multiple myeloma, chronic lymphoblastic leukemia, chronic lymphocytic leukemia, plasma cell leukemia, acute myeloid leukemia, chronic myeloid leukemia, B-cell lymphoma, and Burkitt lymphoma.

In one aspect, the hematological cancer is multiple myeloma.

In one aspect, the VH chain region comprises an amino acid sequence having at least 80% sequence identity to SEQ ID NO:9 and the VL chain region comprises an amino acid sequence having at least 80% sequence identity to SEQ ID NO:10. Suitably, the VH chain region may comprise an amino acid sequence having at least 85% sequence identity to SEQ ID NO: 9 and the VL chain region comprises an amino acid sequence having at least 85% sequence identity to SEQ ID NO: 10. Suitably, the VH chain region may comprise an amino acid sequence having at least 90% sequence identity to SEQ ID NO: 9 and the VL chain region comprises an amino acid sequence having at least 90% sequence identity to SEQ ID NO: 10. Suitably, the VH chain region may comprise an amino acid sequence having at least 95% sequence identity to SEQ ID NO: 9 and the VL chain region comprises an amino acid sequence having at least 95% sequence identity to SEQ ID NO: 10. Suitably, the VH chain region may comprise an amino acid sequence having at least 97% sequence identity to SEQ ID NO: 9 and the VL chain region comprises an amino acid sequence having at least 97% sequence identity to SEQ ID NO: 10. Suitably, the VH chain region may comprise an amino acid sequence having at least 99% sequence identity to SEQ ID NO: 9 and the VL chain region comprises an amino acid sequence having at least 99% sequence identity to SEQ ID NO: 10.

Suitably, the VH chain may comprise the CDR sequences as defined by SEQ ID NO: 3, SEQ ID NO: 4 and SEQ ID NO: 5 and the remainder of the sequence may have at least 80% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 80% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 85% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 85% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 90% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 90% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 95% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 95% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 97% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 97% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 99% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 99% sequence identity to SEQ ID NO: 10.

In one aspect, the VH chain region has the amino acid sequence of SEQ ID NO:9 or a variant thereof with up to three amino acid substitutions and the VL chain region has the amino acid sequence of SEQ ID NO:10 or a variant thereof with up to three amino acid substitutions.

In one aspect, the VH chain region has the amino acid sequence of SEQ ID NO:9 and the VL chain region of has the amino acid sequence of SEQ ID NO:10.

In one aspect, the VH chain region comprises an amino acid sequence having at least 80% sequence identity to SEQ ID NO:11 and the VL chain region comprises an amino acid sequence having at least 80% sequence identity to SEQ ID NO:12. Suitably, the VH chain may comprise an amino acid sequence having at least 85% sequence identity to SEQ ID NO:11 and the VL chain region comprises an amino acid sequence having at least 85% sequence identity to SEQ ID NO:12. Suitably, the VH chain may comprise an amino acid sequence having at least 90% sequence identity to SEQ ID NO:11 and the VL chain region comprises an amino acid sequence having at least 90% sequence identity to SEQ ID NO:12. Suitably, the VH chain may comprise an amino acid sequence having at least 95% sequence identity to SEQ ID NO:11 and the VL chain region comprises an amino acid sequence having at least 95% sequence identity to SEQ ID NO:12. Suitably, the VH chain may comprise an amino acid sequence having at least 97% sequence identity to SEQ ID NO:11 and the VL chain region comprises an amino acid sequence having at least 97% sequence identity to SEQ ID NO:12. Suitably, the VH chain may comprise an amino acid sequence having at least 99% sequence identity to SEQ ID NO:11 and the VL chain region comprises an amino acid sequence having at least 99% sequence identity to SEQ ID NO:12.

Suitably, the VH chain may comprise the CDR sequences as defined by SEQ ID NO: 3, SEQ ID NO: 4 and SEQ ID NO: 5 and the remainder of the sequence may have at least 80% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 80% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 85% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 85% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 90% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 90% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 95% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 95% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 97% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 97% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 99% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 99% sequence identity to SEQ ID NO: 12.

In one aspect, the VH chain region has the amino acid sequence of SEQ ID NO:11 or a variant thereof with up to three amino acid substitutions and the VL chain region has the amino acid sequence of SEQ ID NO:12 or a variant thereof with up to three amino acid substitutions.

In one aspect, the anti-CD38 antibody comprises a heavy chain amino acid sequence of SEQ ID NO:11 and a light chain amino acid sequence of SEQ ID NO:12.

In one aspect, the therapeutically effective amount is a dosage of from 45 to 1,800 milligrams. Suitably, the therapeutically effective amount may be a dosage of from 45 to 1,200 milligrams. Suitably, the therapeutically effective amount may be a dosage of from 45 to 600 milligrams. Suitably, the therapeutically effective amount may be a dosage of from 45 to 135 milligrams. Suitably, the therapeutically effective amount may be a dosage of from 135 to 1,800 milligrams. Suitably, the therapeutically effective amount may be a dosage of from 135 to 1,200 milligrams. Suitably, the therapeutically effective amount may be a dosage of from 135 to 600 milligrams. Suitably, the therapeutically effective amount may be a dosage of from 600 to 1,800 milligrams. Suitably, the therapeutically effective amount may be a dosage of from 600 to 1,200 milligrams. Suitably, the therapeutically effective amount may be a dosage of from 1,200 to 1,800 milligrams.

In one aspect, the human anti-CD38 antibody is administered in the form of a pharmaceutically acceptable composition. Suitably, the pharmaceutically acceptable composition may be suitable for subcutaneous administration.

In another aspect, the invention provides a unit dosage form comprising an isolated antibody that comprises a heavy chain variable region amino acid sequence having at least 80% identity to SEQ ID NO:9 and a light chain variable region amino acid sequence having at least 80% sequence identity to SEQ ID NO:10, wherein the isolated antibody binds to CD38, wherein the unit dosage form is formulated for subcutaneous administration of the antibody at a dosage of from 45 to 1,800 milligrams. Suitably, the VH chain region may comprise an amino acid sequence having at least 85% sequence identity to SEQ ID NO: 9 and the VL chain region comprises an amino acid sequence having at least 85% sequence identity to SEQ ID NO: 10. Suitably, the VH chain region may comprise an amino acid sequence having at least 90% sequence identity to SEQ ID NO: 9 and the VL chain region comprises an amino acid sequence having at least 90% sequence identity to SEQ ID NO: 10. Suitably, the VH chain region may comprise an amino acid sequence having at least 95% sequence identity to SEQ ID NO: 9 and the VL chain region comprises an amino acid sequence having at least 95% sequence identity to SEQ ID NO: 10. Suitably, the VH chain region may comprise an amino acid sequence having at least 97% sequence identity to SEQ ID NO: 9 and the VL chain region comprises an amino acid sequence having at least 97% sequence identity to SEQ ID NO: 10. Suitably, the VH chain region may comprise an amino acid sequence having at least 99% sequence identity to SEQ ID NO: 9 and the VL chain region comprises an amino acid sequence having at least 99% sequence identity to SEQ ID NO: 10.

Suitably, the VH chain may comprise the CDR sequences as defined by SEQ ID NO: 3, SEQ ID NO: 4 and SEQ ID NO: 5 and the remainder of the sequence may have at least 85% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 85% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 85% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 85% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 90% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 90% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 95% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 95% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 97% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 97% sequence identity to SEQ ID NO: 10. Suitably, the VH chain may comprise the CDR sequences as defined by SEQ ID NO: 3, SEQ ID NO: 4 and SEQ ID NO: 5 and the remainder of the sequence may have at least 99% sequence identity to SEQ ID NO: 9 and the VL chain may comprise the CDR sequences as defined by SEQ ID NO: 6, SEQ ID NO: 7 and SEQ ID NO: 8 and the remainder of the VL sequence may have at least 99% sequence identity to SEQ ID NO: 10.

Suitably, the invention may provide a unit dosage form comprising an isolated antibody that comprises a heavy chain variable region amino acid sequence of SEQ ID NO:9 or a variant thereof with up to three amino acid substitutions and a light chain variable region amino acid sequence of SEQ ID NO:10 or a variant thereof with up to three amino acid substitutions, wherein the isolated antibody binds to CD38, wherein the unit dosage form is formulated for subcutaneous administration of the antibody at a dosage of from 45 to 1,800 milligrams.

In another aspect, the invention provides a unit dosage form comprising an isolated antibody that comprises a heavy chain variable region amino acid sequence of SEQ ID NO:9 and a light chain variable region amino acid sequence of SEQ ID NO:10, wherein the isolated antibody binds to CD38 and does not bind significantly to human red blood cells, wherein the unit dosage form is formulated for subcutaneous administration of the antibody at a dosage of from 45 to 1,800 milligrams.

In one aspect, the heavy chain comprises an amino acid sequence having at least 80% sequence identity to SEQ ID NO:11 and the light chain comprises an amino acid sequence having at least 80% identity to SEQ ID NO:12. Suitably, the VH chain may comprise an amino acid sequence having at least 85% sequence identity to SEQ ID NO:11 and the VL chain region comprises an amino acid sequence having at least 85% sequence identity to SEQ ID NO:12. Suitably, the VH chain may comprise an amino acid sequence having at least 90% sequence identity to SEQ ID NO:11 and the VL chain region comprises an amino acid sequence having at least 90% sequence identity to SEQ ID NO:12. Suitably, the VH chain may comprise an amino acid sequence having at least 95% sequence identity to SEQ ID NO:11 and the VL chain region comprises an amino acid sequence having at least 95% sequence identity to SEQ ID NO:12. Suitably, the VH chain may comprise an amino acid sequence having at least 97% sequence identity to SEQ ID NO:11 and the VL chain region comprises an amino acid sequence having at least 97% sequence identity to SEQ ID NO:12. Suitably, the VH chain may comprise an amino acid sequence having at least 99% sequence identity to SEQ ID NO:11 and the VL chain region comprises an amino acid sequence having at least 99% sequence identity to SEQ ID NO:12. Suitably, the VH chain may comprise the CDR sequences as defined by SEQ ID NO: 3, SEQ ID NO: 4 and SEQ ID NO: 5 and the remainder of the sequence may have at least 80% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 80% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 85% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 85% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 90% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 90% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 95% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 95% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 97% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 97% sequence identity to SEQ ID NO: 12. Suitably, the VH chain may comprise the CDR sequences as defined by SEQ ID NO: 3, SEQ ID NO: 4 and SEQ ID NO: 5 and the remainder of the sequence may have at least 99% sequence identity to SEQ ID NO: 11 and the VL chain may comprise the CDR sequences as defined by SEQ ID NO: 6, SEQ ID NO: 7 and SEQ ID NO: 8 and the remainder of the VL sequence may have at least 99% sequence identity to SEQ ID NO: 12.

Suitably, the heavy chain may comprise the amino acid sequence of SEQ ID NO:11 or a variant thereof with up to three amino acid substitutions and the light chain may comprise the amino acid sequence of SEQ ID NO:12 with up to three amino acid substitutions.

In one aspect, the heavy chain may comprise the amino acid sequence of SEQ ID NO:11 and the light chain may comprise the amino acid sequence of SEQ ID NO:12.

In one aspect, the unit dosage form is formulated for subcutaneous administration of the antibody in the treatment of a hematological cancer selected from the group consisting of multiple myeloma, chronic lymphoblastic leukemia, chronic lymphocytic leukemia, plasma cell leukemia, acute myeloid leukemia, chronic myeloid leukemia, B-cell lymphoma, and Burkitt lymphoma.

In one aspect, the hematological cancer is multiple myeloma.

In one aspect, the anti-CD38 antibody does not cause hemolytic anemia or thrombocytopenia.

In one aspect, the anti-CD38 antibody results in less than 10%, less than 9%, less than 8%, less than 7%, less than 6%, less than 5%, less than 4%, less than 3%, less than 2%, less than 1%, depletion of RBCs.

In one aspect, the anti-CD38 antibody results in less than 10%, less than 9%, less than 8%, less than 7%, less than 6%, less than 5%, less than 4%, less than 3%, less than 2%, less than 1%, depletion of platelets.

In one aspect, there is provided a human anti-CD38 antibody for use in therapy, wherein the antibody does not cause a significant level of red blood cell depletion and/or platelet depletion after administration. Suitably, the human anti-CD38 antibody may be administered subcutaneously. Suitably, the antibody may be administered in a dosage of from 45 to 1,800 milligrams.

In one aspect, there is provided a human anti-CD38 antibody for use in therapy, wherein the antibody does not cause a significant level of red blood cell depletion and/or platelet depletion after administration and the human anti-CD38 antibody is administered subcutaneously in a dosage of from 45 to 1,800 milligrams. Suitably, the human anti-CD38 antibody which does not cause a significant level of red blood cell depletion and/or platelet depletion after administration may be an anti-CD38 antibody as defined herein.

In one aspect, there is provided a unit dosage form comprising an isolated antibody that does not cause a significant level of red blood cell depletion and/or platelet depletion after administration, wherein the isolated antibody binds to CD38 and does not bind to human red blood cells, and the unit dosage form is formulated for subcutaneous administration of the antibody at a dosage of from 45 to 1,800 milligrams.

In one aspect, there is provided a human anti-CD38 antibody as defined herein for use in therapy, wherein the human anti-CD38 antibody is formulated for subcutaneous administration. Suitably, the human anti-CD38 antibody is administered subcutaneously.

In one aspect, there is provided a human anti-CD38 antibody as defined herein for use in the treatment of a disease in which binding to CD38 is indicated, wherein the human anti-CD38 antibody is formulated for subcutaneous administration. Suitably, the human anti-CD38 antibody is administered subcutaneously.

In many aspects, the dosage of the administered anti-CD38 antibody as described herein is a weekly dosage.

In one aspect, there is provided a human anti-CD38 antibody as defined herein for use in therapy, wherein the human anti-CD38 antibody is formulated for subcutaneous administration. Suitably, the human anti-CD38 antibody is administered subcutaneously.

Suitably, the human anti-CD38 antibody may be administered in a dosage in the range of from 45 to 1,800 milligrams of antibody. Suitably, the human anti-CD38 antibody may be formulated for subcutaneous administration. Suitably, the human anti-CD38 antibody may be formulated for subcutaneous administration and administered in a dosage in the range of from 45 to 1,800 milligrams of antibody.

In one aspect, there is provided a human anti-CD38 antibody as defined herein for use in the treatment of cancer. Suitably, the cancer may be a hematological cancer.

In one aspect, there is provided a human anti-CD38 antibody as defined herein for use in the treatment of a hematological cancer wherein the human anti-CD38 antibody is formulated for subcutaneous administration. Suitably, the human anti-CD38 antibody may be administered subcutaneously.

Suitably, the human anti-CD38 antibody may be administered in a dosage in the range of from 45 to 1,800 milligram of antibody. Suitably, the human anti-CD38 antibody may be formulated for subcutaneous administration. Suitably, the human anti-CD38 antibody may be formulated for subcutaneous administration and administered in a dosage in the range of from 45 to 1,800 milligrams of antibody.

In one aspect, there is provided a human anti-CD38 antibody as defined herein for use in the treatment of a hematological cancer wherein the human anti-CD38 antibody is formulated for subcutaneous administration and the human anti-CD38 antibody is administered in a dosage in the range of from 45 to 1,800 milligrams of antibody. Suitably, the human anti-CD38 antibody may be administered subcutaneously.

Suitably, the hematological cancer may be multiple myeloma, chronic lymphoblastic leukemia, chronic lymphocytic leukemia, plasma cell leukemia, acute myeloid leukemia, chronic myeloid leukemia, B-cell lymphoma, or Burkitt lymphoma. Suitably, the hematological cancer may be multiple myeloma.

In one aspect, there is provided a human anti-CD38 antibody as defined herein for use in the treatment of an autoimmune disease.

In one aspect, there is provided a human anti-CD38 antibody as defined herein for use in the treatment of an autoimmune disease wherein the human anti-CD38 antibody is formulated for subcutaneous administration. Suitably, the human anti-CD38 antibody may be administered subcutaneously.

Suitably, the human anti-CD38 antibody may be administered in a dosage in the range of from 45 to 1,800 milligrams of antibody. Suitably, the human anti-CD38 antibody may be formulated for subcutaneous administration. Suitably, the human anti-CD38 antibody may be formulated for subcutaneous administration and administered in a dosage in the range of from 45 to 1,800 milligrams of antibody.

Suitably, the autoimmune disease may be systemic lupus erythematosus (SLE), rheumatoid arthritis (RA), inflammatory bowel disease (IBD), ulcerative colitis, systemic light chain amyloidosis, or graft-v-host disease.

In one aspect, there is provided a pharmaceutical composition comprising an isolated human anti-CD38 antibody as defined herein.

In one aspect, there is provided a pharmaceutical composition comprising a unit dosage form according to the present invention.

In one aspect, there is provided a pharmaceutical composition according to the present invention for use in therapy.

In one aspect, there is provided a pharmaceutical composition according to the present invention for use in the treatment of a disease in which binding to CD38 is indicated.

In one aspect, there is provided a pharmaceutical composition according to the present invention for use in treating an autoimmune disease. Suitably, the autoimmune disease may be systemic lupus erythematosus (SLE), rheumatoid arthritis (RA), inflammatory bowel disease (IBD), ulcerative colitis (UC), systemic light chain amyloidosis, or graft-v-host disease. Suitably, the autoimmune disease may be systemic lupus erythematosus (SLE). Suitably, the autoimmune disease may be rheumatoid arthritis (RA). Suitably, the autoimmune disease may be inflammatory bowel disease (IBD). Suitably, the autoimmune disease may be ulcerative colitis (UC). Suitably, the autoimmune disease may be graft-v-host disease.

In another aspect, there is provided a pharmaceutical composition according to the present invention for use in treating cancer. Suitably, the cancer may be a hematological cancer. Suitably, the hematological cancer may be multiple myeloma, chronic lymphoblastic leukemia, chronic lymphocytic leukemia, plasma cell leukemia, acute myeloid leukemia, chronic myeloid leukemia, B-cell lymphoma, or Burkitt lymphoma. Suitably, the hematological cancer may be multiple myeloma. Suitably, the hematological cancer may be chronic lymphoblastic leukemia. Suitably, the hematological cancer may be chronic lymphocytic leukemia. Suitably, the hematological cancer may be plasma cell leukemia. Suitably, the hematological cancer may be acute myeloid leukemia. Suitably, the hematological cancer may be chronic myeloid leukemia. Suitably, the hematological cancer may be B-cell lymphoma. Suitably, the hematological cancer may be Burkitt lymphoma.

In one aspect, there is provided use of an isolated human anti-CD38 antibody as defined herein for the manufacture of a medicament for the treatment of a disease.

In another aspect, there is provided use of a unit dosage form according to the present invention for the manufacture of a medicament for the treatment of a disease.

Suitably, the disease may be one for which binding to CD38 is indicated.

Suitably, the disease may be an autoimmune disease, such as systemic lupus erythematosus (SLE), rheumatoid arthritis (RA), inflammatory bowel disease (IBD), ulcerative colitis (UC), systemic light chain amyloidosis, or graft-v-host disease.

Suitably, the disease may be a cancer. Suitably the cancer may be a hematological cancer, such as multiple myeloma, chronic lymphoblastic leukemia, chronic lymphocytic leukemia, plasma cell leukemia, acute myeloid leukemia, chronic myeloid leukemia, B-cell lymphoma, or Burkitt lymphoma.

Suitably, the medicament may be formulated for subcutaneous administration.

Suitably, the medicament may be formulated to provide a dosage of from 45 to 1,800 milligrams of antibody.

Suitably, the medicament may be formulated for subcutaneous administration and in a dosage of from 45 to 1,800 milligrams of antibody.

These and other embodiments, features and potential advantages will become apparent with reference to the following description and drawings.

### BRIEF DESCRIPTION OF THE DRAWINGS

The objects and features of the invention may be better understood by reference to the drawings described below in which,

FIG. 1 shows a Table of antibodies used for flow cytometric analyses in PD studies.

FIG. 2 shows the PK data of SC dose groups. Anti-drug antibodies (ADA) were detected with a validated qualitative electrochemiluminescent (ECL) assay. The incidence increased over time and affected PK when it reached a specific threshold titer of about 1000 (˜log(7)).

FIG. 3 shows cynomolgus monkey (cyno) PK data and models of AB79. Panels A and B show the raw PK data of the 8 monkey studies, panel A, the first 7 days after the first dose and panel B the entire observation period. The doses were color coded and the SC data was omitted (FIG. 2). Panel C depicts the final PK model structure including target mediated drug disposition (TMDD) marked with a blue box. V_{C} designates the volume of the central compartment where the AB79 concentrations are observed (marked with Conc). V_{P} designates the volume of the peripheral compartment. Rₜₒₜₐₗ represents the compartment of the antibody bound and unbound receptor CD38. KS_{YN} and K_{DEG} designate the production and degradation rate constants of the receptor and K_{INT} the internalization rate constant (complex elimination rate constant). K_{SS} is the steady state constant, defined as K_{SS}=(K_{OFF}+K_{INT})/K_{ON}, where K_{OFF} is the dissociation and K_{ON} the binding rate constant. Panels D-F show the overlays of the linear 2-compartment model predictions (median, 95% prediction interval) without a TMDD component and the observed data of the lowest 3 doses (study 8). Please note the different time scales between panels D, E and F.

FIG. 4 shows the effect of AB79 treatment on RBCs two days post dose in study 7 and total lymphocyte count on the first day post dose.

FIG. 5 shows ADA effects in a 13-week toxicology study. The evaluation refers to the final population PK model (FIG. 1, Table 4). Presented are the following goodness-of-fit (GOF) plots stratified by dose and route of administration (Keizer et al. (2013) CPT Pharmacometrics Syst. Pharmacol. 2:e50): (1) Conditional weighted residuals (CWRES) versus time; (2) Observed concentration versus population model prediction; (3) CWRES versus population model prediction; and (4) Observed concentration versus individual model prediction.

FIG. 6 shows GOF plots for the final Population PK model stratified by dose and route of administration (IV—red, SC—blue).

FIG. 7 shows a comparison of CD38 expression on the surface of human and monkey NK, B, and T cells. The flow cytometric measurements were standardized and the signals are reported in molecules of equivalent soluble fluorescence (MOEF). Human and monkey blood lymphocytes bind similar levels of AB79. Direct comparison of CD38 expression levels on monkey NK cells (CD3−, CD159a+), B cells (CD3−, CD20+) and T cells (CD3+) and human NK cells (CD3−, CD16/CD56+), B cells (CD3−, CD19+) and T cells (CD3+) were evaluated by flow cytometry. The median fluorescent intensity (MFI) for an AB79 staining for each cell population was converted into units MOEF using a standard curve generated using Rainbow Beads (Spherotech; Lake Forest, Ill.). Data shown are from 3 individuals of each species and show the MOEF SD for each cell type. There are differences in CD38 expression between blood lymphocytes, with a higher level of AB79 binding (MOEF) on NK cells>B cells>T cells. The pattern of AB79 binding is similar in blood cells from monkeys, but the level of AB79 binding/CD38 expression is lower.

FIG. 8 shows Inter- and intra-individual variability in the T cell, B cell and NK cell count data of the placebo treated animals.

FIG. 9 shows predose NK, B, and T cell counts (cells per μL) stratified by study (upper row) or sex (lower row).

FIG. 10 shows AB79 dependent NK cell, B cell, and T cell depletion. The presented graphs focus on changes that occurred within the first 7 days after treatment with the first dose of AB79. Thereby, it was possible to pool data from single and multi-dose studies with weekly or every other week dosing schedule. Graphs A-C show the individual minimal cell counts (i.e., the maximal PD effect), the individual cell counts 7 days after the first dose, and the average per dose cell depletion profiles and PK-PD model structure of the NK cells, respectively. Graphs E-F show the same information for the B cells and graphs G-I show the same information for the T cells.

FIG. 11 shows simulated human PK and NK cell, B cell and T cell depletion profiles of AB79. Based on the scaled monkey PK and PK-PD models, 5 single IV and SC dose PK and cell depletion profiles were simulated (from 0.0003 to 1 mg/kg). The left plots show the data after IV administration and the right plots show the data after SC administration. The first row of plots displays the PK profiles. The lower limit of quantification (LLOQ) of 0.05 μg/mL is indicated by a horizontal dashed line. The PK of the lowest dose was completely superimposed by noise and only at doses of 0.03 mg/kg did the PK reach levels above LLOQ.

FIG. 12 shows the plan for an AB79 single rising dose study in healthy volunteers (toxicity study). A total of 6 I.V. and 4 S.C. cohorts in 74 subjects were randomized and received a single dose of AB79. Extensive blinded safety, PK and PD data were reviewed after each cohort before dose escalation. Stopping criteria included depletion of target cells to avoid potential immunosuppression of healthy volunteers. Each subject was followed up for 92 days after dosing.

FIG. 13 shows GOF plots for PK-PD models, stratified on route of administration (IV—red; SC—blue). A) NK cells. B) B cells. C) T cells.

FIG. 14 shows that AB79 mediates cell depletion by antibody dependent cellular cytotoxicity (ADCC) and complement dependent cytotoxicity (CDC). Comparison of CD38 receptor number and susceptibility to ADCC and CDC in human B lineage cell lines. Cell lines with increased CD38 expression were more susceptible to ADCC. No ADCC was seen in a human lymphoblast cell line that did not express CD38 (MV-4-11) or with a Chinese hamster ovary cell line transfected with CD157, a molecule closely related to CD38 (data not shown). EC₅₀, 50% effective concentration; nd, not done; SD, standard deviation.

FIG. 15 shows that AB79 mediates depletion of monkey lymphocytes. AB79 dose-dependently depleted blood NK cells>B cells>T cells in female cynomolgus monkeys (n=4/dose group) after a single IV dose of AB79 as quantified with Flow-Count™ fluorospheres (Beckman-Coulter) using flow cytometry. Samples were collected at pretreatment (Week −1), Day 1: predose, postdose at 15, 30 minutes, 1, 4, 8, 24, 48, 96, and 168 hours, on Days 10, 15, 22, 29, 36, 43, 50, and 57. Only 2-weeks of data are shown for clarity. The mean cell number values were calculated at each time point and were used to calculate % of baseline counts.

FIG. 16 shows that human tetanus toxoid (TTd) recall responses are reduced by AB79 treatment. CB17/SCID mice were treated with anti-asialo GM1 to eliminate NK cells then given 25×10⁶ human peripheral blood lymphocytes. After 7-10 days, serum samples were collected for evaluation of human Ig, the level of Ig was the basis for randomization. Mice were given TTd to induce the recall response and treated with the indicated antibodies twice/week for 10 days. 3 days after the last treatment serum was collected and analyzed for anti-TTd antibodies. AB79 dose-dependently suppressed the TTd recall response. AB79 reduced the recall response to a similar extent as Rituxan (Rtx) (Isotype (Iso), Rtx and AB79 all at 10 mg/kg).

FIG. 17 shows that AB79 does not induce cytokine induction. AB79 (soluble) did not increase IL-6 levels in PBMCs collected from 4 different subjects after 24-hour incubation as compared to IgG1 isotype control PHA (positive control) increased cytokine levels in all subjects demonstrating that cells had the capacity to make IL-6. Similar results were seen with PBMCs stimulated for 48 hours and when IL-2, IL-4, IL-10, GM-CSF, IFNγ and TNFα were tested (data not shown).

FIG. 18A shows the set-up of the dry bound, wet bound and soluble experiment of FIG. 18B (modified from Stebbings et al. (2007) J. Immunol. 179: 3325-3331).

FIG. 18B shows that AB79 does not have agonist activity. AB79 was highly concentrated when it was added to the wells in solution and the liquid allowed to evaporate (Dry Bound) vs. AB79 allowed to bind to wells in solution (Wet Bound) or added directly to PBMCs (Soluble). AB79 did not stimulate IL-6 or IL-2, IL-4, IL-8, IL-10, GM-CSF, IFNγ, or TNFα under any of the conditions tested after 24 hours. IL-8 was constitutively produced by PBMCs and was not altered by any treatment (data not shown).

FIG. 19 shows an evaluation of AB79 binding to cynomolgus monkey CD45+ lymphocytes. Binding of AB79 to CD45+ lymphocytes in unlysed cynomolgus monkey whole blood. CD45+ lymphocytes are gated on and then the binding of AB79 (black histogram) or Isotype Control (red histogram) binding was evaluated. AB79 binding was detected on a subset of the lymphocytes as illustrated in the fraction of cells to the right of the red dashed line. Little to no binding of the isotype control to lymphocytes is observed.

FIG. 20 shows the Mean Observed Cmax and Predose trough (ng/ml) levels (Cycle 1 and Cycle 2). FIG. 20A shows Ab79 Cmax (ng/ml) and FIG. 20B shows Ab79 concentration (ng/ml).

FIG. 21 shows subcutaneously administered Ab79 reduced levels of plasmablasts in blood in a dose-dependent manner.

FIG. 22 shows subcutaneously administered Ab79 reduced levels of plasmablasts in bone marrow aspirates in a dose-dependent manner.

FIG. 23 shows subcutaneously administered Ab79 reduced levels of plasma cells in bone marrow aspirates in a dose-dependent manner.

FIG. 24 shows levels of NK cells in peripheral blood of healthy subjects after a single SC administration of AB79. SC, subcutaneous.

FIG. 25 shows levels of plasmablasts, monocytes, B, T, and NK cells in peripheral blood from healthy subjects after a single injection of placebo control, 0.1, 0.3, or 0.6 mg kg⁻¹ of AB79 SC. [symbol] Absolute monocytes (cells/μL), [symbol] NK cells (cells/μL), [symbol] Total T cells (cells/μL), [symbol] B cells (cells/μL), [symbol] plasmablast cells (cells/μL). The centered curves represent the median. NK, natural killer (cell); SC, subcutaneous.

FIG. 26 shows AB79 and daratumumab binding to human RBCs (individual donor median fluorescence). Peripheral blood from four healthy volunteers incubated with biotin-streptavidin-BV421 AB79 (0, 0.1, 10, 100 μg/ml) or biotin-streptavidin-BV421 daratumumab (0, 0.1, 1, 10, 100 μg/ml) for 3 hours at RT on a gentle shaker in the presence or absence of unlabeled AB79 (500 μg/ml) or unlabeled daratumumab (500 μg/ml). Key: [symbol] AB79-biotin-strep-BV421; [symbol] cold AB79 and AB79-biotin-strep-BV421; [symbol] daratumumab-biotin-strep-BV421; [symbol] cold daratumumab and daratumumab-biotin-strep-BV421.

### DETAILED DESCRIPTION OF THE INVENTION

The present invention relates to methods for treating CD-38 related diseases by the subcutaneous administration of anti-CD38 antibodies.

There are approximately ˜36-fold more CD38 molecules expressed on RBCs than on myeloma cells in the vasculature of patients with active disease. Thus, for example, off-target expression of CD38 may need to be saturated before unbound antibody can pass into the bone marrow and saturate CD38 expressed on myeloma cells. This could explain why other anti-CD38 antibodies in the art, such as daratumumab and isatuximab, which strongly bind to RBCs and platelets, require high dose systemic administration to achieve efficacy.

AB79, daratumumab, isatuximab, and MOR202 are IgG1s that primarily kill tumors by antibody-dependent cellular cytotoxicity (ADCC). This mechanism requires effector cells, such as NK cells, to bind antibodies on target cells and form a lytic synapse to secrete cytotoxic agents in a focused manner. The frequency of these effector cells in blood is orders of magnitude lower than that of RBCs and platelets. For example, the ratio of RBCs to NK cells in blood is 20,000:1. Consequently, effector activity for daratumumab, isatuximab and MOR202 is diverted from tumors because the effector cells are primarily bound by those anti-CD-38 antibodies bound to RBCs and platelets, preventing the formation of a lytic synapse with tumors, which results in a low efficiency of ADCC.

Treatment of patients with anti-CD38 antibodies that bind to RBCs and platelets may result in life threatening side effects. For example, in one study, treatment of relapsed or refractory multiple myeloma with MOR202 resulted in several serious treatment-related adverse events or TEAEs (see, e.g., Raab et al. (2015) Blood 126: 3035). The most common TEAEs at any grade were anemia (15 patients, 34%), fatigue (14 patients, 32%), infusion-related reactions (IRRs) and leukopenia (13 patients, 30% each), lymphopenia and nausea (11 patients, 25% each). Grade >3 TEAEs were reported for 28 patients (64%); the most common included lymphopenia (8 patients, 18%), leukopenia (5 patients, 11%) and hypertension (4 patients, 9%). IRRs arose mainly during the first infusion; all were grade 1-2 except for one patient (grade 3). Infections were commonly reported (26 patients, 59%) but in the majority of the cases were not considered to be treatment-related. MOR202 has only been used clinically via IV infusion.

Other Morphosys antibodies targeting CD38 are known (see, e.g., WO 2006/125640, which discloses four human antibodies: MOR03077, MOR03079, MOR03080, and MOR03100 and two murine antibodies: OKT10 and IB4). These prior art antibodies are inferior to antibodies for use according to the present invention (e.g. AB79) for a variety of reasons. MOR03080 binds to human CD38 and cynomolgus CD38 but with a low affinity to human CD38 (Biacore K_{D}=27.5 nm). OKT10 binds to human CD38 and cynomolgus CD38 but with a low/moderate affinity to human CD38 (Biacore K_{D}=8.28 nm). MOR03079 binds to human CD38 with a high affinity (Biacore K_{D}=2.4 nm) but does not bind to cynomolgus CD38. MOR03100 and MOR03077 bind to human CD38 with moderate or low affinity (Biacore K_{D}=10 nm and 56 nm, respectively). By comparison, antibodies for use according to the present invention (e.g. AB79) binds to human and cynomolgus CD38 with a high affinity to human CD38 (Biacore K_{D}=5.4 nm). Moreover, the prior art antibodies have poor ADCC as well as CDC activity.

An advantage of more efficient ADCC is the ability to deliver an anti-CD38 therapeutic as a low volume injection. If an antibody for use according to the present invention (e.g. AB79) is formulated at a concentration of 100 mg/mL, an efficacious dose for an 80 kg myeloma patient could be administered as a single s.c. injection of <1.0 mL. In contrast, an effective dose of daratumumab or isatuximab delivered into this patient with a comparable form (i.e., 100 mg/mL) would require administering 12.8 mL or 8-16 mL, respectively.

The anti-CD38 methods and unit dosages provide herein subcutaneous administration of therapeutically effective doses of anti-CD38 antibodies, thereby providing unexpected benefits and preventing the side effects, inconvenience, and expense of administering high dose, systemic anti-CD38 antibody therapies.

The present invention provides methods and unit dosage forms for subcutaneous administration of a therapeutically effective amount of an isolated anti-CD38 antibody to a patient in need thereof to treat diseases in which binding to CD38 is indicated, including hematological cancers. In some embodiments, the antibody for subcutaneous administration comprises a heavy chain variable region comprising SEQ ID NO:9 (or a sequence with at least 80%, 85%, 90%, 95%, 97% or 99% sequence identity thereto) and a light chain variable region comprising SEQ ID NO:10 (or a sequence with at least 80%, 85%, 90%, 95%, 97% or 99% sequence identity thereto). The anti-CD38 antibody provided herein is capable of being therapeutically effective when administered by subcutaneous administration.

Another advantage of the anti-CD38 antibodies of the invention is that, unlike some other anti-CD38 antibodies in the clinic, the anti-CD38 antibodies of the present invention (e.g., AB79) are able to bind to cynomolgus monkey (cyno) CD38, providing a useful animal model for preclinical evaluation of dosing, toxicity, efficacy, etc.

Another advantage of the anti-CD38 antibodies of the invention is that they can be used to screen for other antibodies that compete for binding to CD-38 at the same epitope and can be useful in the methods and unit dosages of the invention.

Unless otherwise defined herein, scientific and technical terms used in connection with the present invention shall have the meanings that are commonly understood by those of ordinary skill in the art. The meaning and scope of the terms should be clear. However, in the event of any latent ambiguity, definitions provided herein take precedence over any dictionary or extrinsic definition. Further, unless otherwise required by context, singular terms shall include pluralities and plural terms shall include the singular. The term “or” includes “and/or” unless stated otherwise. Furthermore, the use of the term “including,” “includes,” or “included” is not limiting. Terms such as “element” and “component” encompass both elements and components comprising one unit and elements and components that comprise more than one subunit unless specifically stated otherwise.

Generally, nomenclature used in connection with, and techniques of, cell and tissue culture, molecular biology, immunology, microbiology, genetics and protein and nucleic acid chemistry and hybridization described herein are well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are well-known and commonly used in the art. Standard techniques are used for chemical syntheses, chemical analyses, pharmaceutical preparation, formulation, delivery, and treatment of patients.

All headings and section designations are used for clarity and reference purposes only and are not to be considered limiting in any way. For example, those of skill in the art will appreciate the usefulness of combining various aspects of the disclosure from different headings and sections as appropriate according to the spirit and scope of the invention described herein.

Select terms are defined below in order for the present invention to be more readily understood.

The terms “human CD38” and “human CD38 antigen” refer to the amino acid sequence of SEQ ID NO:1, or a functional fraction thereof, such as an epitope, as defined herein (Table 1). In general, CD38 possesses a short intracytoplasmic tail, a transmembrane domain, and an extracellular domain. The terms “cynomolgus CD38” and “cynomolgus CD38 antigen” refer to the amino acid sequence of SEQ ID NO:2, which is 92% identical to the amino acid sequence of human CD38 (Table 1). Synonyms for CD38 include cyclic ADP ribose hydrolase; cyclic ADP ribose-hydrolase 1; ADP ribosyl cyclase; ADP-ribosyl cyclase 1; cADPr hydrolase 1; CD38-rsl; I-19; NIM-R5 antigen; 2′-phospho-cyclic-ADP-ribose transferase; 2′-phospho-ADP-ribosyl cyclase; 2′-phospho-cyclic-ADP-ribose transferase; 2′-phospho-ADP-ribosyl cyclase; T10.

TABLE 1
Amino Acid Sequence of Human and Cynomolgus Monkey CD38

| | Amino Acid Sequence | |
|---|---|---|
| Species | 12345678901234567890123456789012345678901234567890 | SEQ ID NO |
| Human | MANCEFSPVSGDKPCCRLSRRAQLCLGVSILVLILVVVLAVVVPRWRQQW | 1 |
| CD38 | SGPGTTKRFPETVLARCVKYTEIHPEMRHVDCQSVWDAFKGAFISKHPCN | |
| | ITEEDYQPLMKLGTQTVPCNKILLWSRIKDLAHQFTQVQRDMFTLEDTLL | |
| | GYLADDLTWCGEFNTSKINYQSCPDWRKDCSNNPVSVFWKTVSRRFAEAA | |
| | CDVVHVMLNGSRSKIFDKNSTFGSVEVHNLQPEKVQTLEAWVIHGGREDS | |
| | RDLCQDPTIKELESIISKRNIQFSCKNIYRPDKFLQCVKNPEDSSCTSEI | |
| Cyno | MANCEFSPVSGDKPCCRLSRRAQVCLGVCLLVLLILVVVVAVVLPRWRQQ | 2 |
| CD38 | WSGSGTTSRFPETVLARCVKYTEVHPEMRHVDCQSVWDAFKGAFISKYPC | |
| | NITEEDYQPLVKLGTQTVPCNKTLLWSRIKDLAHQFTQVQRDMFTLEDML | |
| | LGYLADDLTWCGEFNTFEINYQSCPDWRKDCSNNPVSVFWKTVSRRFAET | |
| | ACGVVHVMLNGSRSKIFDKNSTFGSVEVHNLQPEKVQALEAWVIHGGRED | |
| | SRDLCQDPTIKELESIISKRNIRFFCKNIYRPDKFLQCVKNPEDSSCLSG | |
| | I | |
| Human | MAAQGCAASRLLQLLLQLLLLLLLLAAGGARARWRGEGTSAHLRDIFLGR | 13 |
| CD157 | CAEYRALLSPEQRNKNCTAIWEAFKVALDKDPCSVLPSDYDLFINLSRHS | |
| | IPRDKSLFWENSHLLVNSFADNTRRFNIPLSDVLYGRVADFLSWCRQKND | |
| | SGLDYQSCPTSEDCENNPVDSFWKRASIQYSKDSSGVIHVMLNGSEPTGA | |
| | YPIKGFFADYEIPNLQKEKITRIEIWVMHEIGGPNVESCGEGSMKVLEKR | |
| | LKDMGFQYSCINDYRPVKLLQCVDHSTHPDCALKSAAAATQRKAPSLYTE | |
| | QRAGLIIPLFLVLASRTQL | |

The terms “therapeutically effective amount” and “therapeutically effective dosage” refer to an amount of a therapy that is sufficient to reduce or ameliorate the severity and/or duration of a disorder or one or more symptoms thereof, prevent the advancement of a disorder; cause regression of a disorder; prevent the recurrence, development, onset, or progression of one or more symptoms associated with a disorder; or enhance or improve the prophylactic or therapeutic effect(s) of another therapy (e.g., prophylactic or therapeutic agent), at dosages and for periods of time necessary to achieve a desired therapeutic result. A therapeutically effective amount may vary according to factors such as the disease state, age, sex, and weight of the individual, and the ability of the medicaments to elicit a desired response in the individual. A therapeutically effective amount of an antibody is one in which any toxic or detrimental effects of the antibody or antibody portion are outweighed by the therapeutically beneficial effects. A therapeutically effective amount of an antibody for tumor therapy may be measured by its ability to stabilize the progression of disease. The ability of a compound to inhibit cancer may be evaluated in an animal model system predictive of efficacy in human tumors.

The terms “patient” and “subject” include both humans and other animals, particularly mammals. Thus the compositions, dosages, and methods disclosed herein are applicable to both human and veterinary therapies. In one embodiment, the patient is a mammal, for example, a human.

The term “disease in which binding to CD38 is indicated” means a disease in which binding of a binding partner (e.g., an anti-CD38 antibody of the invention) to CD38 provides a prophylactic or curative effect, including the amelioration of one or more symptoms of the disease. Such binding could result in the blocking of other factors or binding partners for CD38, neutralization of CD38, ADCC, CDC, complement activation, or some other mechanism by which the disease is prevented or treated. Factors and binding partners for CD38 include autoantibodies to CD38, which are blocked by the anti-CD38 antibodies of the invention. Such binding may be indicated as a consequence of expression of CD38 by cells or a subset of cells, e.g., MM cells, by which providing a binding partner of CD38 to the subject results in the removal, e.g., lysis, of those cells, e.g., via hemolysis or apoptosis. Such expression of CD38 may be, e.g., normal, overexpressed, inappropriately expressed, or a consequence of activation of CD38, relative to normal cells or relative to other cells types either during a non-disease state or a disease state.

The term “hematologic cancer” refers to malignant neoplasms of blood-forming tissues and encompasses leukemias, lymphomas and multiple myelomas. Non-limiting examples of conditions associated with aberrant CD38 expression include, but are not limited to, multiple myeloma (Jackson et al. (1988) Clin. Exp. Immunol. 72: 351-356); B-cell chronic lymphocytic leukemia (B-CLL) (Dürig et al. (2002) Leukemia 16: 30-35; Morabito et al. (2001) Leukemia Res. 25: 927-932; Marinov et al. (1993) Neoplasma 40(6): 355-358; and Jelinek et al. (2001) Br. J. Haematol. 115: 854-861); acute lymphoblastic leukemia (Keyhani et al. (1999) Leukemia Res. 24: 153-159; and Marinov et al. (1993) Neoplasma 40(6): 355-358); chronic myeloid leukemia (Marinov et al. (1993) Neoplasma 40(6): 355-358); acute myeloid leukemia (Keyhani et al. (1999) Leukemia Res. 24: 153-159); chronic lymphocytic leukemia (CLL); chronic myelogenous leukemia or chronic myeloid leukemia (CML); acute myelogenous leukemia or acute myeloid leukemia (AML); acute lymphocytic leukemia (ALL); hairy cell leukemia (HCL); myelodysplastic syndromes (MDS); and all subtypes and stages (e.g., CML blastic phase (BP), chronic phase (CP), or accelerated phase (AP)) of these leukemias and other hematologic diseases, which are defined by morphological, histochemical and immunological techniques that are well known to those of skill in the art.

The terms “neoplasm” and “neoplastic condition” refer to a condition associated with proliferation of cells characterized by a loss of normal controls that results in one or more symptoms including unregulated growth, lack of differentiation, dedifferentiation, local tissue invasion, and metastasis.

The term “isolated antibody” refers to an antibody that is substantially free of other antibodies having different antigenic specificities. For instance, an isolated antibody that specifically binds to CD38 is substantially free of antibodies that specifically bind antigens other than CD38. An isolated antibody that specifically binds to an epitope, isoform or variant of human CD38 or cynomolgus CD38 may, however, have cross-reactivity to other related antigens, for instance from other species, such as CD38 species homologs. Moreover, an isolated antibody may be substantially free of other cellular material and/or chemicals.

The terms “red blood cells,” “RBCs,” and “erythrocytes” refer to bone marrow derived hemoglobin-containing blood cells that carry oxygen to cells and tissues and carry carbon dioxide back to respiratory organs. RBCs are also referred to as red cells, red blood corpuscles, haematids, and erythroid cells.

The terms “specific binding,” “specifically binds to,” and “is specific for” in reference to the interaction of a particular antibody, protein, or peptide with an antigen, epitope, or other chemical species means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target. The anti-CD38 antibodies of the present invention specifically bind CD38 ligands. The terms “specific binding,” “specifically binds to,” and “is specific for” also mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope. Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

The term “over a period of time” refers to any period of time, e.g., minutes, hours, days, months, or years. For example, over a period of time can refer to at least 10 minutes, at least 15 minutes, at least 30 minutes, at least 60 minutes, at least 75 minutes, at least 90 minutes, at least 105 minutes, at least 120 minutes, at least 3 hours, at least 4 hours, at least 5 hours, at least 6 hours, at least 7 hours, at least 8 hours, at least 9 hours, at least 10 hours, at least 12 hours, at least 14 hours, at least 16, hours, at least 18 hours, at least 20 hours, at least 22 hours, at least one day, at least two days, at least three days, at least 4 days, at least 5 days, at least 6 days, at least a week, at least on month, at least one year, or any interval of time in between. In other words, the antibody from the composition can be absorbed by the individual to whom it is administered over a period of at least 10 minutes, at least 15 minutes, at least 30 minutes, at least 60 minutes, at least 75 minutes, at least 90 minutes, at least 105 minutes, at least 120 minutes, at least 3 hours, at least 4 hours, at least 5 hours, at least 6 hours, at least 7 hours, at least 8 hours, at least 9 hours, at least 10 hours, at least 12 hours, at least 14 hours, at least 16, hours, at least 18 hours, at least 20 hours, at least 22 hours, at least one day, at least two days, at least three days, at least 4 days, at least 5 days, at least 6 days, at least a week, at least on month, at least one year, or any interval of time in between.

A composition that “substantially” comprises a component means that the composition contains more than about 80% by weight of the component. Suitably, the composition may comprise more than about 90% by weight of the component. Suitably, the composition may comprise more than about 95% by weight of the component. Suitably, the composition may comprise more than about 97% by weight of the component. Suitably, the composition may comprise more than about 98% by weight of the component. Suitably the composition may comprise more than about 99% by weight of the component.

The term “about” refers to an extent near in number, degree, volume, time, etc., with only minor variations in dimension of up to 10%.

The term “pharmaceutically acceptable carrier” refers to a pharmaceutically acceptable material, composition or vehicle, suitable for administering compounds of the present invention to mammals. The carriers include liquid or solid filler, diluent, excipient, solvent or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. In one embodiment, the pharmaceutically acceptable carrier is suitable for intravenous administration. In another embodiment, the pharmaceutically acceptable carrier is suitable for locoregional injection. In another embodiment, the pharmaceutically acceptable carrier is suitable for subcutaneous administration. In another embodiment, the pharmaceutically acceptable carrier is suitable for subcutaneous injection.

The term “pharmaceutical composition” refers to preparations suitable for administration to a subject and treatment of disease. When the anti-CD38 antibodies of the present invention are administered as pharmaceuticals to mammals, e.g., humans, they can be administered “as is” or as a pharmaceutical composition containing the anti-CD38 antibody in combination with a pharmaceutically acceptable carrier and/or other excipients. The pharmaceutical composition can be in the form of a unit dosage form for administration of a particular dosage of the anti-CD38 antibody at a particular concentration, a particular amount, or a particular volume. Pharmaceutical compositions comprising the anti-CD38 antibodies, either alone or in combination with prophylactic agents, therapeutic agents, and/or pharmaceutically acceptable carriers are provided. Suitably, the pharmaceutical composition may comprise a unit dosage form according to the present invention either alone or in combination with prophylactic agents, therapeutic agents, and/or pharmaceutically acceptable carriers. Suitably, the pharmaceutical composition may comprise a human anti-CD38 antibody as described herein either alone or in combination with prophylactic agents, therapeutic agents, and/or pharmaceutically acceptable carriers.

Traditional antibody structural units typically comprise a tetramer. Each tetramer is typically composed of two identical pairs of polypeptide chains, each pair having one “light” chain (typically having a molecular weight of about 25 kDa) and one “heavy” chain (typically having a molecular weight of about 50-70 kDa). Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Thus, “isotype” refers to any of the subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE. Therapeutic antibodies can also comprise hybrids of isotypes and/or subclasses.

Each variable heavy (VH) and variable light (VL) region (about 100 to 110 amino acids in length) is composed of three hypervariable regions called “complementarity determining regions” (CDRs) and four framework regions (FRs) (about 15-30 amino acids in length), arranged from amino-terminus to carboxy-terminus in the following order: FR1-CDR1-FR2-CDR2-FR3-CDR3-FR4. “Variable” refers to the fact that the CDRs differ extensively in sequence among antibodies and thereby determines a unique antigen binding site.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region (Kabat et al. (1991) Sequences Of Proteins Of Immunological Interest, 5^{th} Ed. Public Health Service, National Institutes of Health, Bethesda, Md.) and/or those residues forming a hypervariable loop (e.g., residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region (Chothia and Lesk (1987) J. Mol. Biol. 196: 901-917.

The Kabat numbering system is generally used when referring to a residue in the variable domain (approximately, residues 1-107 of the light chain variable region and residues 1-113 of the heavy chain variable region) (e.g., Kabat et al. (1991) Sequences Of Proteins Of Immunological Interest, 5^{th} Ed. Public Health Service, National Institutes of Health, Bethesda, Md.), with the EU number system used for the Fc region.

The term “immunoglobulin (Ig) domain” refers to a region of an immunoglobulin having a distinct tertiary structure. In addition to the variable domains, each heavy and light chain has constant domains: constant heavy (CH) domains; constant light (CL) domains and hinge domains. In the context of IgG antibodies, the IgG isotypes each have three CH regions. The carboxy-terminal portion of each HC and LC defines a constant region primarily responsible for effector function. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Another type of Ig domain of the heavy chain is the hinge region. The term “hinge region” refers to the flexible polypeptide comprising the amino acids between the first and second constant domains of an antibody. Structurally, the IgG CH1 domain ends at EU position 220, and the IgG CH2 domain begins at residue EU position 237. Thus, for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230.

The term “Fc region” refers to the polypeptide comprising the constant region of an antibody excluding the first constant region immunoglobulin domain and in some cases, part of the hinge. Thus, Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

### CD38 Antibodies

Accordingly, the present invention provides isolated anti-CD38 antibodies that specifically bind human and primate CD38 protein that find use in subcutaneous administration methods and unit dosage forms. Of particular use in the present invention are antibodies that bind to both the human and primate CD38 proteins, particularly primates used in clinical testing, such as cynomolgus monkeys (Macacafascicularis, Crab eating macaque, also referred to herein as “cyno”).

In some embodiments, the anti-CD38 antibodies of the invention interact with CD38 at a number of amino acid residues including K121, F135, Q139, D141, M142, E239, W241, S274, C275, K276, F284, V288, K289, N290, P291, E292, D293 and S294 based on human sequence numbering. Suitably, the anti-CD38 antibodies of the invention may interact with CD38 at a number of amino acid residues including K121, F135, Q139, D141, M142, E239, W241, S274, C275, K276, F284, V288, K289, N290, P291, E292, D293 and S294 of SEQ ID NO: 1, based on human sequence numbering. Suitably, the anti-CD38 antibodies of the invention interact with CD38 at a number of amino acid residues including K121, F135, Q139, D141, M142, E239, W241, F274, C275, K276, F284, V288, K289, N290, P291, E292, D293 and S294 of SEQ ID NO: 2. It should be noted that these residues are identical in both human and cynomolgus monkeys, with the exception that S274 is actually F274 in cynomolgus monkeys. These residues may represent the immunodominant epitope and/or residues within the footprint of the specific antigen binding peptide.

In some embodiments, the anti-CD38 antibody for use according to the invention comprises a heavy chain comprising the following CDR amino acid sequences: GFTFDDYG (SEQ ID NO:3; HCDR1 AB79), ISWNGGKT (SEQ ID NO:4; HCDR2 AB79), and ARGSLFHDSSGFYFGH (SEQ ID NO:5; HCDR3 AB79) or variants of those sequences having up to three amino acid changes. In some embodiments, the antibody for use according to the invention comprises a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79) or variants of those sequences having up to three amino acid changes. In some embodiments, the antibody for use according to the invention comprises a heavy chain comprising the following CDR amino acid sequences: GFTFDDYG (SEQ ID NO:3; HCDR1 AB79), ISWNGGKT (SEQ ID NO:4; HCDR2 AB79), ARGSLFHDSSGFYFGH (SEQ ID NO:5; HCDR3 AB79) or variants of those sequences having up to three amino acid changes and a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79) or variants of those sequences having up to three amino acid changes. In some embodiments, the anti-CD38 antibody comprises a heavy chain comprising the following CDR amino acid sequences: GFTFDDYG (SEQ ID NO:3; HCDR1 AB79), ISWNGGKT (SEQ ID NO:4; HCDR2 AB79), and ARGSLFHDSSGFYFGH (SEQ ID NO:5; HCDR3 AB79). In some embodiments, the antibody comprises a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79). In some embodiments, the antibody comprises a heavy chain comprising the following CDR amino acid sequences: GFTFDDYG (SEQ ID NO:3; HCDR1 AB79), ISWNGGKT (SEQ ID NO:4; HCDR2 AB79), ARGSLFHDSSGFYFGH (SEQ ID NO:5; HCDR3 AB79) and a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79). In some embodiments, the antibody comprises a heavy chain comprising an amino acid sequence having at least 80% sequence identity to SEQ ID NO:9. Suitably, the VH chain may comprise the CDR sequences as defined by SEQ ID NO: 3, SEQ ID NO: 4 and SEQ ID NO: 5 and the remainder of the sequence may have at least 80% sequence identity to SEQ ID NO: 9. Suitably, the VH chain may comprise the CDR sequences as defined by SEQ ID NO: 3, SEQ ID NO: 4 and SEQ ID NO: 5 and the remainder of the sequence may have at least 85% sequence identity to SEQ ID NO: 9. Suitably, the VH chain may comprise the CDR sequences as defined by SEQ ID NO: 3, SEQ ID NO: 4 and SEQ ID NO: 5 and the remainder of the sequence may have at least 90% sequence identity to SEQ ID NO: 9. Suitably, the VH chain may comprise the CDR sequences as defined by SEQ ID NO: 3, SEQ ID NO: 4 and SEQ ID NO: 5 and the remainder of the sequence may have at least 95% sequence identity to SEQ ID NO: 9. Suitably, the VH chain may comprise the CDR sequences as defined by SEQ ID NO: 3, SEQ ID NO: 4 and SEQ ID NO: 5 and the remainder of the sequence may have at least 97% sequence identity to SEQ ID NO: 9. Suitably, the VH chain may comprise the CDR sequences as defined by SEQ ID NO: 3, SEQ ID NO: 4 and SEQ ID NO: 5 and the remainder of the sequence may have at least 99% sequence identity to SEQ ID NO: 9.

In some embodiments, the antibody comprises a heavy chain comprising the variable heavy (VH) chain amino acid sequence of SEQ ID NO:9.

(SEQ ID NO: 9)
EVQLLESGGGLVQPGGSLRLSCAASGFTFDDYGMSWVRQAPGKGLEWVSD
ISWNGGKTHYVDSVKGQFTISRDNSKNTLYLQMNSLRAEDTAVYYCARGS
LFHDSSGFYFGHWGQGTLVTVSSASTKGPSVFPLA.

In some embodiments, the antibody comprises a light chain comprising an amino acid sequence having at least 80% sequence identity to SEQ ID NO:10. Suitably, the VL chain may comprise the CDR sequences as defined by SEQ ID NO: 6, SEQ ID NO: 7 and SEQ ID NO: 8 and the remainder of the VL sequence may have at least 80% sequence identity to SEQ ID NO: 10. Suitably, the VL chain may comprise the CDR sequences as defined by SEQ ID NO: 6, SEQ ID NO: 7 and SEQ ID NO: 8 and the remainder of the VL sequence may have at least 85% sequence identity to SEQ ID NO: 10. Suitably, the VL chain may comprise the CDR sequences as defined by SEQ ID NO: 6, SEQ ID NO: 7 and SEQ ID NO: 8 and the remainder of the VL sequence may have at least 90% sequence identity to SEQ ID NO: 10. Suitably, the VL chain may comprise the CDR sequences as defined by SEQ ID NO: 6, SEQ ID NO: 7 and SEQ ID NO: 8 and the remainder of the VL sequence may have at least 95% sequence identity to SEQ ID NO: 10. Suitably, the VL chain may comprise the CDR sequences as defined by SEQ ID NO: 6, SEQ ID NO: 7 and SEQ ID NO: 8 and the remainder of the VL sequence may have at least 97% sequence identity to SEQ ID NO: 10. Suitably, the VL chain may comprise the CDR sequences as defined by SEQ ID NO: 6, SEQ ID NO: 7 and SEQ ID NO: 8 and the remainder of the VL sequence may have at least 99% sequence identity to SEQ ID NO: 10.

In some embodiments, the antibody comprises a light chain comprising the variable light (VL) chain amino acid sequence of SEQ ID NO:10.

(SEQ ID NO: 10)
QSVLTQPPSASGTPGQRVTISCSGSSSNIGDNYVSWYQQLPGTAPKLLIY
RDSQRPSGVPDRFSGSKSGTSASLAISGLRSEDEADYYCQSYDSSLSGSV
FGGGTKLTVLGQPKANPTVTLFPPSSEEL.

In some embodiments, the antibody comprises a heavy chain comprising the VH chain amino acid sequence of SEQ ID NO:9 or a variant thereof as described herein and a light chain comprising the VL chain amino acid sequence of SEQ ID NO:10 or a variant thereof as described herein.

As will be appreciated by those in the art, the variable heavy and light chains can be joined to human IgG constant domain sequences, generally IgG1, IgG2 or IgG4.

In some embodiments, the antibody comprises a heavy chain (HC) comprising an amino acid sequence having at least 80% sequence identity to SEQ ID NO:11. Suitably, the heavy chain may comprise the CDR sequences as defined by SEQ ID NO: 3, SEQ ID NO: 4 and SEQ ID NO: 5 and the remainder of the heavy chain may have at least 80% sequence identity to SEQ ID NO 11. Suitably, the heavy chain may comprise the CDR sequences as defined by SEQ ID NO: 3, SEQ ID NO: 4 and SEQ ID NO: 5 and the remainder of the heavy chain may have at least 85% sequence identity to SEQ ID NO 11. Suitably, the heavy chain may comprise the CDR sequences as defined by SEQ ID NO: 3, SEQ ID NO: 4 and SEQ ID NO: 5 and the remainder of the heavy chain may have at least 90% sequence identity to SEQ ID NO 11. Suitably, the heavy chain may comprise the CDR sequences as defined by SEQ ID NO: 3, SEQ ID NO: 4 and SEQ ID NO: 5 and the remainder of the heavy chain may have at least 95% sequence identity to SEQ ID NO 11. Suitably, the heavy chain may comprise the CDR sequences as defined by SEQ ID NO: 3, SEQ ID NO: 4 and SEQ ID NO: 5 and the remainder of the heavy chain may have at least 97% sequence identity to SEQ ID NO 11. Suitably, the heavy chain may comprise the CDR sequences as defined by SEQ ID NO: 3, SEQ ID NO: 4 and SEQ ID NO: 5 and the remainder of the heavy chain may have at least 99% sequence identity to SEQ ID NO 11.

In some embodiments, the antibody comprises the heavy chain (HC) amino acid sequence of SEQ ID NO:11.

(SEQ ID NO: 11)
EVQLLESGGGLVQPGGSLRLSCAASGFTFDDYGMSWVRQAPGKGLEWVSD
ISWNGGKTHYVDSVKGQFTISRDNSKNTLYLQMNSLRAEDTAVYYCARGS
LFHDSSGFYFGHWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK.

In some embodiments, the antibody comprises a light chain (LC) comprising an amino acid sequence having at least 80% sequence identity to SEQ ID NO:12. Suitably, the light chain may comprise the CDR sequences as defined by SEQ ID NO: 6, SEQ ID NO: 7 and SEQ ID NO: 8 and the remainder of the light chain may have at least 80% sequence identity to SEQ ID NO 12. Suitably, the light chain may comprise the CDR sequences as defined by SEQ ID NO: 6, SEQ ID NO: 7 and SEQ ID NO: 8 and the remainder of the light chain may have at least 85% sequence identity to SEQ ID NO 12. Suitably, the light chain may comprise the CDR sequences as defined by SEQ ID NO: 6, SEQ ID NO: 7 and SEQ ID NO: 8 and the remainder of the light chain may have at least 90% sequence identity to SEQ ID NO 12. Suitably, the light chain may comprise the CDR sequences as defined by SEQ ID NO: 6, SEQ ID NO: 7 and SEQ ID NO: 8 and the remainder of the light chain may have at least 95% sequence identity to SEQ ID NO 12. Suitably, the light chain may comprise the CDR sequences as defined by SEQ ID NO: 6, SEQ ID NO: 7 and SEQ ID NO: 8 and the remainder of the light chain may have at least 97% sequence identity to SEQ ID NO 12. Suitably, the light chain may comprise the CDR sequences as defined by SEQ ID NO: 6, SEQ ID NO: 7 and SEQ ID NO: 8 and the remainder of the light chain may have at least 99% sequence identity to SEQ ID NO 12.

In some embodiments, the antibody comprises the light chain (LC) amino acid sequence of SEQ ID NO:12.

(SEQ ID NO: 12)
QSVLTQPPSASGTPGQRVTISCSGSSSNIGDNYVSWYQQLPGTAPKLLIY
RDSQRPSGVPDRFSGSKSGTSASLAISGLRSEDEADYYCQSYDSSLSGSV
FGGGTKLTVLGQPKANPTVTLFPPSSEELQANKATLVCLISDFYPGAVTV
AWKADGSPVKAGVETTKPSKQSNNKYAASSYLSLTPEQWKSHRSYSCQVT
HEGSTVEKTVAPTECS.

In some embodiments, the antibody comprises the HC amino acid sequence of SEQ ID NO:11 or a variant thereof as described herein and the LC amino acid sequence of SEQ ID NO:12 or a variant thereof as described herein.

The present invention encompasses antibodies that bind to both human and cyno CD38 and interact with at least 80%, 85%, 90%, 91%, 92%, 93%, 94%, 95%, 96%, 97%, 98% or 99% of the following amino acid residues: K121, F135, Q139, D141, M142, E239, W241, S274, C275, K276, F284, V288, K289, N290, P291, E292, D293 and S294 of SEQ ID NO: 1 and SEQ ID NO: 2, based on human numbering. Suitably, the antibody may interact with at least 90% of these amino acid residues. Suitably, the antibody may interact with at least 95% of these amino acid residues. Suitably, the antibody may interact with at least 97% of these amino acid residues. Suitably, the antibody may interact with at least 98% of these amino acid residues. Suitably, the antibody may interact with at least 99% of these amino acid residues. Suitably, the antibody may interact with at least 14 (e.g. at least 15 or at least 16) of the following amino acids: K121, F135, Q139, D141, M142, E239, W241, S274, C275, K276, F284, V288, K289, N290, P291, E292, D293 and S294 of SEQ ID NO: 1 and SEQ ID NO: 2, based on human numbering.

In some embodiments, the antibodies are full length. By “full length antibody” herein is meant the structure that constitutes the natural biological form of an antibody, including variable and constant regions, including one or more modifications as outlined herein.

Alternatively, the antibodies can be a variety of structures, including, but not limited to, antibody fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al. (1989) Nature 341: 544-546) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al. (1988) Science 242: 423-426, Huston et al. (1988) Proc. Natl. Acad. Sci. USA 85: 5879-5883), (viii) bispecific single chain Fv (WO 03/11161) and (ix) “diabodies” or “triabodies”, multivalent or multispecific fragments constructed by gene fusion (Tomlinson et al. (2000) Methods Enzymol. 326: 461-479; WO94/13804; Holliger et al. (1993) Proc. Natl. Acad. Sci. USA 90: 6444-6448).

Suitably, the antibody may be a Fab fragment. Suitably, the antibody may be an Fv fragment. Suitably, the antibody may be an Fd fragment. Suitably, the antibody structure may be isolated CDR regions. Suitably, the antibody may be a F(ab′)2 fragment. Suitably, the antibody may be an scFv fragment.

In some embodiments, the antibodies do not cause a significant level of red blood cell depletion and/or platelet depletion 1 day, 2 days, 4 days, 8 days, 10 days, 15 days, 20 days, 25 days, and/or 30 days after administration.

The term “significant level of cell depletion” may relate to a level of cell depletion which has adverse consequences for the subject.

In some embodiments, the antibodies do not cause a significant level of red blood cell depletion and/or platelet depletion 1 day after administration.

In some embodiments, the antibodies do not cause a significant level of red blood cell depletion and/or platelet depletion 2 days after administration.

In some embodiments, the antibodies do not cause a significant level of red blood cell depletion and/or platelet depletion 4 days after administration.

In some embodiments, the antibodies do not cause a significant level of red blood cell depletion and/or platelet depletion 8 days after administration.

In some embodiments, the antibodies do not cause a significant level of red blood cell depletion and/or platelet depletion 10 days after administration.

In some embodiments, the antibodies do not cause a significant level of red blood cell depletion and/or platelet depletion 15 days after administration.

In some embodiments, the antibodies do not cause a significant level of red blood cell depletion and/or platelet depletion 20 days after administration.

In some embodiments, the antibodies do not cause a significant level of red blood cell depletion and/or platelet depletion 25 days after administration.

In some embodiments, the antibodies do not cause a significant level of red blood cell depletion and/or platelet depletion 30 days after administration.

Suitably, the antibodies for use according to the present invention may result in less than 10%, less than 9%, less than 8%, less than 7%, less than 6%, less than 5%, less than 4%, less than 3%, less than 2%, less than 1% depletion of RBCs after treatment. Suitably, the antibodies for use according to the present invention may result in less than 10%, less than 9%, less than 8%, less than 7%, less than 6%, less than 5%, less than 4%, less than 3%, less than 2%, less than 1% depletion of platelets after treatment.

### Antibody Modifications

The present invention further provides variant anti-CD38 antibodies. That is, there are a number of modifications that can be made to the antibodies of the invention, including, but not limited to, amino acid modifications in the CDRs (affinity maturation), amino acid modifications in the Fc region, glycosylation variants, covalent modifications of other types, etc.

The term “variant” means a polypeptide that differs from that of a parent polypeptide. Amino acid variants can include substitutions, insertions and deletions of amino acids. In general, variants can include any number of modifications, as long as the function of the protein is still present, as described herein. That is, in the case of amino acid variants generated with the CDRs of AB79, for example, the antibody should still specifically bind to both human and cynomolgus CD38. The term “variant Fc region” means an Fc sequence that differs from that of a wild-type or parental Fc sequence by virtue of at least one amino acid modification. Fc variant may refer to the Fc polypeptide itself, compositions comprising the Fc variant polypeptide, or the amino acid sequence. If amino acid variants are generated with the Fc region, for example, the variant antibodies should maintain the required functions for the particular application or indication of the antibody. For example, 1, 2, 3, 4, 5, 6, 7, 8, 9 or 10 amino acid substitutions can be utilized, for example, 1-10, 1-5, 1-4, 1-3, and 1-2 substitutions. Suitable modifications can be made at one or more positions as is generally outlined, for example in U.S. patent application Ser. Nos. 11/841,654; 12/341,769; US Patent Publication Nos. 2004013210; 20050054832; 20060024298; 20060121032; 20060235208; 20070148170; and U.S. Pat. Nos. 6,737,056; 7,670,600; and 6,086,875, all of which are expressly incorporated by reference in their entirety, and in particular for specific amino acid substitutions that increase binding to Fc receptors.

Suitably, the variant maintains the function of the parent sequence, i.e., the variant is a functional variant. Suitably, an antibody comprising a variant sequence maintains the function of the parent antibody, i.e., the antibody comprising a variant sequence is able to bind human CD38. Suitably, treatment with the variant may result in less than 10%, less than 9%, less than 8%, less than 7%, less than 6%, less than 5%, less than 4%, less than 3%, less than 2%, less than 1% depletion of RBCs. Suitably, treatment with the variant may result in less than 10%, less than 9%, less than 8%, less than 7%, less than 6%, less than 5%, less than 4%, less than 3%, less than 2%, less than 1% depletion of platelets.

A variant can be considered in terms of similarity (i.e., amino acid residues having similar chemical properties/functions), preferably a variant is expressed in terms of sequence identity.

Sequence comparisons can be conducted by eye, or more usually, with the aid of readily available sequence comparison programs. These publicly and commercially available computer programs can calculate sequence identity between two or more sequences.

It may be desirable to have from 1-5 modifications in the Fc region of wild-type or engineered proteins, as well as from 1 to 5 modifications in the Fv region, for example. A variant polypeptide sequence will preferably possess at least about 80%, 85%, 90%, 91%, 92%, 93%, 94%, 95%, 96%, 97%, 98% or 99% identity to the parent sequences (e.g., the variable regions, the constant regions, and/or the heavy and light chain sequences for AB79). Suitably, the variant may have at least 80% sequence identity to the parent sequence. Suitably, the variant may have at least 85% sequence identity to the parent sequence. Suitably, the variant may have at least 90% sequence identity to the parent sequence. Suitably, the variant may have at least 92% sequence identity to the parent sequence. Suitably, the variant may have at least 95% sequence identity to the parent sequence. Suitably, the variant may have at least 97% sequence identity to the parent sequence. Suitably, the variant may have at least 98% sequence identity to the parent sequence. Suitably, the variant may have at least 99% sequence identity to the parent sequence.

In one embodiment, the sequence identity is determined across the entirety of the sequence. In one embodiment, the sequence identity is determined across the entirety of the candidate sequence being compared to a sequence recited herein.

The term “amino acid substitution” means the replacement of an amino acid at a particular position in a parent polypeptide sequence with another amino acid. For example, the substitution S100A refers to a variant polypeptide in which the serine at position 100 is replaced with alanine. Suitably the amino acid substitution may be a conservative amino acid substitution. Suitably a variant may comprise one or more, e.g., two or three conservative amino acid substitutions. Amino acids with similar biochemical properties may be defined as amino acids which can be substituted via a conservative substitution.

Unless otherwise explicitly stated herein by way of reference to a specific, individual amino acid, amino acids may be substituted using conservative substitutions as recited below. An aliphatic, polar uncharged amino may be a cysteine, serine, threonine, methionine, asparagine or glutamine residue. An aliphatic, polar charged amino acid may be an aspartic acid, glutamic acid, lysine or arginine residue. An aromatic amino acid may be a histidine, phenylalanine, tryptophan or tyrosine residue. Conservative substitutions may be made, for example according to Table 2 below. Amino acids in the same block in the second column and preferably in the same line in the third column may be substituted for each other:

TABLE 2
Conservative Substitutions

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

The term “amino acid insertion” means the addition of an amino acid at a particular position in a parent polypeptide sequence.

The term “amino acid deletion” means the removal of an amino acid at a particular position in a parent polypeptide sequence.

The terms “parent antibody” and “precursor antibody” mean an unmodified antibody that is subsequently modified to generate a variant. In an embodiment, the parent antibody herein is AB79. In an embodiment, the parent antibody herein comprises a VH chain having the amino acid sequence of SEQ ID NO: 9 and the VL chain having the amino acid sequence of SEQ ID NO: 10. In an embodiment, the parent antibody herein comprises a heavy chain amino acid sequence of SEQ ID NO: 11 and a light chain amino acid sequence of SEQ ID NO: 12. Parent antibody may refer to the polypeptide itself, compositions that comprise the parent antibody, or the amino acid sequence that encodes it. Accordingly, the term “parent Fc polypeptide” means an Fc polypeptide that is modified to generate a variant.

The terms “wild type,” “WT,” and “native” mean an amino acid sequence or a nucleotide sequence that is found in nature, including allelic variations. A WT protein, polypeptide, antibody, immunoglobulin, IgG, etc., has an amino acid sequence or a nucleotide sequence that has not been intentionally modified.

In some embodiments, one or more amino acid modifications are made in one or more of the CDRs of the anti-CD38 antibody. In general, only 1, 2, or 3 amino acids are substituted in any single CDR, and generally no more than from 4, 5, 6, 7, 8 9 or 10 changes are made within a set of CDRs. However, it should be appreciated that any combination of no substitutions, 1, 2 or 3 substitutions in any CDR can be independently and optionally combined with any other substitution.

In some cases, amino acid modifications in the CDRs are referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, it may be desirable to decrease the affinity of an antibody to its antigen.

Affinity maturation can be done to increase the binding affinity of the antibody for the antigen by at least about 10% to 50%, 100%, 150% or more, or from 1 to 5 fold as compared to the “parent” antibody. Preferred affinity matured antibodies will have nanomolar or even picomolar affinities for the target antigen. Affinity matured antibodies are produced by known procedures (e.g., Marks et al. (1992) Biotechnol. 10: 779-783; Barbas et al. (1994) Proc. Nat. Acad. Sci. USA 91: 3809-3813; Shier et al. (1995) Gene 169: 147-155; Yelton et al. (1995) J. Immunol. 155: 1994-2004; Jackson et al. (1995) J. Immunol. 154(7): 3310-9; and Hawkins et al. (1992) J. Mol. Biol. 226: 889-896).

Alternatively, amino acid modifications can be made, e.g. in one or more of the CDRs of the antibodies of the invention that are “silent”, e.g., that do not significantly alter the affinity of the antibody for the antigen. These can be made for a number of reasons, including optimizing expression (as can be done for the nucleic acids encoding the antibodies of the invention).

Thus, included within the definition of the CDRs and antibodies of the invention are variant CDRs and antibodies; that is, the antibodies of the invention can include amino acid modifications in one or more of the CDRs set forth in SEQ ID NO:3 to 8. In addition, as outlined below, amino acid modifications can also independently and optionally be made in any region outside the CDRs, including framework and constant regions.

In some embodiments, variant antibodies of AB79 that are specific for human CD38 (SEQ ID NO:1) and cynomolgus CD38 (SEQ ID NO:2) is described. This antibody is composed of six CDRs, wherein each CDR of this antibody can differ from SEQ ID NO:3, SEQ ID NO:4, SEQ ID NO:5, SEQ ID NO:6, SEQ ID NO:7, and/or SEQ ID NO:8 by 0, 1, or 2 amino acid substitutions.

In addition to the modifications outlined above, other modifications can be made. For example, the molecules may be stabilized by the incorporation of disulphide bridges linking the VH and VL domains (Reiter et al. (1996) Nature Biotech. 14: 1239-1245). In addition, there are a variety of covalent modifications of antibodies that can be made as outlined below.

Covalent modifications of antibodies are included within the scope of this invention, and are generally, but not always, done post-translationally. For example, several types of covalent modifications of the antibody are introduced into the molecule by reacting specific amino acid residues of the antibody with an organic derivatizing agent that is capable of reacting with selected side chains or the N- or C-terminal residues.

In some embodiments, the anti-CD38 antibody of the present invention specifically binds to one or more residues or regions in CD38 but also does not cross-react with other proteins with homology to CD38, such as BST-1 (bone marrow stromal cell antigen-1) and/or Mo5, also called CD157.

Typically, a lack of cross-reactivity means less than about 5% relative competitive inhibition between the molecules when assessed by ELISA and/or FACS analysis using sufficient amounts of the molecules under suitable assay conditions.

### Inhibition of CD38 Activity and Side Effect Reduction

The disclosed antibodies may find use in blocking a ligand-receptor interaction or inhibiting receptor component interaction. The anti-CD38 antibodies of the invention may be “blocking” or “neutralizing.” The term “neutralizing antibody” refers to an antibody for which binding to CD38 results in inhibition of the biological activity of CD38, for example its capacity to interact with ligands, enzymatic activity, signaling capacity and, in particular, its ability to cause activated lymphocytes. Inhibition of the biological activity of CD38 can be assessed by one or more of several standard in vitro or in vivo assays known in the art.

The terms “inhibits binding” and “blocks binding” (e.g., when referring to inhibition/blocking of binding of a CD38 antibody to CD38) encompass both partial and complete inhibition/blocking. The inhibition/blocking of binding of a CD38 antibody to CD38 may reduce or alter the normal level or type of cell signaling that occurs when a CD38 antibody binds to CD38 without inhibition or blocking. Inhibition and blocking are also intended to include any measurable decrease in the binding affinity of a CD38 antibody to CD38 when in contact with an anti-CD38 antibody, as compared to the ligand not in contact with an anti-CD38 antibody, for instance a blocking of binding of a CD38 antibody to CD38 by at least about 10%, 20%, 30%, 40%, 50%, 60%, 70%, 80%, 90%, 99%, or 100%. Suitably, a blocking of binding of a CD38 antibody to CD38 may be at least about 70%. Suitably, a blocking of binding of a CD38 antibody to CD38 may be at least about 80%. Suitably, a blocking of binding of a CD38 antibody to CD38 may be at least about 90%.

The disclosed anti-CD38 antibodies may also inhibit cell growth. The term “inhibits growth” refers to any measurable decrease in cell growth when contacted with an anti-CD38 antibody, as compared to the growth of the same cells not in contact with an anti-CD38 antibody, e.g., an inhibition of growth of a cell culture by at least about 10%, 20%, 30%, 40%, 50%, 60%, 70%, 80%, 90%, 99%, or 100%. Suitably, an inhibition of growth may be at least about 70%. Suitably, an inhibition of growth may be at least about 80%. Suitably, an inhibition of growth may be at least about 90%.

In some embodiments, the disclosed anti-CD38 antibodies are able to deplete activated lymphocytes and plasma cells. The term “depletion” in this context means a measurable decrease in serum levels of activated lymphocytes and/or plasma cells in a subject as compared to untreated subjects. In general, depletions of at least about 10%, 20%, 30%, 40%, 50%, 60%, 70%, 80%, 90%, 99%, or 100% are seen. Suitably, the depletion may be at least 50%. Suitably, the depletion may be at least 60%. Suitably, the depletion may be at least 70%. Suitably, the depletion may be at least 80%. Suitably, the depletion may be at least 90%. Suitably depletion may be 100%. As shown below in the Examples, one particular advantage that the antibodies of the present invention exhibit is the recoverability of these cells after dosing; that is, as is known for some treatments (for example with anti-CD20 antibodies for example), cell depletion can last for long periods of time, causing unwanted side effects. As shown herein, the effects on the activated lymphocytes and/or plasma cells are recoverable.

The anti-CD38 antibodies of the present invention allow for reduced side effects compared to prior art anti-CD38 antibodies. In some embodiments, the antibody for use according to the present invention e.g. AB79 does not induce TEAEs. In some embodiments, the antibody for use according to the present invention e.g. AB79 allows for a reduction in the incidence of TEAEs in a patient population as compared to other anti-CD38 antibodies, such as MOR202. TEAEs are typically referred to by grades 1, 2, 3, 4, and 5, grade 1 being the least severe and grade 5 being the most severe TEAE. Based on FDA and other guidelines for Common Terminology Criteria for Adverse Events (CTCAE) standards for oncology drugs (see, e.g., https://evs.nci.nih.gov/ftpl/CTCAE/CTCAE_4.03_2010-06-14_QuickReference_5x7.pdf; as well as https://ctep.cancer.gov/protocoldevelopment/electronic_applications/ctc.htm; and Nilsson and Koke (2001) Drug Inform. J. 35: 1289-1299) the following is how such grades are generally determined. Grade 1 is mild: asymptomatic or mild symptoms; clinical or diagnostic observations only; no intervention indicated. Grade 2 is moderate: minimal, local or noninvasive intervention indicated; limiting age-appropriate instrumental activities of daily living (“ADL”). Grade 3 is severe or medically significant but not immediately life-threatening: hospitalization or prolongation of hospitalization indicated; disabling; limiting self-care ADL. Grade 4 is life-threatening consequence: urgent intervention indicated. Grade 5 is death related to AE.

In some embodiments, the antibody for use according to the present invention e.g. AB79 allows for a reduction in the grade of the TEAEs in a patient population as compared to other anti-CD38 antibodies, such as MOR202. In some embodiments, the antibody for use according to the present invention e.g. AB79 allows for a reduction in the grade of the TEAEs as compared to other anti-CD38 antibodies from grade 5 to grade 4. In some embodiments, the antibody for use according to the present invention e.g. AB79 allows for a reduction in the grade of the TEAEs as compared to other anti-CD38 antibodies from grade 4 to grade 3. In some embodiments, the antibody for use according to the present invention e.g. AB79 allows for a reduction in the grade of the TEAEs as compared to other anti-CD38 antibodies from grade 3 to grade 2. In some embodiments, the antibody for use according to the present invention e.g. AB79 allows for a reduction in the grade of the TEAEs as compared to other anti-CD38 antibodies from grade 2 to grade 1.

In some embodiments, the antibody for use according to the present invention e.g. AB79 allows for a reduction in grade of one or more TEAEs selected from the group consisting of anemia (including hemolytic anemia), thrombocytopenia, fatigue, infusion-related reactions (IRRs), leukopenia, lymphopenia, and nausea. In some embodiments, the antibody for use according to the present invention e.g. AB79 allows for a reduction in the occurrence of one or more TEAEs selected from the group consisting of anemia (including hemolytic anemia), thrombocytopenia, fatigue, infusion-related reactions (IRRs), leukopenia, lymphopenia, and nausea.

In some embodiments, the anti-CD38 antibody results in less than 50%, less than 40%, less than 30%, less than 20%, less than 10%, less than 5%, less than 4%, less than 3%, less than 2%, or less than 1%, depletion of RBCs. In some embodiments, the AB79 antibody results in less than 50%, less than 40%, less than 30%, less than 20%, less than 10%, less than 5%, less than 4%, less than 3%, less than 2%, or less than 1%, depletion of RBCs. In some embodiments, the AB79 antibody results in less than 10% depletion of RBCs.

In some embodiments, the anti-CD38 antibody results in less than 50%, less than 40%, less than 30%, less than 20%, less than 10%, less than 5%, less than 4%, less than 3%, less than 2%, or less than 1%, depletion of platelets. In some embodiments, the AB79 antibody results in less than 50%, less than 40%, less than 30%, less than 20%, less than 10%, less than 5%, less than 4%, less than 3%, less than 2%, or less than 1%, depletion of platelets. In some embodiments, the AB79 antibody results in less than 10% depletion of platelets.

In some embodiments, a diagnostic test is used for determining the presence and/or grade of anemia, including hemolytic anemia. Diagnostic tests for anemia, including hemolytic anemia including measuring the hemoglobin level. Generally, hemoglobin levels are interpreted as follows: (i) very mild/absent anemia: ≥12.0 g/dL, (ii) mild: 10-12 g/dL, (iii) moderate: 8-10 g/dL, (iv) severe: 6-8 g/dL, and (v) very severe: ≤6 g/dL. Other diagnostic tests for anemia, including hemolytic anemia, include measuring the haptoglobin level. Generally, a haptoglobin level≤25 mg/dL is indicative of the presence of anemia, including hemolytic anemia. Other diagnostic tests include the direct antiglobulin test (DAT) (also referred to as the direct Coombs Test), which is used to determine whether RBCs have been coated in vivo with immunoglobulin, complement, or both.

In some embodiments, a diagnostic test is used for determining the presence and/or grade of thrombocytopenia. Generally, the diagnostic test of thrombocytopenia includes measuring the number of platelets per microliter (L) blood. Normally, there are 150×10³-450×10³ platelets per μL blood. Generally, thrombocytopenia is diagnosed when there is <150×10³ platelets per μL blood. Mild thrombocytopenia is generally diagnosed if there is 70-150×10³ per μL blood. Moderate thrombocytopenia is generally diagnosed if there is 20-70×10³ per μL. Severe thrombocytopenia is generally diagnosed if there is <20×10³ per μL blood.

### Disease Indications

The antibodies, methods, and dosage units of the invention find use in a variety of applications, including treatment or amelioration of CD38-related diseases.

CD38 is expressed in immature hematopoietic cells, down regulated in mature cells, and re-expressed at high levels in activated lymphocytes and plasma cells. For example, high CD38 expression is seen in activated B cells, plasma cells, activated CD4+ T cells, activated CD8+ T cells, NK cells, NKT cells, mature dendritic cells (DCs) and activated monocytes. Certain conditions are associated with cells that express CD38 and certain conditions are associated with the overexpression, high-density expression, or upregulated expression of CD38 on the surfaces of cells. Whether a cell population expresses CD38 or not can be determined by methods known in the art, for example, flow cytometric determination of the percentage of cells in a given population that are labeled by an antibody that specifically binds CD38 or immunohistochemical assays, as are generally described below for diagnostic applications. For example, a population of cells in which CD38 expression is detected in about 10-30% of the cells can be regarded as having weak positivity for CD38; and a population of cells in which CD38 expression is detected in greater than about 30% of the cells can be regarded as definite positivity for CD38 (as in Jackson et al. (1988) Clin. Exp. Immunol. 72: 351-356), though other criteria can be used to determine whether a population of cells expresses CD38. Density of expression on the surface of cells can be determined using methods known in the art, such as, for example, flow cytometric measurement of the mean fluorescence intensity of cells that have been fluorescently labeled using antibodies that specifically bind CD38.

The therapeutic anti-CD38 antibodies of the present invention bind to CD38 positive cells, resulting in depletion of these cells through multiple mechanisms of action, including both CDC and ADCC pathways.

It is known in the art that certain conditions are associated with cells that express CD38, and that certain conditions are associated with the overexpression, high-density expression, or upregulated expression of CD38 on the surfaces of cells. Whether a cell population expresses CD38 or not can be determined by methods known in the art, for example flow cytometric determination of the percentage of cells in a given population that are labeled by an antibody that specifically binds CD38 or immunohistochemical assays, as are generally described below for diagnostic applications. For example, a population of cells in which CD38 expression is detected in about 10-30% of the cells can be regarded as having weak positivity for CD38; and a population of cells in which CD38 expression is detected in greater than about 30% of the cells can be regarded as definite positivity for CD38 (Jackson et al. (1988) Clin. Exp. Immunol. 72: 351-356), though other criteria can be used to determine whether a population of cells expresses CD38. Density of expression on the surfaces of cells can be determined using methods known in the art, such as, for example, flow cytometric measurement of the mean fluorescence intensity of cells that have been fluorescently labeled using antibodies that specifically bind CD38.

In one aspect, the invention provides methods of treating a condition associated with proliferation of cells expressing CD38, comprising administering to a patient a pharmaceutically effective amount of a disclosed antibody. In some embodiments, the condition is cancer, and in particular embodiments, the cancer is a hematological cancer. In some embodiments, the condition is multiple myeloma, chronic lymphoblastic leukemia, chronic lymphocytic leukemia, plasma cell leukemia, acute myeloid leukemia, chronic myeloid leukemia, B-cell lymphoma, or Burkitt lymphoma. In some embodiments, the condition is multiple myeloma.

In some embodiments of the invention, the hematologic cancer is a selected from the group of chronic lymphocytic leukemia, chronic myelogenous leukemia, acute myelogenous leukemia, and acute lymphocytic leukemia. In some embodiments of the invention, the hematologic cancer is chronic lymphocytic leukemia. In some embodiments of the invention, the hematologic cancer is chronic myelogenous leukemia. In some embodiments of the invention, the hematologic cancer is acute myelogenous leukemia. In some embodiments of the invention, the hematologic cancer is acute lymphocytic leukemia.

In some embodiments, the condition is multiple myeloma.

CLL is the most common leukemia of adults in the Western world. CLL involves clonal expansion of mature-appearing lymphocytes involving lymph nodes and other lymphoid tissues with progressive infiltration of bone marrow and presence in the peripheral blood. The B-cell form (B-CLL) represents most cases.

### B Cell Form of Chronic Lymphocytic Leukemia (B-CLL)

B-CLL is an incurable disease characterized by a progressive increase of anergic monoclonal B lineage cells that accumulate in the bone marrow and peripheral blood in a protracted fashion over many years. The expression of CD38 is regarded as an independent poor prognostic factor for B-CLL (Hamblin et al. (2002) Blood 99: 1023-9).

B-CLL is characterized by two subtypes, indolent and aggressive. These clinical phenotypes correlate with the presence or absence of somatic mutations in the immunoglobulin heavy-chain variable region (IgVH) gene. As used herein, indolent B-CLL refers to a disorder in a subject having a mutated IgVH gene and/or presenting with one or more clinical phenotypes associated with indolent B-CLL. As used herein, the phrase aggressive B-CLL refers to a disorder in a subject having an unmutated IgVH gene and/or presenting with one or more clinical phenotypes associated with aggressive B-CLL.

Today's standard therapy of B-CLL is palliative and is mainly carried out with the cytostatic agent chlorambucil or fludarabine. When relapses occur, a combination therapy using fludarabine, cyclophosphamide in combination with rituximab (monoclonal antibody against CD20) or alemtuzumab (monoclonal antibody against CD52) is often initiated. In one study, thirty-five patients with relapsed or refractory aggressive B cell NHL underwent high dose chemotherapy (HCT) followed by rituximab 375 mg/m² weekly for 4 doses starting on day 40 and repeated for four more doses starting on day 180. Rituximab infusions were well tolerated with only one grade 3/4 infusion-related toxicity. The unexpected adverse event noted in this trial was delayed neutropenia in more than half the patients (19/35 patients with 46 episodes of grade 3 or 4 neutropenia; Kosmas et al. (2002) Leukemia 16: 2004-2015, which can be found online at https://www.nature.com/articles/2402639). In another study, six patients received alemtuzumab by intravenous infusion every other day three times a week for 12 weeks. The dose was gradually escalated on daily basis (3, 10 and then 30 mg) until the patient tolerated. The major TEAEs were anemia, neutropenia (6/6 patients each) and thrombocytopenia (5/6 patients) in hematologic adverse events (Ishizawa et al. (2017) Jpn. J. Clin. Oncol. 47(1): 54-60). Thus, there is a critical unmet medical need for the treatment of B-CLL with decreased hematological adverse events. In some embodiments, methods for treating B-CLL using the disclosed anti-CD38 antibodies are provided and, as outlined below, this may be done using combination therapies including optionally and independently any of the above drugs.

### Multiple Myeloma (MM)

Multiple myeloma (MM) is a malignant disorder of the B cell lineage characterized by neoplastic proliferation of plasma cells in the bone marrow. Pharmacologic findings in healthy volunteers supported further investigation in MM (Fedyk et al. (2018) Blood 132:3249, incorporated herein by reference in its entirety). Proliferation of myeloma cells causes a variety of effects, including lytic lesions (holes) in the bone, decreased red blood cell number, production of abnormal proteins (with attendant damage to the kidney, nerves, and other organs), reduced immune system function, and elevated blood calcium levels (hypercalcemia). Currently treatment options include chemotherapy, preferably associated when possible with autologous stem cell transplantation (ASCT). These treatment regimens exhibit moderate response rates. However, only marginal changes in overall survival are observed and the median survival is approximately 3 years. Thus, there is a critical unmet medical need for the treatment of multiple myeloma. In some embodiments, methods for treating multiple myeloma using the disclosed antibodies are provided.

### Monoclonal Gammopathy Of Undetermined Significance (MGUS) And Smoldering Multiple Myeloma (SMM)

Monoclonal gammopathy of undetermined significance (MGUS) and smoldering multiple myeloma (SMM) are asymptomatic, pre-malignant disorders characterized by monoclonal plasma cell proliferation in the bone marrow and absence of end-organ damage.

Smoldering multiple myeloma (SMM) is an asymptomatic proliferative disorder of plasma cells with a high risk of progression to symptomatic, or active multiple myeloma (Kyle et al. (2007) N. Engl. J. Med. 356(25): 2582-2590). International consensus criteria defining SMM were adopted in 2003 and require that a patient have a M-protein level of >30 g/L and/or bone marrow clonal plasma cells>10% (Internat. Myeloma Working Group (2003) Br. J. Haematol. 121: 749-757). The patients must have no organ or related tissue impairment, such as bone lesions or symptoms. Recent studies have identified two subsets of SMM: i) patients with evolving disease and ii) patients with non-evolving disease (Internat. Myeloma Working Group (2003) Br. J. Haematol. 121: 749-757).

SMM resembles monoclonal gammopathy of undetermined significance (MGUS) as end-organ damage is absent (Kyle et al. (2007) N. Engl. J. Med. 356(25): 2582-2590). Clinically, however, SMM is far more likely to progress to active multiple myeloma or amyloidosis at 20 years (78% probability for SMM vs. 21% for MGUS) (Kyle et al. (2007) N. Engl. J. Med. 356(25): 2582-2590).

International consensus criteria defining MGUS require that a patient have a M-protein level of <30 g/L, bone marrow plasma cells<10% and the absence of organ or related tissue impairment, including bone lesions or symptoms (Internat. Myeloma Working Group (2003) Br. J. Haematol. 121: 749-757).

### Systemic Light Chain Amyloidosis

Amyloidosis refers to a family of protein misfolding diseases in which different types of proteins aggregate as extracellular insoluble fibrils. These are complex, multisystem diseases. A common type of systemic amyloidosis is systemic light chain (AL) amyloidosis. (Gertz et al. (2004) Am. Soc. Hematol. 2004: 257-82). Like multiple myeloma, AL amyloidosis is a plasma cell neoplasm. AL amyloidosis is a rare, progressive, and lethal disease of older adults caused by a small clonal plasma cell population in the bone marrow that produces excess monoclonal immunoglobulin free light chains. Once in circulation, these pathologic light chains misfold, aggregate, and deposit as fibrillar material in visceral organs. The amyloid fibril deposits are the same free light chain protein secreted by the clonal plasma cell. (Cohen and Comenzo (2010) Am. J. Hematol. 2010: 287-94; Merlini and Bellotti (2003) New England J. Med. 349(6): 583-96; Murray et al. (2010) Blood (ASH Annual Meeting Abstracts) 116 (21): abstr 1909). End organ damage and ultimately death is caused as a result of this amyloid fibril deposition. Therapies that suppress the clonal plasma cells ameliorate AL amyloidosis disease by removing the factory producing the circulating toxic free light chains, which then can improve organ function and survival. No treatment has received regulatory approval for systemic AL amyloidosis. Agents used are those used to treat multiple myeloma. Thus, there is a critical unmet medical need for the treatment of patients with AL amyloidosis and targeting CD38 on plasma cells is a relevant therapeutic strategy.

### Other CD38 Related Conditions

The antibodies, methods, and dosage units of the invention find use in a variety of applications, including treatment or amelioration of CD38-related diseases, such as diseases and conditions associated with inflammation and immune diseases, particularly diseases associated with activated lymphocytes. The anti-CD38 antibodies of the present invention bind to CD38 positive cells, resulting in depletion of these cells, such as activated lymphocytes, through multiple mechanisms of action, including both CDC and ADCC pathways.

Thus, any autoimmune disease that exhibits either increased expression of CD38 or increased numbers of CD38 expressing cells as a component of the disease may be treated using the antibodies of the invention. These include, but are not limited to, allogenic islet graft rejection, alopecia areata, ankylosing spondylitis, antiphospholipid syndrome, autoimmune Addison's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune dysfunction syndrome, chronic inflammatory demyelinating polyneuropathy, Churg-Strauss syndrome, cicatrical pemphigoid, CREST syndrome, cold agglutinin disease, Crohn's disease, dermatomyositis, discoid lupus, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic pulmonary fibrosis, idiopathic thrombocytopenia purpura (ITP), IgA neuropathy, IgM polyneuropathies, immune mediated thrombocytopenia, juvenile arthritis, Kawasaki's disease, lichen plantus, lupus erythematosus, Meniere's disease, mixed connective tissue disease, multiple sclerosis, type 1 diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobinulinemia, primary biliary cirrhosis, psoriasis, psoriatic arthritis, Reynauld's phenomenon, Reiter's syndrome, rheumatoid arthritis, sarcoidosis, scleroderma, Sjorgen's syndrome, solid organ transplant rejection, stiff-man syndrome, systemic lupus erythematosus, systemic light chain amyloidosis, takayasu arteritis, temporal arteritis/giant cell arteritis, thrombotic thrombocytopenia purpura, ulcerative colitis, uveitis, vasculitides such as dermatitis herpetiformis vasculitis, vitiligo, and Wegner's granulomatosis.

Of particular use in some embodiments are the use of the present antibodies for the use in the diagnosis and/or treatment of a number of diseases, including, but not limited to autoimmune diseases, including but not limited to systemic lupus erythematosus (SLE), rheumatoid arthritis (RA), inflammatory bowel disease (IBD), ulcerative colitis, systemic light chain amyloidosis, and graft-v-host disease. In one aspect, the disease is systemic lupus erythematosus (SLE). In one aspect, the disease is rheumatoid arthritis (RA). In one aspect, the disease is inflammatory bowel disease (IBD). In one aspect the disease is ulcerative colitis. In one aspect, the disease is graft-v-host disease. In one aspect, the disease is systemic light chain amyloidosis.

Thus, for example, patients with high plasma cell content can be treated, such as SLE patients who exhibit high plasma cell levels, as well as RA patients shown to be unresponsive to CD20 based therapies.

### Antibody Compositions for In Vivo Administration

Formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition (1980) Osol, A. Ed.), in the form of lyophilized formulations or aqueous solutions.

The formulations herein may also contain more than one active compound as necessary for the particular indication being treated, preferably those with complementary activities that do not adversely affect each other. For example, it may be desirable to provide antibodies with other specificities. Alternatively, or in addition, the composition may comprise a cytotoxic agent, cytokine, growth inhibitory agent and/or small molecule antagonist. Such molecules are suitably present in combination in amounts that are effective for the purpose intended.

### Subcutaneous Administration

The anti-CD38 antibodies described herein, such as AB79, can be administered at sufficiently dosages that are therapeutically effective, thereby allowing for subcutaneous administration. Subcutaneous administration is a minimally invasive mode of administration and is considered the most versatile and therefore desirable mode of administration that can be used for short term and long term therapies. In some embodiments, subcutaneous administration can be performed by injection. In some embodiments, the site of the injection or device can be rotated when multiple injections or devices are needed.

Accordingly, subcutaneous formulations are much easier for a patient to self-administer, especially since the formulation may have to be taken regularly during the patient's entire life (e.g., starting as early as a child's first year of life). Furthermore, the ease and speed of subcutaneous delivery allows increased patient compliance and quicker access to medication when needed. Thus, the subcutaneous formulations of the anti-CD38 antibodies provided herein provide a substantial benefit over the prior art and solve certain unmet needs.

In some embodiments, the antibodies of the invention are administered to a subject in accordance with known methods via a subcutaneous route. In some embodiments, antibodies of the present invention can be administered by subcutaneous injection. In specific embodiments, the subcutaneous formulation is subcutaneously injected into the same site of a patient (e.g., administered to the upper arm, anterior surface of the thigh, lower portion of the abdomen, or upper back) for repeat or continuous injections. In other embodiments, the subcutaneous formulation is subcutaneously injected into a different or rotating site of a patient. Single or multiple administrations of the formulations may be employed.

In some embodiments, the subcutaneous unit dosage forms described herein can be used for the treatment of cancer. In some embodiments, the subcutaneous unit dosage forms described herein can be used for the treatment of a hematological cancer. In some embodiments, the subcutaneous unit dosage forms described herein can be used for the treatment of multiple myeloma.

In some embodiments, the antibodies of the invention have increased bioavailability as compared to prior art antibodies. In some embodiments, the bioavailability of the antibodies of the present invention is increased 10%, 20%, 30%, 40%, 50%, 60%, 70%, 80%, 90%, or 100% or more as compared to a prior art antibody that binds to human RBCs. In some embodiments, the bioavailability of the antibodies of the present invention that is 110%, 120%, 130%, 140%, 150%, 160%, 170%, 180%, 190%, 200%, 250%, or 300% or more as compared to a prior art antibody that binds to human RBCs. Suitably, the bioavailability may be increased 50%. Suitably, the bioavailability may be increased 60%. Suitably, the bioavailability may be increased 70%. Suitably, the bioavailability may be increased 80%. Suitably, the bioavailability may be increased 90%.

In some embodiments, the increase in bioavailability allows for subcutaneous administration.

In some embodiments, the antibodies of the invention lead to depletion of NK cells, B cells and/or T cells. In some embodiments, the antibodies of the invention allow for increased depletion of NK cells as compared to the depletion of B cells or T cells. In some embodiments, the antibodies of the invention allow for increased depletion of NK cells as compared to B cells, as well as increased depletion of NK cells as compared to T cells. In some embodiments, the antibodies of the invention allow for increased depletion of NK cells as compared to B cells, as well as increased depletion of B cells as compared to T cells. In some embodiments, the antibodies of the invention allow for increased depletion of NK cells as compared to B cells and increased depletion of B cells as compared to T cells. Suitably, the antibodies of the invention may allow for increased depletion of CD38⁺ cells as compared to CD38⁻ cells.

In certain embodiments, the bioavailability of the anti-CD38 antibodies described herein after subcutaneous administration is between at least 50% and at least 80% as compared to intravenous administration normalized for the same dose. In certain embodiments, the bioavailability of the anti-CD38 antibodies described herein after subcutaneous administration is between at least 60% and at least 80% as compared to intravenous administration normalized for the same dose. In certain embodiments, the bioavailability of the anti-CD38 antibodies described herein after subcutaneous administration is between at least 50% and 70% as compared to intravenous administration normalized for the same dose. In certain embodiments, the bioavailability of the anti-CD38 antibodies described herein after subcutaneous administration is between at least 55% and 65% as compared to intravenous administration normalized for the same dose. In certain embodiments, the bioavailability of the anti-CD38 antibodies described herein after subcutaneous administration is between at least 55% and 70% as compared to intravenous administration normalized for the same dose.

In certain embodiments, the bioavailability of the anti-CD38 antibodies described herein after subcutaneous administration is at least 40%, at least 45%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, or at least 85% as compared to intravenous administration normalized for the same dose. Suitably the bioavailability may be at least 50% as compared to intravenous administration normalized for the same dose. Suitably the bioavailability may be at least 60% as compared to intravenous administration normalized for the same dose. Suitably the bioavailability may be at least 70% as compared to intravenous administration normalized for the same dose. Suitably the bioavailability may be at least 80% as compared to intravenous administration normalized for the same dose. Suitably the bioavailability may be at least 90% as compared to intravenous administration normalized for the same dose.

In some embodiments, the present disclosure provides a method wherein the bioavailability of the antibodies of the invention after subcutaneous administration is 50%-80% as compared to intravenous administration normalized for the same dose.

In some embodiments, the present disclosure provides a method wherein the bioavailability of the antibodies of the invention after subcutaneous administration is at least 50% as compared to intravenous administration normalized for the same dose.

In some embodiments, the present disclosure provides a method wherein the bioavailability of the antibodies of the invention after subcutaneous administration is at least 55% as compared to intravenous administration normalized for the same dose.

In some embodiments, the present disclosure provides a method wherein the bioavailability of the antibodies of the invention after subcutaneous administration is at least 60% as compared to intravenous administration normalized for the same dose.

In some embodiments, the present disclosure provides a method wherein the bioavailability of the antibodies of the invention after subcutaneous administration is at least 65% as compared to intravenous administration normalized for the same dose.

In some embodiments, the present disclosure provides a method wherein the bioavailability of the antibodies of the invention after subcutaneous administration is at least 70% as compared to intravenous administration normalized for the same dose.

In some embodiments, the present disclosure provides a method wherein the bioavailability of the antibodies of the invention after subcutaneous administration is at least 75% as compared to intravenous administration normalized for the same dose.

In some embodiments, the present disclosure provides a method wherein the bioavailability of the antibodies of the invention after subcutaneous administration is at least 80% as compared to intravenous administration normalized for the same dose.

In some embodiments, the present disclosure provides the unit dosage form comprising the anti-CD38 antibody as described herein, wherein the anti-CD38 antibody results in less than 10% depletion of RBCs.

In some embodiments, the present disclosure provides the unit dosage form comprising the anti-CD38 antibody as described herein, wherein the anti-CD38 antibody results in less than 10% depletion of platelets.

In certain embodiments, the anti-CD38 antibodies described herein are subcutaneously administered in a single bolus injection. In certain embodiments, the anti-CD38 antibodies described herein are subcutaneously administered monthly. In certain embodiments, the anti-CD38 antibodies described herein are subcutaneously administered every two weeks. In certain embodiments, the anti-CD38 antibodies described herein are subcutaneously administered weekly. In certain embodiments, the anti-CD38 antibodies described herein are subcutaneously administered twice a week. In certain embodiments, the anti-CD38 antibodies described herein are subcutaneously administered daily. In certain embodiments, the anti-CD38 antibodies described herein are subcutaneously administered every 12 hours. In certain embodiments, the anti-CD38 antibodies described herein are subcutaneously administered every 8 hours. In certain embodiments, the anti-CD38 antibodies described herein are subcutaneously administered every six hours. In certain embodiments, the anti-CD38 antibodies described herein are subcutaneously administered every four hours. In certain embodiments, the anti-CD38 antibodies described herein are subcutaneously administered every two hours. In certain embodiments, the anti-CD38 antibodies described herein are subcutaneously administered every hour.

In some embodiments, the subcutaneous unit dosage forms are administered at a dosage of about 45 mgs to about 1,800 mgs. In some embodiments, the subcutaneous unit dosage forms comprise an amount sufficient to administer a dosage of about 135 mgs to about 1,800 mgs. In some embodiments, the subcutaneous unit dosage forms comprise an amount sufficient to administer a dosage of about 600 mgs to about 1,800 mgs. In some embodiments, the subcutaneous unit dosage forms comprise an amount sufficient to administer a dosage of about 1,200 mgs to about 1,800 mgs. In some embodiments, the subcutaneous unit dosage forms comprise an amount sufficient to administer a dosage of about 45 mgs to about 1,200 mgs. In some embodiments, the subcutaneous unit dosage forms comprise an amount sufficient to administer a dosage of about 135 mgs to about 1,200 mgs. In some embodiments, the subcutaneous unit dosage forms comprise an amount sufficient to administer a dosage of about 600 mgs to about 1,200 mgs. In some embodiments, the subcutaneous unit dosage forms comprise an amount sufficient to administer a dosage of about 45 mgs to about 135 mgs. In some embodiments, the subcutaneous unit dosage forms comprise an amount sufficient to administer a dosage of about 45 mgs to about 600 mgs. In some embodiments, the subcutaneous unit dosage forms comprise an amount sufficient to administer a dosage of about 135 mgs to about 600 mgs. In some embodiments, the dosage is in mgs per kilogram bodyweight. In some embodiments, the dosage is a daily dosage.

### Unit Dosage Forms

In some embodiments, the therapeutic anti-CD38 antibodies are formulated as part of a unit dosage form. In some embodiments, the anti-CD38 antibody comprises a heavy chain comprising the following CDR amino acid sequences: GFTFDDYG (SEQ ID NO:3; HCDR1 AB79), ISWNGGKT (SEQ ID NO:4; HCDR2 AB79), and ARGSLFHDSSGFYFGH (SEQ ID NO:5; HCDR3 AB79) or variants of those sequences having up to three amino acid changes. In some embodiments, the antibody comprises a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79) or variants of those sequences having up to three amino acid changes. In some embodiments, the antibody comprises a heavy chain comprising the following CDR amino acid sequences: GFTFDDYG (SEQ ID NO:3; HCDR1 AB79), ISWNGGKT (SEQ ID NO:4; HCDR2 AB79), ARGSLFHDSSGFYFGH (SEQ ID NO:5; HCDR3 AB79) or variants of those sequences having up to three amino acid changes and a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79) or variants of those sequences having up to three amino acid changes. In some embodiments, the antibody comprises a heavy chain comprising the following CDR amino acid sequences: GFTFDDYG (SEQ ID NO:3; HCDR1 AB79), ISWNGGKT (SEQ ID NO:4; HCDR2 AB79), and ARGSLFHDSSGFYFGH (SEQ ID NO:5; HCDR3 AB79). In some embodiments, the antibody comprises a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79). In some embodiments, the antibody comprises a heavy chain comprising the following CDR amino acid sequences: GFTFDDYG (SEQ ID NO:3; HCDR1 AB79), ISWNGGKT (SEQ ID NO:4; HCDR2 AB79), ARGSLFHDSSGFYFGH (SEQ ID NO:5; HCDR3 AB79) and a light chain comprising the following CDR amino acid sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79). In some embodiments, the antibody comprises a heavy chain comprising an amino acid sequence having at least 80% sequence identity to SEQ ID NO:9. Suitably, the heavy chain may comprise the following CDR amino acid sequences: GFTFDDYG (SEQ ID NO:3; HCDR1 AB79), ISWNGGKT (SEQ ID NO:4; HCDR2 AB79), and ARGSLFHDSSGFYFGH (SEQ ID NO:5; HCDR3 AB79) and the remainder of the heavy chain may have at least 80% sequence identity to SEQ ID NO 9. In some embodiments, the antibody comprises a heavy chain comprising the variable heavy (VH) chain amino acid sequence of SEQ ID NO:9.

(SEQ ID NO: 9)
EVQLLESGGGLVQPGGSLRLSCAASGFTFDDYGMSWVRQAPGKGLEWVSD
ISWNGGKTHYVDSVKGQFTISRDNSKNTLYLQMNSLRAEDTAVYYCARGS
LFHDSSGFYFGHWGQGTLVTVSSASTKGPSVFPLA.

In some embodiments, the antibody comprises a light chain comprising an amino acid sequence having at least 80% sequence identity to SEQ ID NO:10. Suitably, the light chain may comprise the following CDR sequences: SSNIGDNY (SEQ ID NO:6; LCDR1 AB79), RDS (SEQ ID NO:7; LCDR2 AB79), and QSYDSSLSGS (SEQ ID NO:8; LCDR3 AB79) and the remainder of the light chain may have at least 80% sequence identity to SEQ ID NO: 10. In some embodiments, the antibody comprises a light chain comprising the variable light (VL) chain amino acid sequence of SEQ ID NO:10.

(SEQ ID NO: 10)
QSVLTQPPSASGTPGQRVTISCSGSSSNIGDNYVSWYQQLPGTAPKLLIY
RDSQRPSGVPDRFSGSKSGTSASLAISGLRSEDEADYYCQSYDSSLSGSV
FGGGTKLTVLGQPKANPTVTLFPPSSEEL.

In some embodiments, the antibody comprises a heavy chain comprising the VH chain amino acid sequence of SEQ ID NO:9 or a variant thereof as described herein and a light chain comprising the VL chain amino acid sequence of SEQ ID NO:10 or a variant thereof as described herein.

As will be appreciated by those in the art, the variable heavy and light chains can be joined to human IgG constant domain sequences, generally IgG1, IgG2 or IgG4. In some embodiments, the antibody comprises a heavy chain (HC) having amino acid sequence with at least 80% sequence identity to SEQ ID NO:11. Suitably, the heavy chain may comprise the CDR sequences as defined by SEQ ID NO: 3, SEQ ID NO: 4 and SEQ ID NO: 5 and the remainder of the heavy chain may have at least 80% sequence identity to SEQ ID NO 11. In some embodiments, the antibody comprises the heavy chain (HC) amino acid sequence of SEQ ID NO:11.

(SEQ ID NO: 11)
EVQLLESGGGLVQPGGSLRLSCAASGFTFDDYGMSWVRQAPGKGLEWVSD
ISWNGGKTHYVDSVKGQFTISRDNSKNTLYLQMNSLRAEDTAVYYCARGS
LFHDSSGFYFGHWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK.

In some embodiments, the antibody comprises a light chain (LC) having amino acid sequence with at least 80% sequence identity to SEQ ID NO:12. Suitably, the light chain may comprise the CDR sequences as defined by SEQ ID NO: 6, SEQ ID NO: 7 and SEQ ID NO: 8 and the remainder of the light chain may have at least 80% sequence identity to SEQ ID NO 12. In some embodiments, the antibody comprises the light chain (LC) amino acid sequence of SEQ ID NO:12.

(SEQ ID NO: 12)
QSVLTQPPSASGTPGQRVTISCSGSSSNIGDNYVSWYQQLPGTAPKLLIY
RDSQRPSGVPDRFSGSKSGTSASLAISGLRSEDEADYYCQSYDSSLSGSV
FGGGTKLTVLGQPKANPTVTLFPPSSEELQANKATLVCLISDFYPGAVTV
AWKADGSPVKAGVETTKPSKQSNNKYAASSYLSLTPEQWKSHRSYSCQVT
HEGSTVEKTVAPTECS.

In some embodiments, the antibody comprises the HC amino acid sequence of SEQ ID NO:11 or a variant thereof as described herein and the LC amino acid sequence of SEQ ID NO:12 or a variant thereof as described herein.

In some embodiments, the formulation comprising the anti-CD38 antibody is a unit dosage form. In some embodiments, the unit dosage form comprises an amount sufficient to administer a dosage of about 45 mgs to about 1,800 mgs. In some embodiments, the unit dosage form comprises an amount sufficient to administer a dosage of about 135 mgs to about 1,800 mgs. In some embodiments, the unit dosage form comprises an amount sufficient to administer a dosage of about 600 mgs to about 1,800 mgs. In some embodiments, the unit dosage form comprises an amount sufficient to administer a dosage of about 1,200 mgs to about 1,800 mgs. In some embodiments, the unit dosage form comprises an amount sufficient to administer a dosage of about 45 mgs to about 1,200 mgs. In some embodiments, the unit dosage form comprises an amount sufficient to administer a dosage of about 135 mgs to about 1,200 mgs. In some embodiments, the unit dosage form comprises an amount sufficient to administer a dosage of about 600 mgs to about 1,200 mgs. In some embodiments, the unit dosage form comprises an amount sufficient to administer a dosage of about 45 mgs to about 135 mgs. In some embodiments, the unit dosage form comprises an amount sufficient to administer a dosage of about 45 mgs to about 600 mgs. In some embodiments, the unit dosage form comprises an amount sufficient to administer a dosage of about 135 mgs to about 600 mgs. In some embodiments, the dosage is in mgs per kilogram bodyweight. In some embodiments, the dosage is a daily dosage.

In some embodiments, the anti-CD38 antibody unit dosage forms provided herein may further comprise one or more pharmaceutically acceptable excipients, carriers, and/or diluents. In some embodiments, the anti-CD38 antibody is provided as a pharmaceutical composition which comprises a unit dosage form according to the present invention. Suitably, the pharmaceutical composition may further comprise one or more pharmaceutically acceptable excipients, carriers, and/or diluents.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Compositions may be formulated in dosage unit form for ease of administration and uniformity of dosage. Dosage unit forms as used herein can, in some embodiments, refer to physically discrete units suited as unitary dosages for the subjects to be treated, each unit containing a predetermined quantity of active compound calculated to produce the desired therapeutic effect in association with the required pharmaceutical carrier.

The specification for the dosage unit forms of the present invention are dictated by and are directly dependent on (a) the unique characteristics of the active compound and the particular therapeutic effect to be achieved, and (b) the limitations inherent in the art of compounding such an active compound for the treatment of an individual.

The efficient dosages and the dosage regimens for the anti-CD38 antibodies used in the present invention depend on the type and severity of the disease or condition to be treated and may be determined by persons skilled in the art.

In one embodiment, the anti-CD38 antibody is administered by subcutaneous administration in a weekly dosage of about 45 to about 1,800 mg. Suitably, the weekly dosage may be about 135 to about 1,800 mg. Suitably, the weekly dosage may be about 600 to about 1,800 mg. Suitably, the weekly dosage may be about 1,200 to about 1,800 mg. Suitably, the weekly dosage may be about 45 to about 1,200 mg. Suitably, the weekly dosage may be about 135 to about 1,200 mg. Suitably, the weekly dosage may be about 600 to about 1,200 mg. Suitably, the weekly dosage may be about 45 to about 135 mg. Suitably, the weekly dosage may be about 45 to about 600 mg. Suitably, the weekly dosage may be about 135 to about 600 mg.

Such administration may be repeated, e.g., 1 to 14 times, such as 3 to 5 times. An exemplary, non-limiting range for a therapeutically effective amount of an anti-CD38 antibody used in the present invention is about 45 to about 1,800 mg. Suitably, the dosage may be about 135 to about 1,800 mg. Suitably, the dosage may be about 600 to about 1,800 mg. Suitably, the dosage may be about 1,200 to about 1,800 mg. Suitably, the dosage may be about 45 to about 1,200 mg. Suitably, the dosage may be about 135 to about 1,200 mg. Suitably, the dosage may be about 600 to about 1,200 mg. Suitably, the dosage may be about 45 to about 135 mg. Suitably, the dosage may be about 45 to about 600 mg. Suitably, the dosage may be about 135 to about 600 mg.

As non-limiting examples, treatment according to the present invention may be provided as a daily dosage of an antibody in an amount of about 45 to about 1,800 mg, such as 45, 60, 80, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340, 1360, 1380, 1400, 1420, 1440, 1460, 1480, 1500, 1520, 1540, 1560, 1580, 1600, 1620, 1640, 1660, 1680, 1700, 1720, 1740, 1760, 1780, or 1800 mg per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, on at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 18, 12, 8, 6, 4, 2, or 1 hour(s), or any combination thereof. Suitably, the daily dosage may be about 45 mg. Suitably, the daily dosage may be about 100 mg. Suitably, the daily dosage may be about 135 mg. Suitably, the daily dosage may be about 150 mg. Suitably, the daily dosage may be about 200 mg. Suitably, the daily dosage may be about 300 mg. Suitably, the daily dosage may be about 400 mg. Suitably, the daily dosage may be about 500 mg. Suitably, the daily dosage may be about 600 mg. Suitably, the daily dosage may be about 700 mg. Suitably, the daily dosage may be about 800 mg. Suitably, the daily dosage may be about 900 mg. Suitably, the daily dosage may be about 1000 mg. Suitably, the daily dosage may be about 1100 mg. Suitably, the daily dosage may be about 1200 mg. Suitably, the daily dosage may be about 1300 mg. Suitably, the daily dosage may be about 1400 mg. Suitably, the daily dosage may be about 1500 mg. Suitably, the daily dosage may be about 1600 mg. Suitably, the daily dosage may be about 1700 mg. Suitably, the daily dosage may be about 1800 mg.

In one embodiment the anti-CD38 antibody is administered in a weekly dosage of about 45 to about 1,800 mg. Suitably, the weekly dosage may be about 135 to about 1,800 mg. Suitably, the weekly dosage may be about 600 to about 1,800 mg. Suitably, the weekly dosage may be about 1,200 to about 1,800 mg. Suitably, the weekly dosage may be about 45 to about 1,200 mg. Suitably, the weekly dosage may be about 135 to about 1,200 mg. Suitably, the weekly dosage may be about 600 to about 1,200 mg. Suitably, the weekly dosage may be about 45 to about 135 mg. Suitably, the weekly dosage may be about 45 to about 600 mg. Suitably, the weekly dosage may be about 135 to about 600 mg. Such administration may be repeated, e.g., 1 to 14 times, such as 3 to 5 times. The administration may be performed by continuous infusion over a period of 1 to 24 hours, such as of 1 to 12 hours. Such regimen may be repeated one or more times as necessary, for example, after 6 months or 12 months. The dosage may be determined or adjusted by measuring the amount of compound of the present invention in the blood upon administration, for instance, by taking a biological sample and using anti-idiotypic antibodies that target the antigen binding region of the anti-CD38 antibody.

In one embodiment, the therapeutic antibody is formulated at 100 mg/ml concentration. In some embodiments, 1.75 mL, 2.0 mL, 2.25 mL or 2.5 mL volume is injected in the thigh, abdomen, or arm. In some embodiments, 1.75 mL, 2.0 mL, 2.25 mL or 2.5 mL volume is injected in the thigh or abdomen. In some embodiments, 2.25 mL volume is injected in the thigh or abdomen. In some embodiments, the dose is administered over a 4-, 6-, 8-, or 10-hour period of time. In some embodiments, the dose is administered over an 8-hour period of time. In some embodiments, 2, 4, 6, or 8 doses are administered. In some embodiments, the doses are administered every 2 hours.

In a further embodiment, the anti-CD38 antibody is administered once weekly for 2 to 12 weeks. Suitably, the antibody may be administered once weekly, such as for 3 to 10 weeks. Suitably, the antibody may be administered once weekly, such as for 4 to 8 weeks. Suitably, the antibody may be administered once weekly, such as for 5 to 7 weeks.

In an embodiment, the anti-CD38 antibody is administered subcutaneously at a frequency that changes over time. Suitably, the antibody may be administered, once weekly for 8 weeks, then once every 2 weeks for 16 weeks, and then once every 4 weeks thereafter in a 28-day treatment cycle until unacceptable toxicities are observed or withdrawal of the subject due to other reasons.

In one embodiment, the anti-CD38 antibody is administered by maintenance therapy, such as, e.g., once a week for a period of 6 months or more.

In one embodiment, the anti-CD38 antibody is administered by a regimen including one infusion of an anti-CD38 antibody followed by an infusion of an anti-CD38 antibody conjugated to a radioisotope. The regimen may be repeated, e.g., 7 to 9 days later.

In one embodiment, the present disclosure provides the unit dosage form comprising the anti-CD38 antibody as described herein, wherein the anti-CD38 antibody results in less than 10% depletion of RBCs.

In one embodiment, the present disclosure provides the unit dosage form comprising the anti-CD38 antibody as described herein, wherein the anti-CD38 antibody results in less than 10% depletion of platelets.

In some embodiments, the anti-CD38 antibody for use according to the invention is used in combination with one or more additional therapeutic agents, e.g., a chemotherapeutic agent. Non-limiting examples of DNA damaging chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-xB inhibitors, including inhibitors of IκB kinase; antibodies which bind to proteins overexpressed, inappropriately expressed, or activated in cancers and thereby downregulate cell replication (e.g., trastuzumab, rituximab, cetuximab, and bevacizumab); and other inhibitors of proteins or enzymes known to be upregulated, overexpressed, inappropriately expressed, or activated in cancers, the inhibition of which downregulates cell replication.

In some embodiments, the antibodies of the invention can be used prior to, concurrent with, or after treatment with Velcade® (bortezomib).

### Treatment Modalities

In the methods of the invention, therapy is used to provide a positive therapeutic response with respect to a disease or condition. The term “positive therapeutic response” refers to an improvement in a disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease. For B cell tumors, the subject may experience a decrease in the so-called B symptoms, e.g., night sweats, fever, weight loss, and/or urticaria. For pre-malignant conditions, therapy with an anti-CD38 therapeutic antibody may block and/or prolong the time before development of a related malignant condition, for example, development of multiple myeloma in subjects suffering from monoclonal gammopathy of undetermined significance (MGUS).

An improvement in the disease may be characterized as a complete response. The term “complete response” refers to the absence of clinically detectable disease with normalization of any previously abnormal radiographic studies, bone marrow, and cerebrospinal fluid (CSF) or abnormal monoclonal protein in the case of myeloma.

Such a response may persist for at least 4 to 8 weeks, or at least 6 to 8 weeks, following treatment according to the methods of the invention. Alternatively, an improvement in the disease may be categorized as being a partial response. The term “partial response” may refer to at least about a 50% decrease in all measurable tumor burden (i.e., the number of malignant cells present in the subject, or the measured bulk of tumor masses or the quantity of abnormal monoclonal protein) in the absence of new lesions, which may persist for 4 to 8 weeks, or 6 to 8 weeks.

Treatment according to the present invention includes a “therapeutically effective amount” of the medicaments used.

The terms “therapeutically effective amount” and “therapeutically effective dosage” refer to an amount of a therapy that is sufficient to reduce or ameliorate the severity and/or duration of a disorder or one or more symptoms thereof, prevent the advancement of a disorder; cause regression of a disorder; prevent the recurrence, development, onset, or progression of one or more symptoms associated with a disorder; or enhance or improve the prophylactic or therapeutic effect(s) of another therapy (e.g., prophylactic or therapeutic agent), at dosages and for periods of time necessary to achieve a desired therapeutic result. A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects. A “therapeutically effective amount” of an antibody for tumor therapy may be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

### Anti-CD38 Antibody Kits

In another aspect of the invention, kits are provided for the treatment of a disease or condition associated with hematological cancers. In one embodiment, the kit comprises a dose of an anti-CD38 antibody described herein, such as AB79. In some embodiments, the kits provided herein may contain one or more dose of a liquid or lyophilized formulation as provided herein. When the kits comprise a lyophilized formulation of an anti-CD38 antibody described herein such as AB79, generally the kits will also contain a suitable liquid for reconstitution of the liquid formulation, for example, sterile water or a pharmaceutically acceptable buffer. In some embodiments, the kits may comprise an anti-CD38 antibody formulation described herein prepackaged in a syringe for subcutaneous administration by a health care professional or for home use.

In certain embodiments, the kit will be for a single administration or dose of an anti-CD38 antibody described herein such as AB79. In other embodiments, the kit may contain multiple doses of an anti-CD38 antibody described herein such as AB79 for subcutaneous administration. In one embodiment, the kit may comprise an anti-CD38 antibody formulation described herein prepackaged in a syringe for subcutaneous administration by a health care professional or for home use.

### Articles Of Manufacture

In other embodiments, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the antibody. The label on, or associated with, the container indicates that the composition is used for treating the condition of choice. The article of manufacture may further comprise a second container comprising a pharmaceutically-acceptable buffer, such as phosphate-buffered saline, Ringer's solution or dextrose solution. It may further include other materials desirable from a commercial and user standpoint, including other buffers, diluents, filters, needles, syringes, and package inserts with instructions for use.

### EXAMPLES

### Example 1: Model-Based Characterization of Anti-CD38 Antibody in Cynomolgus Monkey

Anti-CD38 antibody AB79 binds cynomolgus monkey (cyno) CD38, distinguishing it from daratumumab (Darzalex™), a cytolytic CD38 monoclonal antibody recently approved for the treatment of multiple myeloma. This unique function supported the use of cyno for preclinical studies to characterize AB79 pharmacokinetics (PK), pharmacodynamics (PD) and safety. To this end, assays were developed to measure drug concentrations, immunogenicity, and to quantify T, B, and NK lymphocytes in the blood of cyno monkeys. We assessed these parameters in 8 pharmacological and toxicological preclinical studies. Of the tested cell populations CD38 is most highly expressed on NK cells; therefore, we assume that the drug effect on NK cells comes closest to the effect on the considered target cells, the plasmablasts, plasma cells and other activated lymphocytes.

Data was pooled from 8 studies in healthy monkeys using a dose range of 0.03-100 mg/kg and mathematical models that describe the pharmacokinetics and the exposure-effect relationship for each of the cell types was developed. NK cell depletion was identified as the most sensitive pharmacodynamic effect of AB79. This depletion was described with a turnover model (EC50=34.8 μg/mL on depletion rate) and complete depletion was achieved with an IV dose of 0.3 mg/kg. Also observed were intermediate effects on T cell counts using a direct response model (EC50=9.43 μg/mL) and on B cell counts using a 4-transit-compartment model (EC50=19.3 μg/mL on depletion rate). These analyses substantiated the observation that each of the measured lymphocyte subsets was cleared by AB79 at different rates and required different time spans to deplete the blood compartment.

Mathematical models that describe the PK and PD data are useful tools to gain mechanistic and quantitative insights into the relationships between drug exposure and effect (Friberg et al. (2002) J. Clin. Oncol. 20: 4713-4721; Mager et al. (2003) Drug Metab. Dispos. 31: 510-518; Han and Zhou (2011) Ther. Deliv. 2: 359-368). Typical PK features of IgG antibodies including distribution and elimination, physiological and genetic similarities between monkey and human can be leveraged to explain the pharmacology of AB79 (Glassman and Balthasar (2014) Cancer Biol. Med. 11: 20-33; Kamath (2016) Drug Discov. Today Technol. 21-22: 75-83). In addition, those models have been successfully applied to predict PK concentrations and PD effects in healthy human subjects (Han and Zhou (2011) Ther. Deliv. 2:359-368).

### Materials And Methods

A summary of the monkey studies is shown in Table 3 in chronological order. The single dose studies 2, 7, and 8 were primarily conducted to evaluate PK and PD of intravenously (IV) and subcutaneously (SC) administered AB79. The repeated dose studies were performed to evaluate safety, PK and PD including two 4-week studies (studies 1 and 3) and three 13-week studies under GLP conditions (studies 4, 5, and 6). In the 13-week study 5 a dosing error occurred. Animals of the lowest dose group received 0.01 mg/kg instead of the intended 0.1 mg/kg at one occasion (the second dose) and then continued with 0.1 mg/kg. These data were added to the data set with the correct information of the actually administered dosing amounts. Study 6 repeated the low dose of 0.1 mg/kg QW group of study 5. All animal studies were carried out in accordance with the Guide for the Care and Use of Laboratory Animals as adopted and promulgated by the U.S. National Institutes of Health.

TABLE 3
AB79 Monkey Studies In Chronological Order

| | | Number of | | Number of |
|---|---|---|---|---|
| | | animals | | samples |
| Study | | (female, | Doses | per animal |
| No. | Study Description | male) | (mg/kg) | (PK/PD) |
| 1 | Day 1 (1 mg/kg) + Day 28 (2 mg/kg), IV, | 6 (0, 6) | plc, 1, 2 | 19/10 |
| | PK, PD | | | |
| 2 | Single dose, IV, PK, PD | 9 (0, 9) | plc, 0.3, 3 | 14/9 |
| 3 | 4 weeks tox, once weekly, IV, PK, PD | 12 (4, 8) | plc, 1, 30, 100 | 15/8 |
| 4 | 13 weeks tox, q2wk, IV, PK, PD | 40 (20, 20) | plc, 3, 30, 80 | 47/29 |
| 5 | 13 weeks tox, once weekly, IV, PK, PD | 52 (26, 26) | plc, 0.1, 0.3, 1 | 31/9 |
| 6 | 13 weeks tox, once weekly, IV, PK, PD | 20 (20, 0) | plc, 0.1 | 31/10 |
| 7 | Single dose, IV/SC, PK, PD | 12 (12, 0) | 0.1, 0.3, 1 | 16/16 |
| 8 | Single dose, IV/SC, PK, PD | 24 (24, 0) | 0.03, 0.1, 0.3 | 19/19 |

IV: intravenous 30 min infusion (studies 1-4) or bolus (studies 5-8), SC: subcutaneous injection (group 4 of study 7 and 3 groups of study 8), PK: dense PK sampling, PD: dense sampling of whole blood for flow cytometry analyses yielding cell count data of T, B, and NK cells. plc—placebo, “4 weeks” or “13 weeks” describe the duration of the treatment period, tox: toxicology study, q2wk: every other week dosing schedule.

### Bioanalytics

PK was analyzed using a validated method developed and performed by Charles River Laboratories (Reno, Nev.). Briefly, the concentration of AB79 was measured in monkey serum using an indirect enzyme linked immunosorbent assay (ELISA). A 96-well microtiter format was coated with an anti-idiotypic antibody against AB79. Blanks, standards, and quality control (QC) samples containing AB79 at various concentrations were added to the plate, and incubated for 55-65 minutes at room temperature (RT). After washing the microtiter plate, a peroxidase conjugated affinipure mouse anti-human IgG (Peroxidase AffiniPure Mouse Anti-Human IgG, Fcγ Fragment Specific; Jackson ImmunoResearch) was added, and incubated on the plate for an additional 55-65 minutes. The plate was washed again, and tetramethylbenzidine (TMB) was added to the wells to generate a chromophore, and the development of color was stopped by the addition of a stopping solution (2N sulfuric acid). The absorbance at 450 nm was measured using a SPECTRAmax® 190 microplate reader (Molecular Devices) and AB79 concentrations were calculated using a 4-parameter logistic weighted (1/y²) standard calibration curve. In study 1 (Table 3) the lower limit of quantification (LLOQ) of AB79 in serum was 0.061 μg/mL and in all other studies it was 0.05 μg/mL.

### Determination of Anti-AB79 Antibodies (Immunogenicity)

Anti-drug antibodies (ADA) screening of monkey serum was analyzed using a qualitative electrochemiluminescent (ECL) method, validated and performed by Charles River Laboratories (Reno, Nev.). Briefly, undiluted serum samples were incubated with 300 mM acetic acid. Acid-dissociated samples were incubated in a mixture of biotinylated AB79, AB79 labeled with SULFO-TAG (Meso Scale Diagnostics, labeled at Charles River Laboratories) and 1.5 M Trizma base to neutralize the acid and form an immune complex. This complex was then added to a streptavidin-coated MSD plate (Meso Scale Diagnostics) and allowed to bind. After washing, the complex was detected by the addition of MSD read buffer T (Meso Scale Diagnostics) to the plate and subsequent excitation of the SULFO-TAG™ via an electrochemical reaction of Ru(bpy)3 to generate luminescence (light), which was read using the MSD Sector 6000 (Meso Scale Diagnostics). The quantity of luminescence correlated with the level of monkey anti-AB79 antibodies present in the serum of individual samples.

### Characterization of Blood Cells

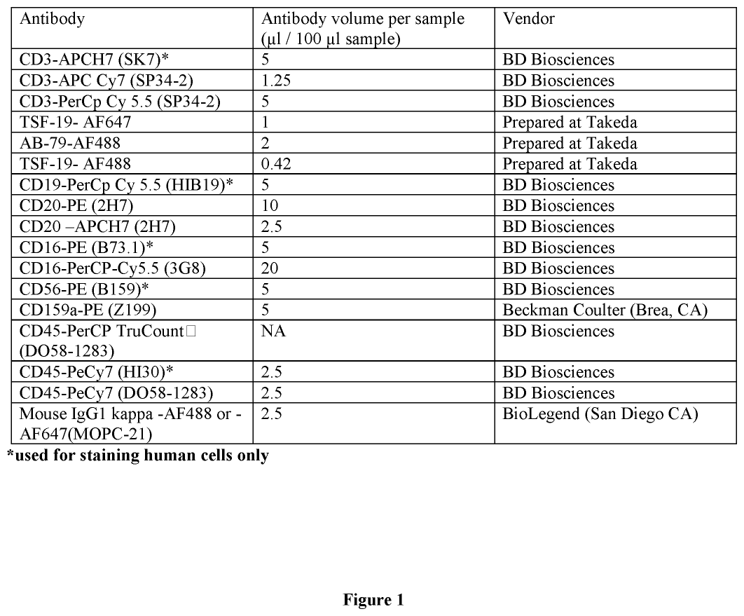

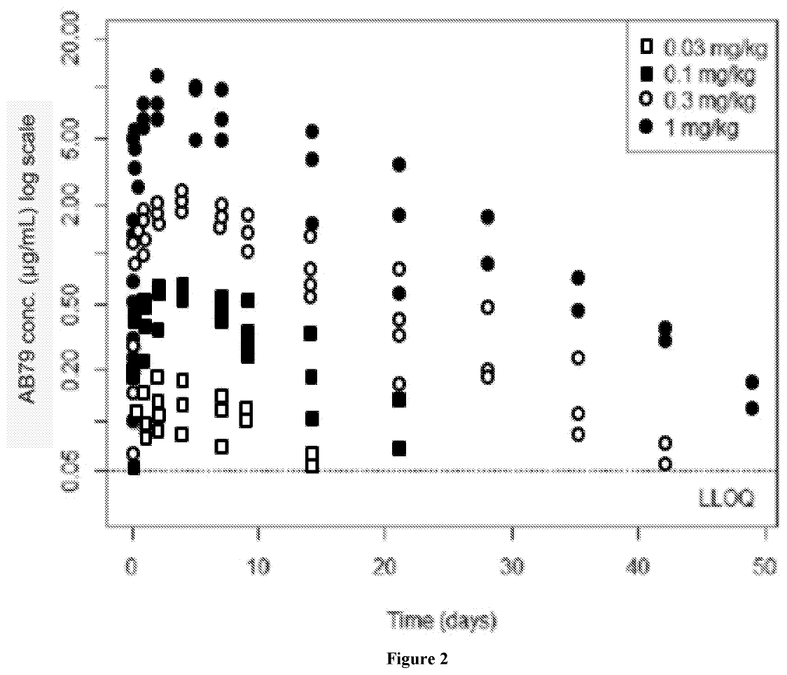

To evaluate and compare the level of AB79 binding between humans and monkeys, blood samples from each were collected into sodium heparin tubes. An aliquot of blood (100 μL) was mixed with appropriate volume of antibody (FIG. 1) and incubated for 15-20 minutes at RT in the dark. After incubation, 1 mL of BD FACS lyse (1×; BD Biosciences; San Jose, Calif.) was added to lyse red blood cells and the cells incubated for 10 minutes at RT in the dark, then centrifuged, decanted and resuspended in 1 mL of staining buffer with bovine serum albumin (BD Biosciences). The cells were centrifuged a second time, decanted and 250 μL of Flow Fix (1% paraformaldehyde in calcium and magnesium free Dulbecco's-PBS (Life Technologies, Carlsbad, Calif.) and fluorescence measured by flow cytometric analyses using a FACSCanto™ II Flow Cytometer (BD Biosciences). Monkey NK cells (CD3−, CD159a+), B cells (CD3−, CD20+) and T cells (CD3+) and human NK cells (CD3−, CD16/CD56+), B cells (CD3−, CD19+) and T cells (CD3+) were measured. The mean fluorescence intensity for AB79 staining for each cell population was converted into units of molecules of equivalent soluble fluorescence (MOEF) using a standard curve generated using Rainbow Beads (Spherotech; Lake Forest, Ill.).

In studies outlined in Table 3, cells were stained and analyzed using a validated method developed and performed by Charles River Laboratories (Reno, Nev.). Monkey blood samples were collected into sodium heparin tubes before and at multiple times after AB79 treatment and specific lymphocyte populations measured by flow cytometric analyses using FACSCanto™ II Flow Cytometer (BD Biosciences). Commercial antibodies and a CD38 antibody (Ab19; U.S. Pat. No. 8,362,211) were titered to optimal concentrations for staining. Monkey CD38+/−, T cell (CD3+), B cell (CD3−/CD20+), and natural killer (NK) cell (CD3−/CD20−/CD16+) populations were identified and lymphocytes quantified using CD45TruCount™ tubes (BD Biosciences). Approximately 100 μL aliquots of each blood sample were placed into an appropriate well of a 96-well plate and antibodies added at the indicated volume, mixed and incubated for a minimum of 30 minutes at RT in the dark. After incubation, red blood cells were lysed, samples mixed and incubated at RT for an additional 10 minutes in the dark. The plate was centrifuged and the supernatant was decanted. The cell pellet was then resuspended in 1,800 μL of stain buffer, samples mixed, centrifuged and the supernatant decanted. The cell pellet was resuspended in 500 μL of stain buffer with fetal bovine serum and approximately 300 μL of the cell suspension transferred to a 96-well v-bottom plate for analysis. The NK cell percentages, as well as those for the total T cells and B cells were applied to the cell count values obtained with TruCount™ tubes (BD Biosciences; San Jose, Calif.) and used to determine the absolute cell counts for each cell population. In studies 1-4 CD38+NK, B, and T cell subsets were assessed at baseline with the labeled anti-CD38 antibodies AB79 or Ab19 (FIG. 1). Although TSF-19 binds to a different epitope the results were very similar and are therefore not presented separately. Processed samples were analyzed immediately.

### PK Model Development

During PK model development, one-, two-, and three-compartment model structures were investigated. The two-compartment model was clearly superior to the one-compartment model, as judged by goodness-of-fit (GOF) plots and a decrease in objective function value (OFV). Based on visual inspections of diagnostic plots, the introduction of a third compartment was not necessary to describe the data adequately. The bioavailability (F) was modeled using the logit transformation F=exp (PAR)(1+exp (PAR)), where PAR designates the model parameter, to ensure that the estimates are bounded between 0 and 1. The non-linear PK at low concentrations was modeled with the quasi steady state (QSS) approximation model of the target mediated drug disposition (TMDD) process (Gibiansky and Gibiansky (2009) Expert Opin. Drug Metab. Toxicol. 5: 803-812).

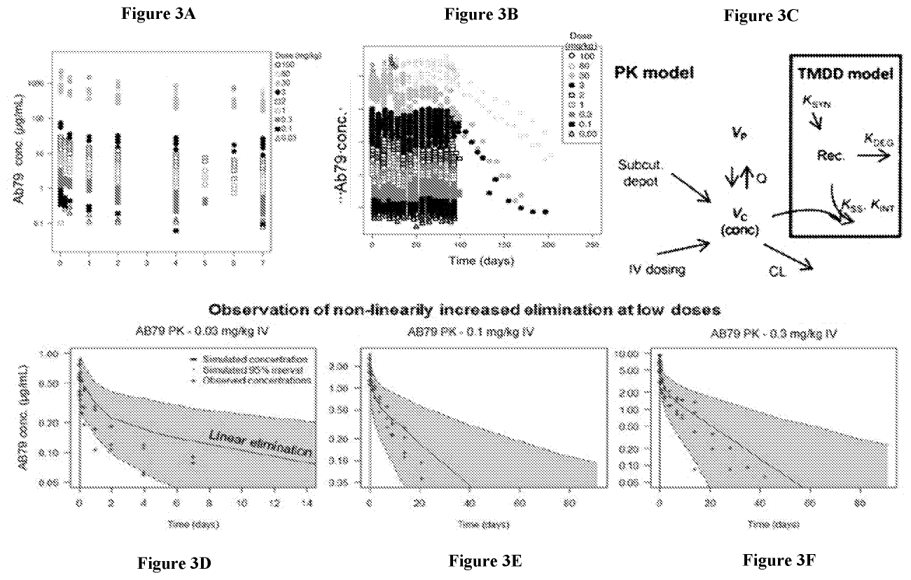

A schematic representation of the model is provided in FIG. 3C. For the QSS approximation we assume that the steady state concentrations of the free drug C, the target R and the drug target complex RC are established very quickly compared to all other processes. This implies that the binding process is balanced with the dissociation and internalization processes and that the following equation holds in the appropriate units: K_{ON}*C*R (K_{OFF}+K_{INT})*RC, where K_{ON} designates the binding rate constant and KO_{FF} the dissociation rate constant and KI_{NT} the internalization rate constant.

The between-subject variability (BSV) was investigated for all parameters and modeled with exponential models of the following type: PAR, TVPAR*e^{ETAPAR}ᵢ, where PARᵢ is the individual and TVPAR the typical parameter estimate and ETAPARᵢ is the estimate of the deviation of individual i. The ETAPARᵢ values were assumed to follow a normal distribution with mean zero. The residuals were described with a combined additive and proportional error model (Beal and Sheiner (1992) NONMEM User Guides, in University of California CA).

The following parameters were investigated to identify potential covariate effects on the PK of AB79: body weight, sex, dose, route of administration, and study.

### PK-PD Model Development

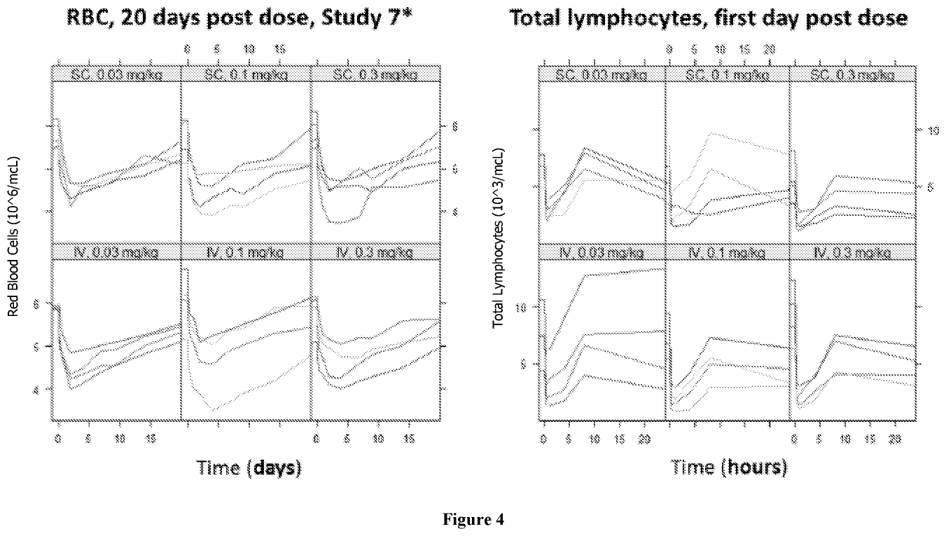

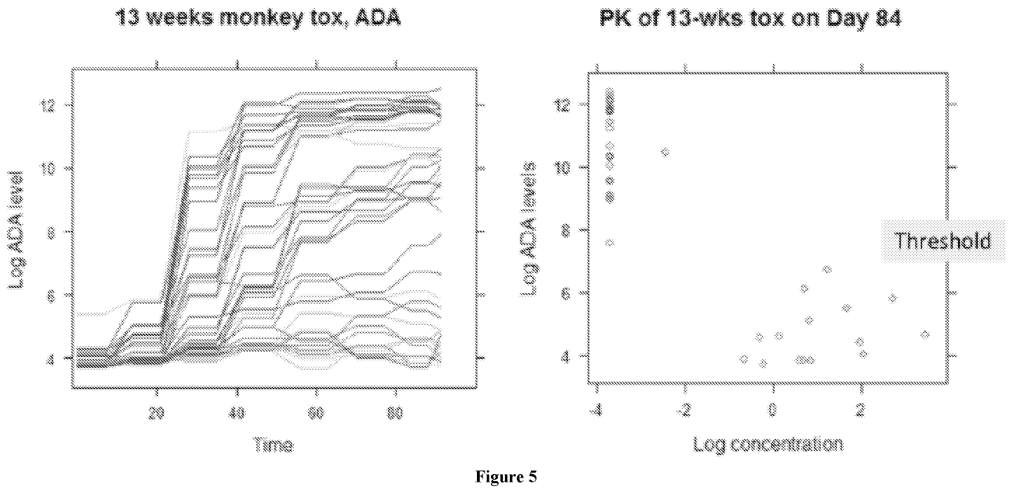

For each of the three cell types PK-PD model development was performed separately. Note that for model development measurements close to the drug administration (<8 hours post dose) were not utilized because they were influenced by a non-specific drug-independent effect potentially due to multiple blood samples taken over short amount of time (FIG. 4). The PK model and parameter estimates were fixed. Turnover, transit compartment and direct response models of various forms were tested (Friberg et al. (2002) J. Clin. Oncol. 20: 4713-4721; Mager et al. (2003) Drug Metab. Dispos. 31: 510-518). In the turnover models the drug effect was introduced on the cell elimination rate in form of an Emax type model with or without Hill factors. In our notation, an Emax model is a functionf of the drug concentration c of the following form: f(c)=EMAX*c^{H}(c^{H}+C50^{H}), EMAX denotes the maximal effect, C50 the concentration at which half of the maximal effect is achieved, and H the Hill factor. In the transit compartment model (TCM) the drug effect was introduced and tested on different positions: on the rate of proliferation, on circulating cells and on the third transit compartment. Also combinations of these effects and whether the data supports the presence of a feedback mechanism from the circulation to the rate of proliferation were also tested. In addition, Emax type direct response models with and without Hill factors were tested to describe the drug concentration-effect curve.

Random-effect parameters were introduced to estimate the between-subject variability on the baseline cell count, on the cell production rate (KIN), on transit time in the transit compartment model (MTT), on C50 and on EMAX. Individual mean baseline cell levels were provided in the data set (column BL). This was used as typical value in the model. A random effect parameter was added to enable the adjustment of the individual baseline estimate based on all measurements of the individual. The PD residuals were described with a proportional error model.

For model validation during the course of modeling (PK and PK-PD) OFV, standard errors, GOF plots and individual prediction versus data plots were used to assess the models and compare them to alternative ones.

The following software packages were utilized: NONMEM (Version 7.2), KIWI (Version 1.6), Berkeley Madonna (version 8.3.14), PSN (Version 4), and R (Version 3.3.0).

### Data Set Preparation

The data sets from the 8 monkey studies were collected, reorganized in a single format and merged in to three separate NONMEM readable PK-PD data sets. Each of the three data sets contained individual characteristics of the monkeys (study, ID, group, body weight, sex), the dosing information, the PK and either NK, B, or T cell data. For animals of the control groups only cell counts but no PK data were added to the data sets, assuming implicitly no serum levels of AB79. Time-resolved information about the antidrug immunogenicity status (ADA), namely TITER containing the quantitative measurement result and the 0/1-flag variable ADAF (ADAF=1 if ADA affects the concentration of AB79, ADAF=0 if it does not), was added in separate columns to each observation. ADA titers were measured with different method specifications in the different studies and, therefore, between studies the values are quantitatively not directly comparable. To utilize the ADA information in a consistent manner across all studies we applied the following procedure for each animal separately: ADA titers that increased at time points later than 7 days over the initially measured levels were considered ADA-positive and flagged in the data set (ADAF=1). If a sample at one time point was flagged ADA-positive all samples that were taken after that time point were also flagged ADA-positive in this animal regardless of the measured titer. ADA positive observations were not used for parameter estimation during model development. Note that also PD measurements from sampling time points of ADA affected PK concentrations were flagged with ADAF=1.

For the cell count data, the individual baseline values for each cell type (NK, B, and T cells) were calculated as mean value of all available predose measurements of a given animal. In most studies, this was a single measurement. The baseline value of each animal was then added as an observation at the time of the first dosing event (TIME=0) and as a constant value in column BL to each observation of the respective animal. Based on this baseline value the percent of baseline for each observed cell count was calculated and added to the data set.

### Scaling of Monkey PK Parameters

The final PK and PK-PD models were used as starting point to simulate PK and PK-PD profiles for the first in human clinical trial. Comparative analyses of data from therapeutic monoclonal antibodies have shown that PK parameters derived from studies in monkeys can be scaled to predict human PK profiles with acceptable accuracy (Han and Zhou (2011) Ther. Deliv. 2: 359-368). The publication indicated that using a fixed exponent of 0.85, human clearances of monoclonal antibodies can be predicted reliably. Consequently, this relationship was applied to scale human clearance parameters (CL, Q), whereas volume parameters (VC, VP) were scaled using a direct relation between body weights (BW):

$${CL}_{human} = {CL}_{animal} \cdot {( \frac{{BW}_{human}}{{BW}_{animal}} )}^{0.65}$$

$${V}_{human} = {V}_{animal} \cdot \frac{{BW}_{human}}{{BW}_{animal}}$$

### Results

### Pharmacokinetics of AB79

The PK data set was pooled from all 8 studies in healthy monkeys excluding the placebo groups (Table 3). In total, the set contained data from 140 animals, 58 of which were male and 82 female. The body weights of the studied animals ranged from 2.1 to 4.7 kg and the doses ranged from 0.03 to 100 mg per kg body weight (mg/kg). In one group of study 7 and three groups of study 8 doses of 0.03, 0.1, 0.3 and 1 mg/kg were administered SC (15 animals in total). The pooled data set contained 2,199 measurable PK observations greater than LLOQ (FIG. 3A, 3B). In parallel to AB79 concentrations ADA was assessed. 229 PK observations were found to be affected by ADA (FIG. 5). The PK was most densely sampled after the first dose and even in the long term toxicology studies most animals were terminated before Day 98. Only study 4 included recovery groups and we could only gather PK data from 4 animals, 2 from the 80 mg/kg group and one from each of the 30 mg/kg and 3 mg/kg groups (FIG. 3B).

Initially, for each of the monkey studies PK analyses were performed using standard non-compartmental techniques (NCA). Based on the single dose studies (IV bolus injection or 30 minute IV infusion) the volume of distribution during the terminal phase (Vz) was calculated to range from 64 to 116 mL/kg, the clearance from 6.04 to 14.7 mL/kg/day, and the terminal elimination half-life (T½) from 4.75 to 11.2 days. Area under the concentration time curve (AUC) and maximal concentration (Cmax) values were found to increase proportionally with dose over a wide range. Only the PK profiles of the lowest dose groups (<1 mg/kg, FIG. 3D-3F) provide evidence for non-linearly augmented clearance at concentrations below 0.5 μg/mL likely caused by target-mediated mechanisms (TMDD) (Kamath (2016) Drug Discov. Today Technol. 21-22: 75-83). Based on the data of all monkey studies excluding the two lowest dose groups (dose>0.3 mg/kg) a linear 2-compartment model was constructed. When the PK of the lowest dose groups was simulated and overlaid with the measured concentrations it was evident that the linear model over predicts the concentrations (FIG. 3D-3F).

The available PK data after single dose SC administration revealed that Cmax was 70-80% lower in the SC versus IV groups of the same dose and that the AUCs were comparable. No differences in PK parameters between male and female monkeys were observed. The results of these initial analyses were used as the starting point for model development.

### PK Model Development

Model development started with single IV dose data and then the initial model was gradually extended utilizing more complex data. Similar to other therapeutic antibodies, the PK grossly follows a linear 2-compartment model (Kamath (2016) Drug Discov. Today Technol. 21-22: 75-83). The non-linear elimination component (TMDD) describing the accelerated clearance at low concentrations was modeled with the quasi steady state (QSS) approximation (Gibiansky and Gibiansky (2009) Expert Opin. Drug Metab. Toxicol. 5: 803-812). The assumption that the drug-target association process is much faster than the processes of drug dissociation, distribution and elimination, and of target and drug-target complex elimination leads to the simplified TMDD model (FIG. 3, Table 4). The amount of data at low concentrations was relatively small such that all the parameters were not estimated in asingle estimation run of the software program. Therefore, the parameters of the TMDD model were first estimated by focusing on the data of the low single dose studies 7 and 8. The resulting TMDD parameter estimates were then kept fixed during the final estimations on the entire data set (Table 4).

TABLE 4
Population PK Modeling Results, Parameter Estimates
And Standard Errors In Percent (% SEM)

| | | | Interindividual Variability/ | |
|---|---|---|---|---|
| | Final Parameter Estimate | | Residual Variability | |
| Parameter | Typical Value | % SEM | Magnitude | % SEM |
| F | 0.227 | 121 | NE | — |
| K_{A} (L/day) | 0.399 | 20.5 | 42.1% CV | 53.8 |
| CL (L/day) | 0.0187 | 5.17 | 42.9% CV | 20.1 |
| V_{C} (L) | 0.141 | 3.23 | 19.8% CV | 22.5 |
| Q (L/day) | 0.127 | 14.7 | NE | — |
| V_{P} (L) | 0.127 | 6.45 | 39.4% CV | 20.8 |
| K_{INT} (1/day) | 0.1 | FIXED | 49.3% CV | 36.7* |
| K_{SS} (1/day) | 5.68 | 38.7* | NE | — |
| K_{SYN} (u/L/day)^{$} | 0.04 | FIXED | NE | — |
| K_{DEG}(1/day) | 0.00452 | 30.1* | NE | — |
| ROUT on V_{C} | −0.697 | 6.51 | NE | — |
| RV add | 3.17E−04 | 21.2 | 0.0178 SD | — |
| RV prop | 0.0677 | 1.58 | 26.0% CV | — |

Minimum value of the objective function = 5748.412
^{$}K_{SYN} is the synthesis rate of the receptor CD38. Since actual concentration measurements or information about the in vivo synthesis or degradation rate of CD38 were not available, “u” was used as unit for a certain unknown amount of CD38.
*The estimates and standard errors for the TMDD parameters were gained from a separate run that focused on the data of the low dose groups, and were then fixed for the final estimation of the other PK parameters. NE: Not Estimated.

Estimates for the absorption parameters KA and F were obtained when the data of the SC groups was added. All SC data came from four lower single dose groups from studies 7 and 8. These lower doses (≤1 mg/kg) covered the clinically relevant range but may limit the generalizability of the parameter estimates for higher doses.

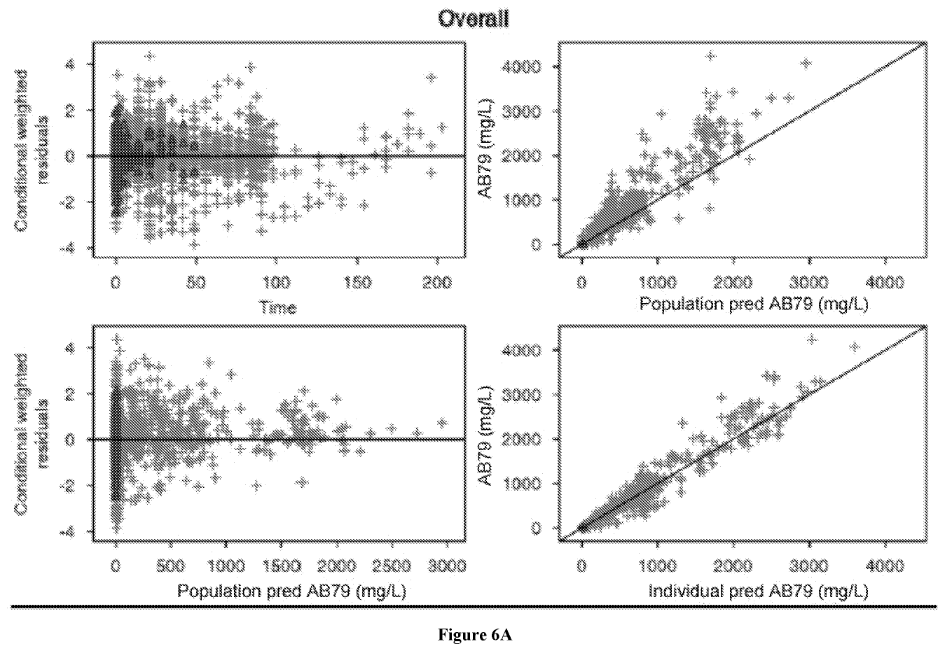

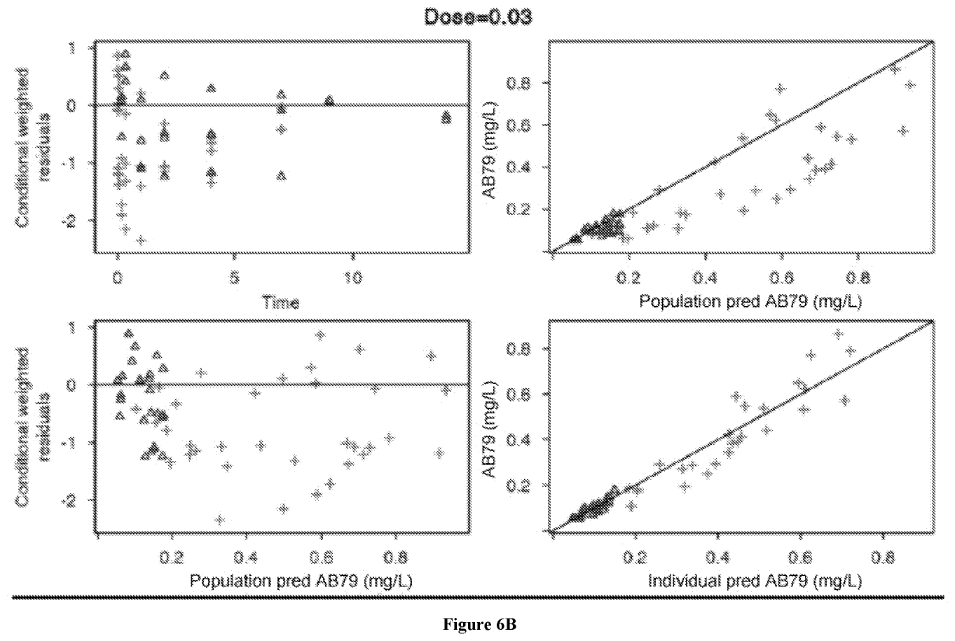

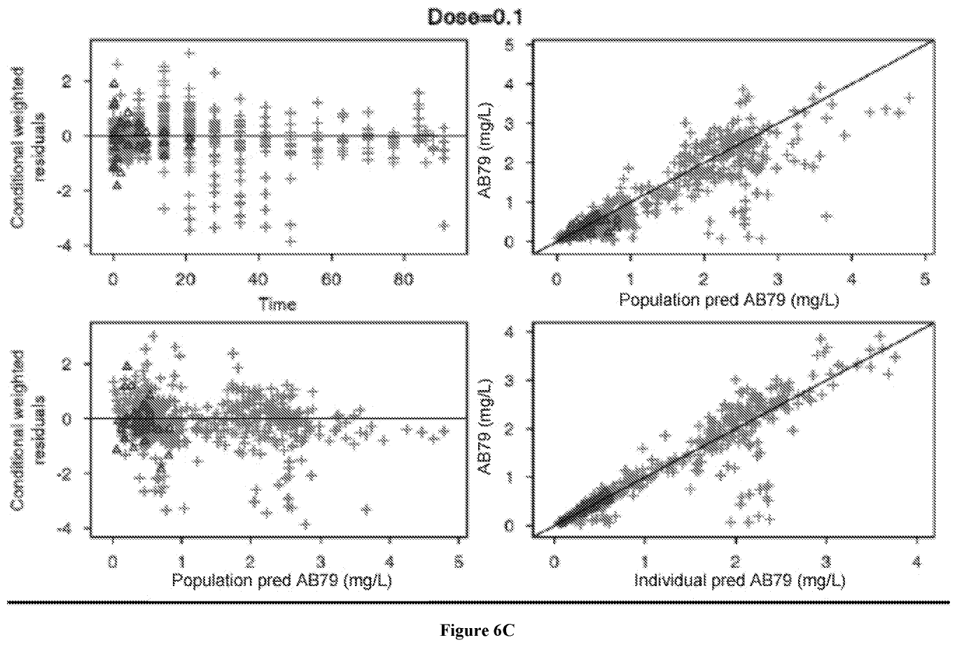

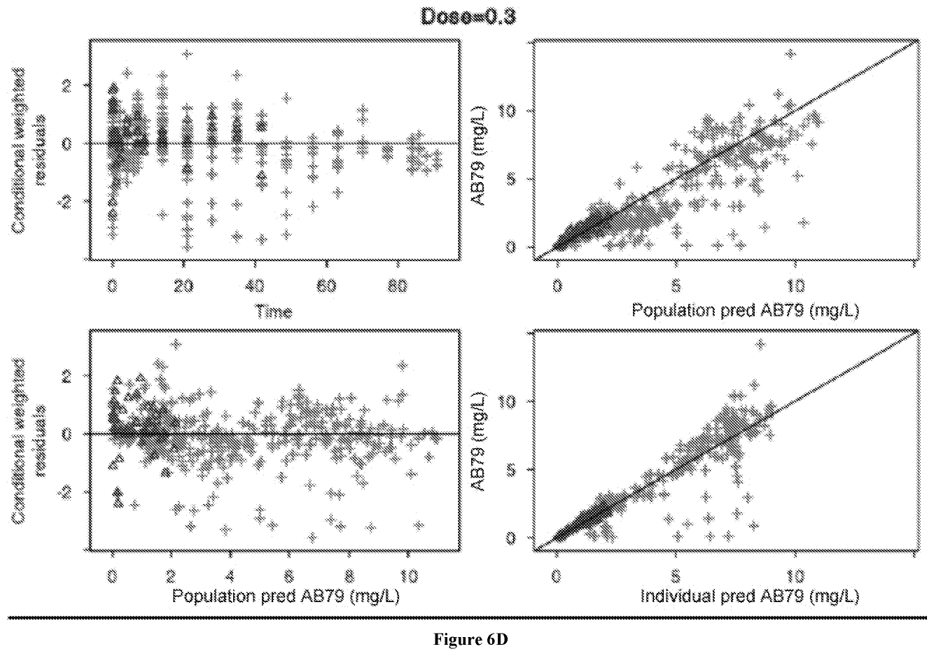

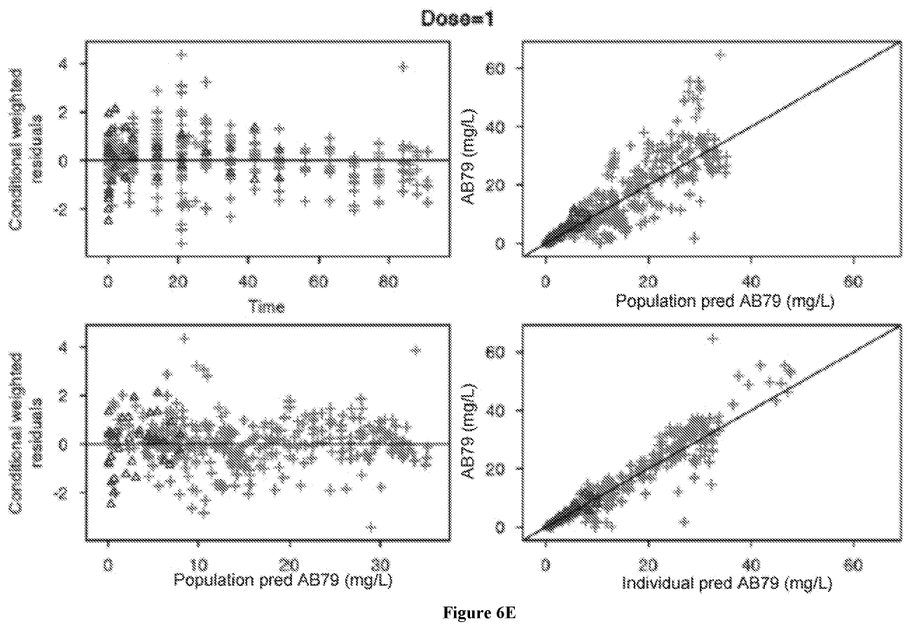

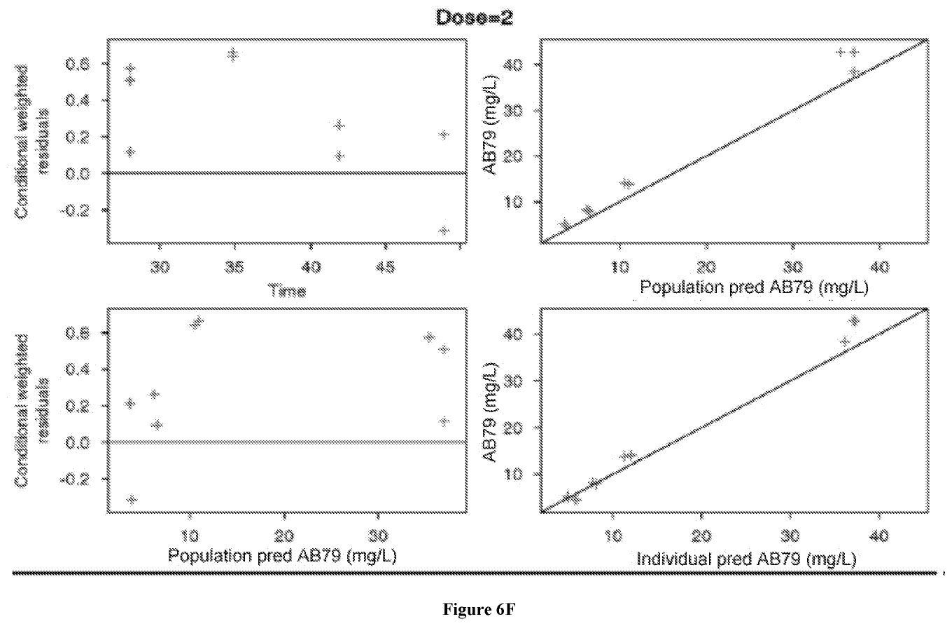

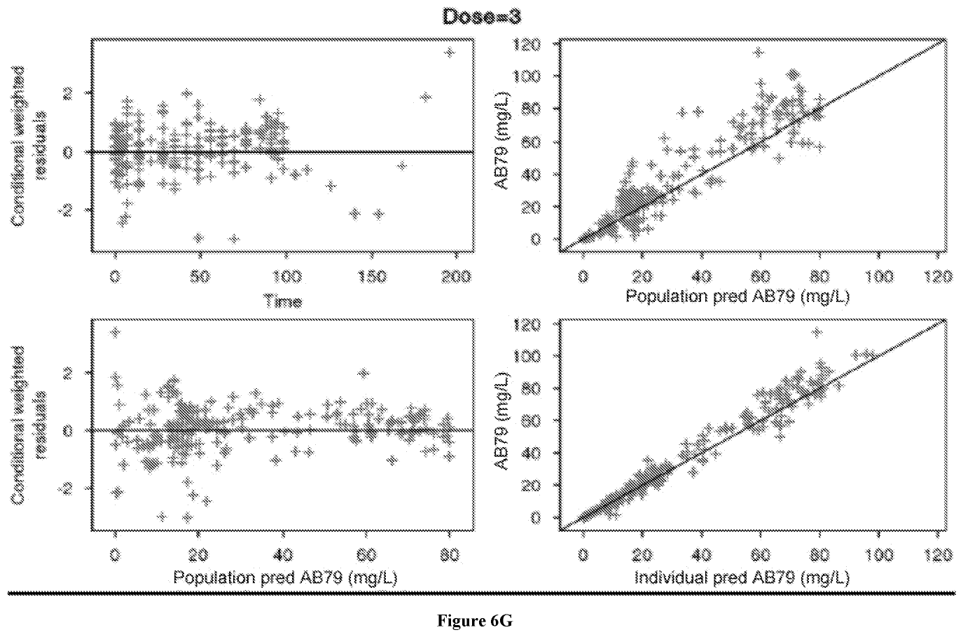

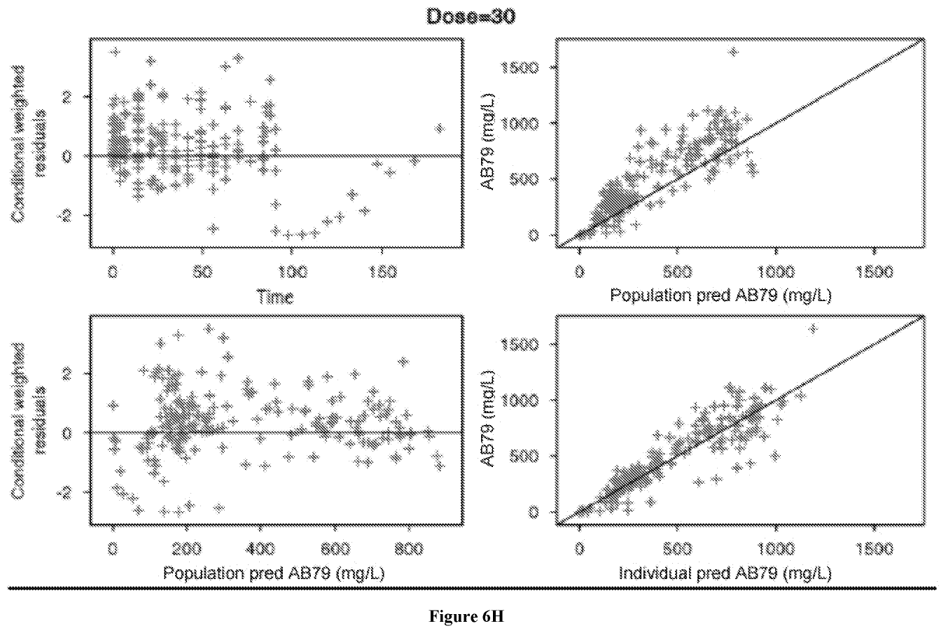

The between subject variability (BSV) for the PK parameters was described with exponential models. Absorption rate (KA), clearance (CL), and peripheral volume of distribution (V) have an estimated BSV of about 40% and the central volume of distribution (Vc) of about 20% (Table 4). Covariate analysis identified an effect of the route of administration on Vc. The typical value for Vc was 0.141 L if administered IV and 0.043 L (ca. 70% smaller) if administered SC. Other significant covariate effects were not identified. Because of the limited amount of data at low concentrations the between subject variability and the individual predictions of the TMDD parameters were only estimated for the internalization rate K_{INT} (BSV: 49%). Model evaluation based on residual errors, OFV, standard errors, GOF plots and individual curve fits corroborated that the final model adequately described the PK of AB79 in healthy monkeys (Table 4, FIG. 6).

### Pharmacodynamics

The level of AB79 binding on human and monkey blood NK cells, T cells and B cells was compared by flow cytometric analysis. As shown in FIG. 7, monkey lymphocytes had CD38 expression levels, based on AB79 molecules of equivalent fluorescence (MOEF), that was slightly lower compared to their human counterparts but with a similar relationship between cell types, e.g., CD38 expression on NK cells>B Cells>T cells. These data support the use of this non-human primate species as a relevant model to help predict AB79's potential for PD activity in humans.

For the in depth quantitative analyses of the relationships between drug exposure (PK) and the extent and duration of cell depletion (PD) we compiled data sets from PK concentrations, NK cell, B cell and T cell counts of all 8 monkey studies including the placebo treated animals, where available (Table 3). The initial characterization of the data set showed that at baseline, T cells had a median value of 3,732 cells per μL (interquartile range (IQR): 2,881-5,176) and were the most abundant lymphocyte subtype as compared to B cells with 1,279 cells per μL (IQR: 860.8-1,890) and NK cells with 685 cells per μL (IQR: 482.8-970.1). Baseline CD38 expression on these cell populations was assessed in studies 1-4 (Table 3, n=67). 86.7% (SD 11.3) of NK cells expressed CD38 with smaller variability. In contrast, 58.7% (SD 27.0) of B cells and 34.5% (SD 24.5) of T cells expressed CD38 with larger variability.

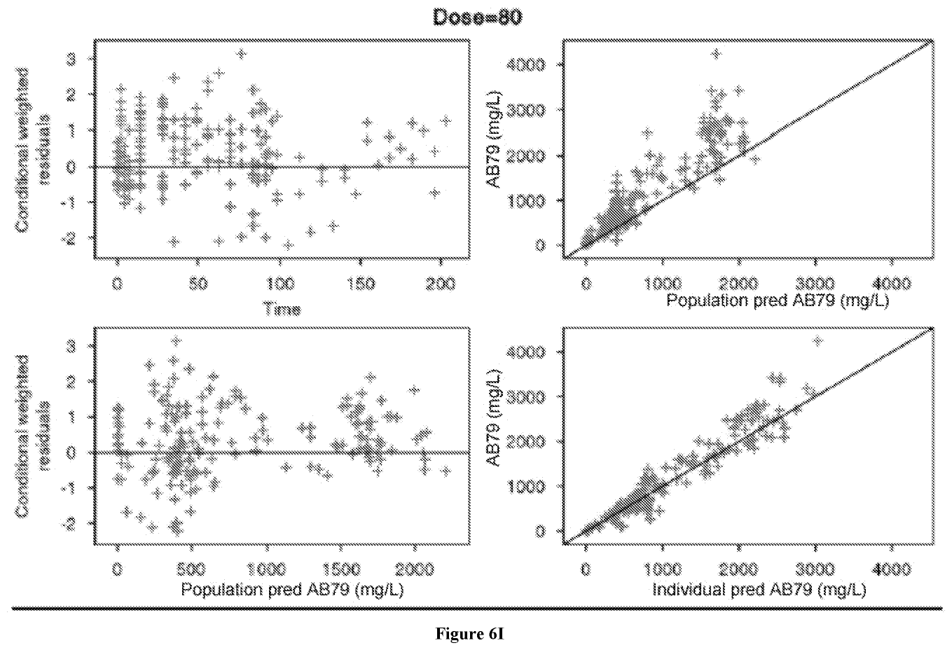

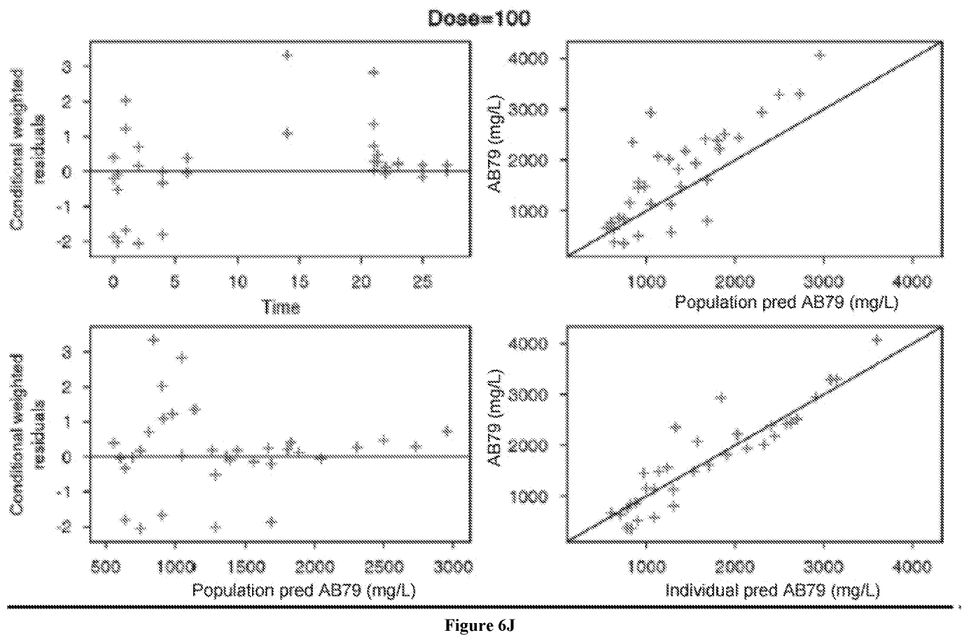

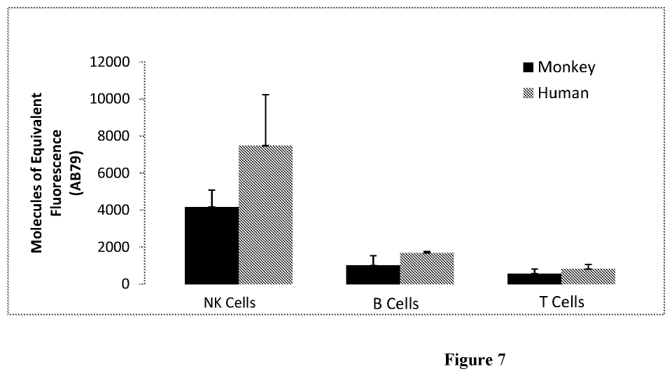

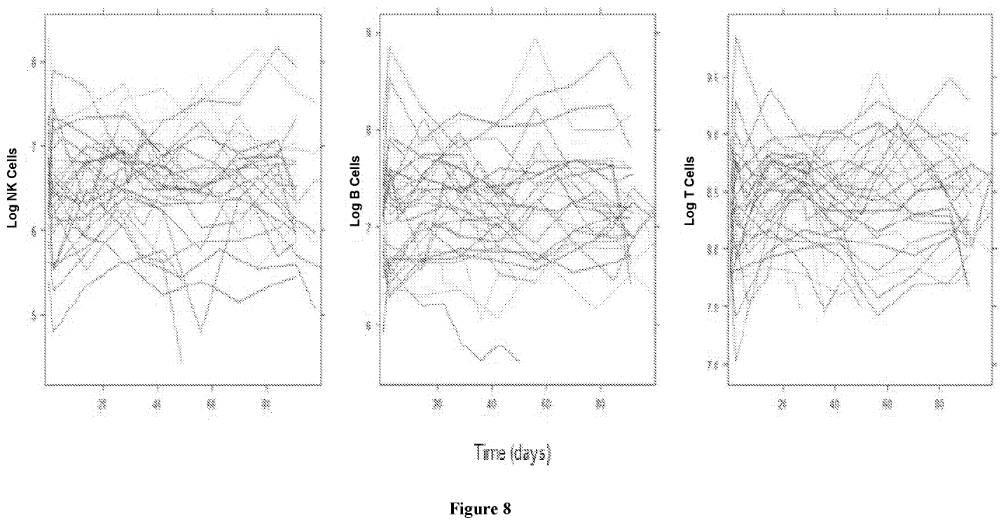

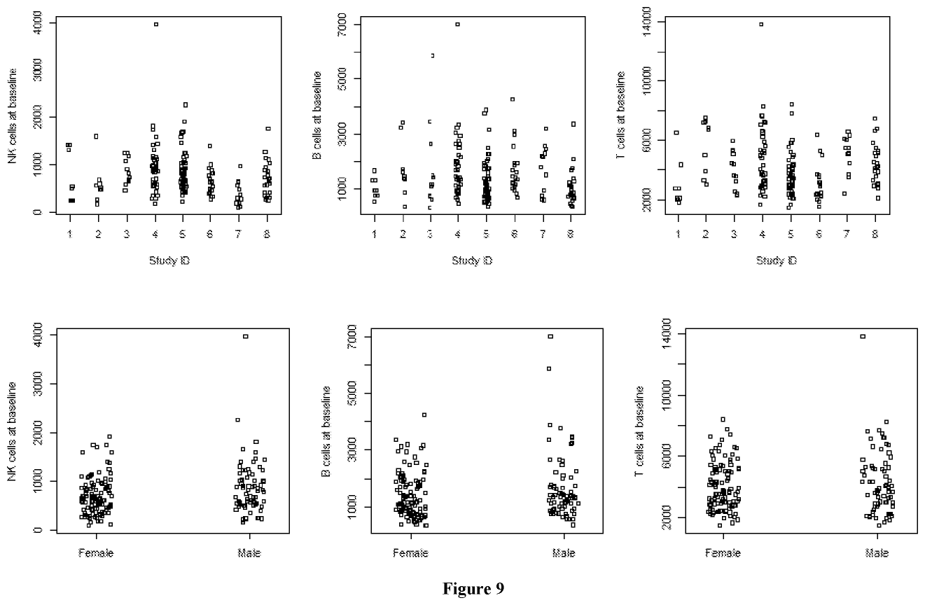

Data from the placebo treated animals showed that the average number of each of the cell types varied over time between individual animals more than one would expect from the variability within one individual (FIG. 8). For example, the average coefficient of variation of B cell counts of the individual placebo curves was 27% but the individual average B cell levels ranged from 436.6 to 4,389. In addition, there were also differences between average baseline lymphocyte numbers from male and female animals and from animals of different studies adding to the variability (FIG. 9). Based on these results each post-treatment cell count was calculated relative to its individual baseline value in percent, rather than the absolute cell numbers at each time point. For example, a value of 33% means that in the sample cell count was ⅓ of the baseline cell count. This provided standardized values that could be compared across the entire data set.

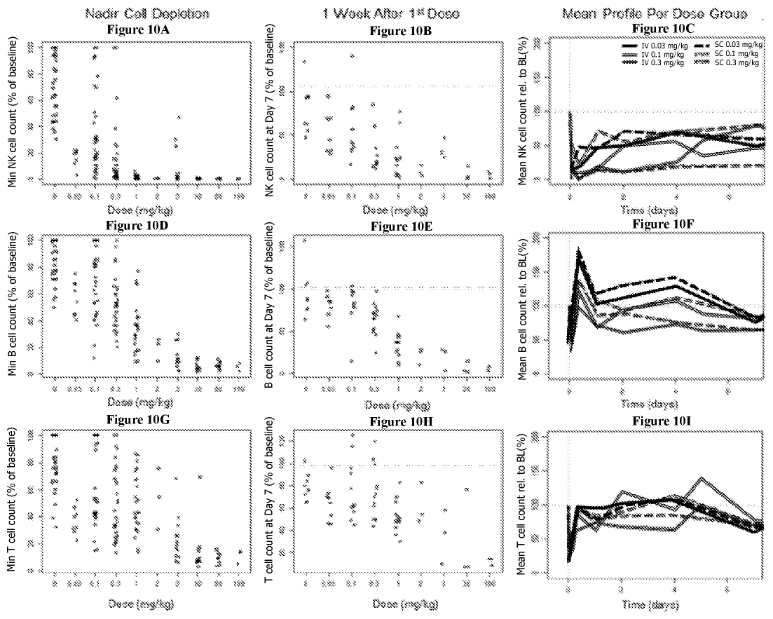

The rapid onset of depletion of AB79 binding cells suggests that the initial blood concentration drives the decrease in lymphocyte counts (FIG. 10). At IV doses of 0.3 mg/kg AB79, the median maximal effect on NK cells was depletion of 93.9% (i.e., 6.1% of baseline cell counts remaining). At 0.1 mg/kg, the peak depletion was 71% (29% of baseline remaining). At doses>0.3 mg/kg, NK cells were nearly completely depleted in the blood compartment (Nadir (range): 1.06% of baseline (0.17, 6.23); FIG. 10A). After a single dose of 0.3 mg/kg it took approximately 7 days for the NK cells to recover to an average of 50% of baseline, albeit the kinetics of the recovery were highly variable between individuals (FIG. 10B, 10C). In concordance with these results, NK function was also tested in a subset of animals in study 7 (n=3/group; Table 3). This experiment showed a dose-dependent reduction with minimal changes in blood NK activity at 48 hours post-treatment in animals treated with 0.1 mg/kg AB79 (% lysis at 100:1 effector: target ratio±SD; 44.5%±23.6% vs. 41.4%±25.8%) and almost complete loss of NK activity in animals treated with 1.0 mg/kg (% lysis at 100:1 effector: target ratio SD; 37.4% 10.3% vs. 6.8%±12.5%). NK cell function showed recovery at 57 days, the next time point measured (% lysis at 100:1 effector: target ratio SD; 16.0%±11.9%).

B cells and T cells were depleted to a lesser extent as compared to NK cells, which is consistent with their lower CD38 expression levels (FIG. 7). At 0.3 mg/kg IV AB79, for example, B cells had a median maximal level of depletion to 45% of baseline, and T cells were depleted to 43% of baseline (FIG. 10D, 10G). At this dose level, a 50% reduction from baseline of B cell counts was not achieved in all animals. Only at the highest doses of ≥30 mg/kg were the B cells almost completely depleted (FIG. 10D). T cells were depleted to an extent similar to B cells but the recovery was faster (FIG. 10G-I).

In the two studies 7 and 8, the IV and SC dosing (FIG. 10C, 10F, 10I) was compared. There were no obvious differences in cell depletion between the routes of administration. At the lower doses (study 8) a sustained (>24 h) cell depletion of 50% below baseline values was only seen in the NK cell population and not with T and B cells; although all cells showed specific cell depletion at early time points. The timing of the onset of NK cell depletion appeared similar between dose groups regardless of the route of administration and the duration of depletion was dose-dependent. Cell recovery in all test groups was seen by Day 57.

### PK-PD Models

Separate PK-PD models were developed to describe the effects of AB79 exposure on NK, B, and T cells. During PK-PD modeling the PK parameters were kept fixed to the estimates of the final PK model and a variety of PD models were tried (see Materials and Methods for detail). The NK cell population in the peripheral blood was adequately described with a turnover model and the depleting drug effect was linked via the PK concentration with an Emax type model to the rate of depletion. In this model the EMAX represents the maximum rate of additional NK cell depletion and the C50 the concentration at which the rate of additional NK cell depletion is half-maximal. The structural PK-PD model for NK cells was of the following form:

$$\frac{dNK}{dt} = {K}_{IN} - {K}_{OUT} \cdot NK - NK \cdot \frac{EMAX \cdot c}{C 50 + c}$$

In the formula NK represents the actual NK cell count, K_{IN} the production rate and K_{OUT} the elimination rate when no drug is present. Note that with the given baseline measurement BL K_{OUT} is defined by the equation K_{OUT}=K_{IN}/BL. c represents the AB79 concentration in the central compartment. When all parameters were estimated at once the software program did not produce stable results. The individual estimates of KIN, EMAX and C50 were highly correlated. Moreover, due to limited differentiation between the maximal effects of the different doses (see previous section) and the large interindividual variability, accurate estimates of all parameters could not be expected. In a series of estimations, one or two of the three parameters KIN, EMAX and C50 were fixed to different values and estimated the others. A stable run and reasonable goodness of fit with a fixed KIN of 10,000 and an EMAX of 322 was achieved. The typical C50 estimate was 29.0 μg/mL (Table 5). In addition, the sensitivity of the selected KIN and EMAX values was tested by choosing different combinations of higher and lower values. The between subject variability was large with 113% for the NK production rate K_{IN} and with 149% for the C50, which is in accordance with the large individual differences at baseline and between treated animals. The model was evaluated based on residual errors, OFV, standard errors, GOF plots and individual curve fits (Table 5, FIG. 4).

TABLE 5
PD Modeling Results, Parameter Estimates
And Standard Errors In Percent (% SEM)

| | | | Interindividual Variability/ | |
|---|---|---|---|---|
| | Final Parameter Estimate | | Residual Variability | |
| Parameter | Typical Value | % SEM | Magnitude | % SEM |

NK Cells

| KIN | 10000 | FIXED | 113% | CV | 19.2 |
|---|---|---|---|---|---|
| (count/day) | | | | | |
| C50 (μg/mL) | 29.0 | 18.8 | 149% | CV | 25.1 |
| EMAX | 322 | FIXED | NE | | — |
| Baseline (NK | NE | — | 28.3% | CV | 20.8 |
| cells)* | | | | | |
| NK cells | 0.291 | 2.42 | 53.9% | CV | — |
| residual | | | | | |

B Cells

| MTT (day) | 8.48 | 15.4 | 135% | CV | 17.4 |
|---|---|---|---|---|---|
| C50 (μg/mL) | 19.5 | 7.58 | NE | | — |
| EMAX | 2.37 | FIXED | NE | | — |
| Baseline (B | NE | — | 24.1% | CV | 10.7 |
| cells)* | | | | | |
| B cells residual | 0.136 | 2.20 | 36.9% | CV | — |

T Cells

| C50 (μg/mL) | 11.86 | 7.267 | NE | | — |
|---|---|---|---|---|---|
| EMAX | 0.4656 | 6.578 | 69.46% | CV | 29.50 |
| Baseline (T | NE | — | 29.08% | CV | 15.50 |
| cells)* | | | | | |
| T cells residual | 0.1343 | 2.406 | 36.65% | CV | — |

*For each individual animal the typical baseline value was calculated as average of all predose measurements;
NE: Not Estimated

The transit compartment model was superior to direct response or turnover models to describe AB79 induced B cell depletion. Four transit compartments turned out to be adequate and the drug effect was described with an Emax type model on the depletion rate. Similar to the NK cell depletion model, the EMAX represents the maximum rate and the C50 the concentration at which the rate is half-maximal. Thus the structural PK-PD model for the B cells is given by the following five equations:

$$\frac{{dTR}_{1}}{dt} = {K}_{PROL} - {K}_{TR} \cdot {TR}_{1}$$

$$\frac{{dTR}_{i}}{dt} = {K}_{TR} \cdot {TR}_{i - 1} - {K}_{TR} \cdot {TR}_{i} , for i = 2 , 3 , 4 \\ \frac{dB}{dt} = {K}_{TR} \cdot {TR}_{4} - {K}_{CIRC} \cdot B - B \cdot \frac{E MAX \cdot c}{C 50 + c}$$

TRᵢ (i 1-4) represent the four transit compartments. K_{TR}, K_{PROL} and K_{CIRC} are defined by the following equations K_{TR}=K_{PROL}=K_{CIRC}=4/MTT, where MTT is the mean transit time (Friberg et al. (2002) J. Clin. Oncol. 20: 4713-4721). B represents the B cell count in the blood and c the AB79 concentration in the central compartment.

With a fixed EMAX of 2.37 the typical C50 was 19.5 μg/mL and the typical mean transit time (MTT) was 8.48 days (Table 5). The delay of the maximal effect relative to the maximal AB79 concentration was well captured. The model indicates that AB79 primarily affects circulating B cells. No additional effects and no feedback loop on progenitor cells were necessary to describe the available monkey B cell data. Between-subject variability on MTT of 135% and on the baseline B cell levels (BASE) of 24.1% indicates large individual differences between animals.

The drug induced depletion of T cells with a rapid recovery was adequately described with a direct response model: T(c) BL_{T}*(1−EMAX*c/(c+C50)), where T represents the actual T cell count, BL_{T} the T cell count at baseline and c the AB79 concentration in the central compartment. The typical C50 was estimated to be 11.86 μg/mL and the typical EMAX was 0.47, indicating that in this case only about half of the T cells can be depleted by AB79 (Table 5). Note however, that the between subject variability on EMAX was nearly 70%. In this model, different from the NK and B cell depletion models, the C50 represents the concentration at which the depletion of T cells was half-maximal.

As for the NK cells, model evaluation of the final PK-PD models for B and T cells based on residual errors, OFV, standard errors, GOF plots and individual curve fits corroborated that they adequately described the available monkey data (Table 5, FIG. 4).

### Simulation of Human PK and Cell Depletion

The monkey PK and PK-PD models were used as starting point for the model-based simulation of human PK and cell count data to support the design and to justify the selected doses for the first in human (FIH) clinical trial in healthy volunteers. To this end, it was assumed that the model structures including TMDD derived from the monkey data also describe the main features of the human PK and the ensuing lymphocyte depletion. To obtain predictions for the human PK parameters we scaled the estimates of the following monkey PK parameters: central and peripheral volume of distribution (V_{C}, V_{P}), and clearance (CL) and intercompartmental clearance (Q) with a straight-forward approach for monoclonal antibodies (Han and Zhou (2011) Ther. Deliv. 2: 359-368). AB79 is a fully human monoclonal antibody and, therefore, we expect less immunogenicity in humans than that observed in monkeys. Consequently, for modeling and simulation we excluded ADA-positive samples from the data set.

Using the scaled model, exposure was simulated and NK, B, and T cell depletion profiles for single doses via a 2 hours infusion (IV) or via subcutaneous injection (SC) from 0.0003 to 1.0 mg/kg as planned for the FIH study (FIG. 11). According to the simulations, after an IV dose of 0.0003 mg/kg any observable drug induced effects on lymphocyte counts and not even measurable PK concentrations above LLOQ would not be expected. Due to the variability and due to the limited size of the dose groups it was assumed that the minimal detectable drug effect on NK cell counts would be a reduction of at least 10%. At doses of 0.01 mg/kg IV and 0.03 mg/kg SC, it was predicted that NK cells were to be depleted to less than remaining 90% of baseline.

At an IV dose of 0.3 mg/kg we predicted NK cell depletion to remaining 17% of baseline within 3 hours after the end of infusion and recovery to more than 50% after 11 days (FIG. 11). At the same dose the model predicts that B cells are maximally depleted to 67% of baseline after 2.5 days and T cells are immediately depleted to 86% of baseline. For the subcutaneous administration of the same dose of 0.3 mg/kg, the model predicted that it leads to less and later maximal depletion (nadirs relative to baseline: NK cells 37%, B cells 74%, T cells 94%).

These in vitro and in vivo preclinical studies demonstrate that the monkey is an appropriate animal model to study the pharmacology of AB79. Densely sampled PK and cell count data of NK, B, and T lymphocytes from eight monkey studies with diverse doses and dosing regimen provide a rich data source for a comprehensive and quantitative understanding of the relationships between AB79 dose, exposure, and cell depletion. The generated population PK and PK-PD models adequately describe the observed data and provide a powerful tool to predict exposure and lymphocyte depletion not only for future studies in monkey but also for clinical trials in human subjects.

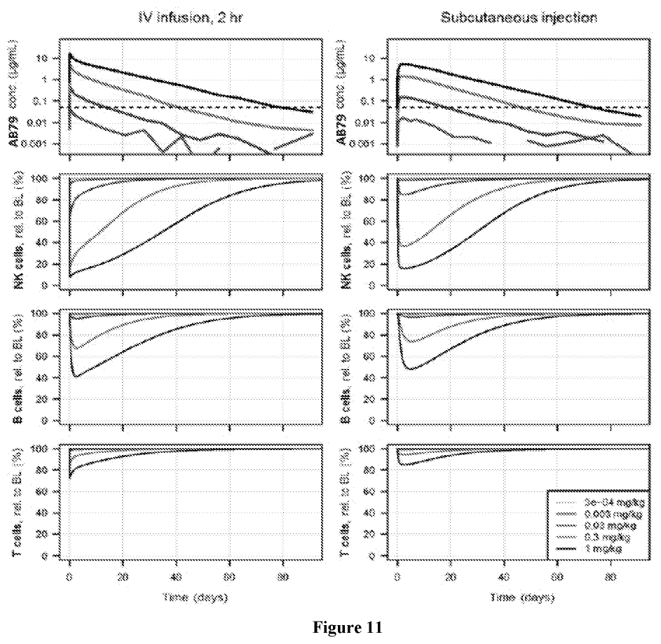

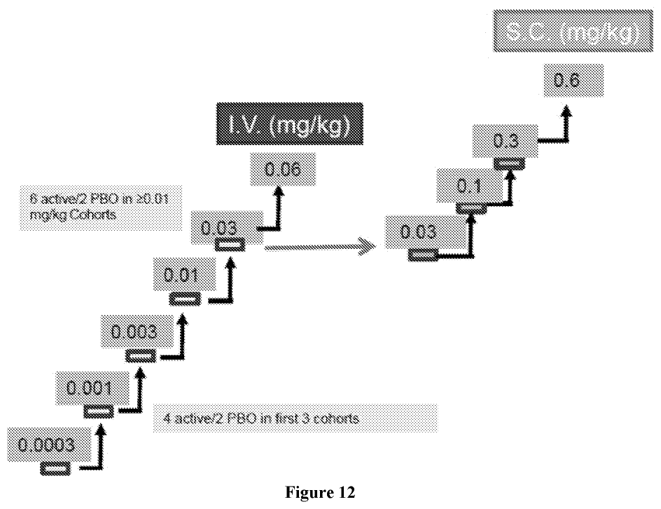

The first in human (FIH) single rising dose trial in healthy volunteers has been conducted (www.clinicaltrials.gov: NCT02219256) (FIG. 12). The intended pharmacological effect of AB79 is the depletion of activated lymphocytes. A profound and lasting depletion of lymphocytes (enhanced pharmacology), however, can lead to impairments of the immune system, which would not be tolerable for patients or healthy study participants. Therefore, a safe I.V. starting dose of 0.0003 mg/kg was chosen for the FIH trial.

The monkey data suggested that NK cell depletion was determined to be the most sensitive biological effect. The PK-NK simulation results helped to determine the minimal dose level of 0.01 mg/kg IV at which the most sensitive pharmacological effect (NK cell depletion) would be expected to be detectable in humans. The emerging data of the FIH trial revealed that the overall pattern of the dose-dependent and cell type specific depleting effects of AB79 are in accordance with the model-based predictions (manuscript in preparation). AB79 appears to be even more efficient than predicted. For example, at an IV dose of 0.03 mg/kg, NK cells in human subjects were depleted to remaining less than 10% of baseline. The median nadir (lowest depletion point) in monkeys at this dose was 20.0% (FIG. 10).

Three cytolytic anti-CD38 monoclonal antibodies (daratumumab, isatuximab and MOR202) are in clinical development for multiple myeloma (van de Donk et al. (2016) Immunol. Rev. 270: 95-112). Daratumumab (Darzalex™, givenasanintravenous infusion) was recently approved for multiple myeloma in the United States and for non-Hodgkin lymphoma in Europe. Unlike AB79, daratumumab does not cross react with monkey CD38. Therefore, a comparison of our results with AB79 in cynomolgus monkey with daratumumab was not possible. Moreover, multiple myeloma patients have high levels of CD38 positive malignant cells, which could require higher effective antibody concentrations for this cancer indication (de Weers et al. (2011) J. Immunol. 186:1840-1848).

It is however remarkable that daratumumab is approved at a weekly IV dose of 16 mg/kg in multiple myeloma, even though AB79 achieved complete depletion of peripheral NK cells at ca. 1 mg/kg and of B cells at ca. 3 mg/kg (FIG. 10).

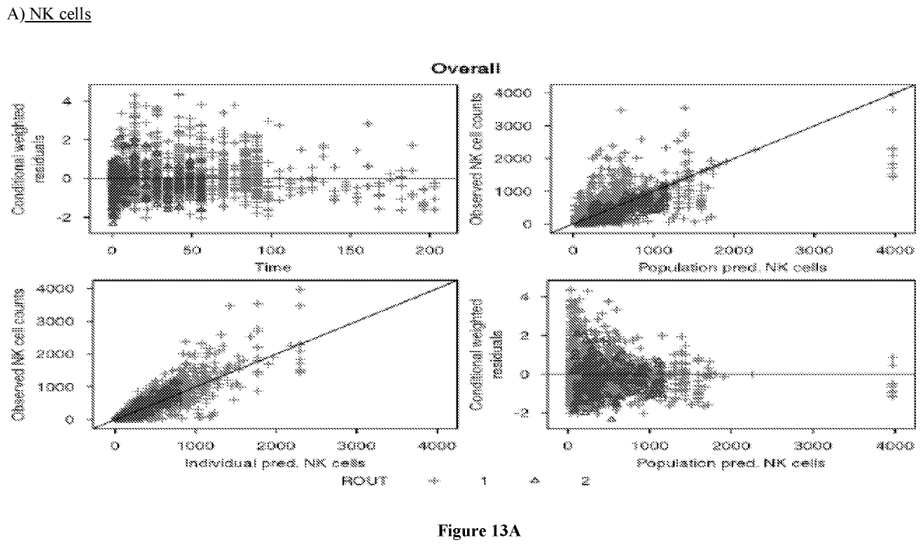

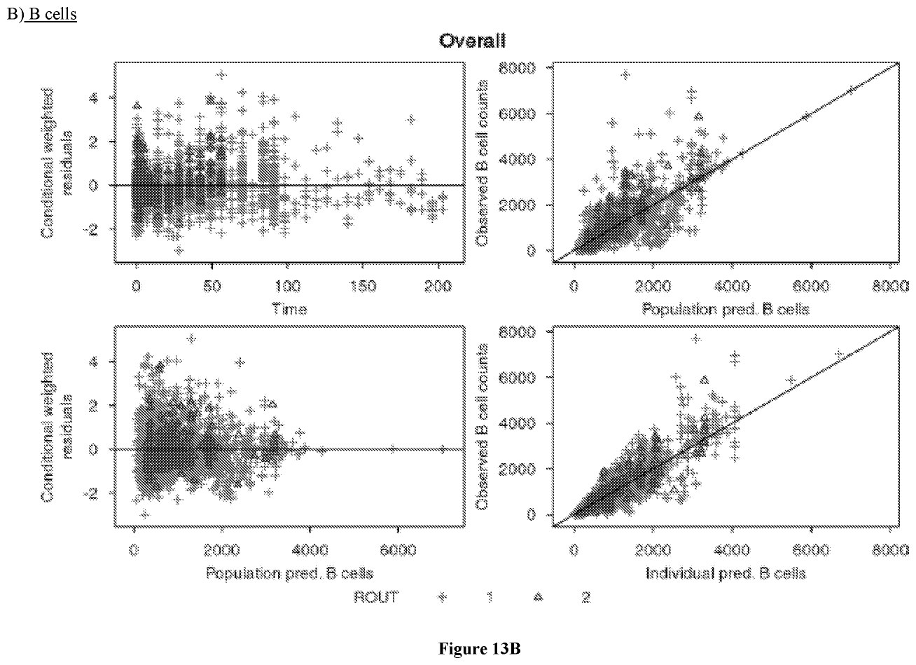

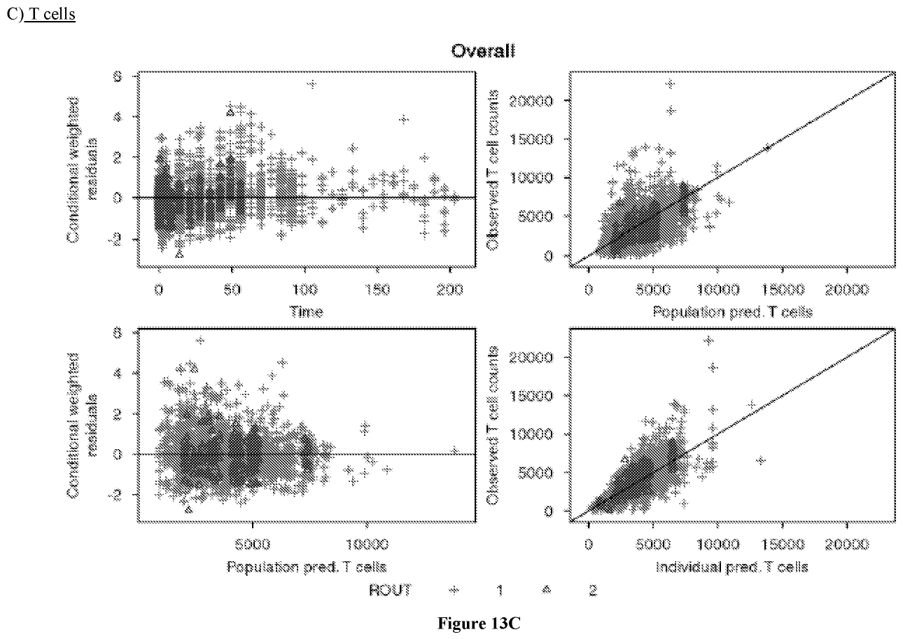

In spite of the rich database from 8 monkey studies, a number of limitations were recognized. AB79 effectively depletes NK cells even at the lowest studied dose of 0.03 mg/kg. At such low doses the PK quickly drops below the quantification limit of the bioanalytical assay, which prevented resolving the exposure-effect relationship at lower doses. Moreover, during preclinical development it was recognized that maximal cell depletion occurs shortly after the maximal drug concentration but the resolution of the early phase of depletion is technically limited by the overall sample number and potentially by non-specific cell depletion due to repeated blood collections (blood draw effect). The blood draw effect was observed as a transient pancytopenia characterized by depletion of cell types that do not bind AB79 and was not dose-dependent suggesting it was due to loss of blood volume as a result of multiple blood draws rather than any specific effects of AB79 (FIG. 13). Consequently, the power to accurately estimate the model parameters especially for NK cell depletion was limited and the typical values of KIN and EMAX required fixing to achieve stable and adequate estimation results.

The effect of AB79 on tissue plasma cells or plasmablasts could not be measured. However, like plasma cells and plasmablasts, NK cells have high levels of CD38 on their surface and cell depletion efficiency of a specific lymphocyte subset depended, at least in part, on the expression levels of CD38. Therefore, the cytolytic effect of AB79 on plasmablasts and plasma cells may be comparable to the effect on NK cells. At present, the information about long term effects of AB79 treatment in monkeys is limited. Only a small subset of animals in the 13-weeks toxicology studies was investigated in a recovery group over a longer period of time and most of the animals in all dose groups developed ADA. Moreover, the baseline values and depletion profiles of the different lymphocyte subsets were highly variable between individuals. Therefore, the long term effects of AB79 cannot be investigated in monkey and will have to be studied in humans.

With the emerging human data it will be interesting to compare human and monkey PK and PD data in detail. The construction of a PK model based on human data and a comparison to the monkey model will allow refining the TMDD model of AB79. Data generated in patient studies will provide insights regarding how AB79 mediated depletion of B lineage cells compares between RA and SLE patients and those of multiple myeloma patients and healthy subjects. The investigation of subject or disease related factors that may influence cell depletion efficiency in addition to CD38 expression levels is also important and could lead to a personalization of the treatment. In addition, a thorough head-to-head comparison of AB79 with daratumumab and/or the other CD38 antibodies in vitro and in vivo will reveal valuable information about the pharmacology of anti-CD38 antibodies and their optimal application.

The rich pharmacological data and the PK and PK-PD models enabled characterization of exposure-effect relationships in cynomolgus monkeys. The model-based analyses of NK, B, and T cells supported and quantified the finding that each of the blood lymphocyte subsets are depleted by the antibody at different rates and require different time spans to replete the blood compartment. The models proved to be excellent means for simulations of PK and PD data under different dosing scenarios in preparation of clinical trials.

### Example 2: CD38+ Cell Depletion By AB79

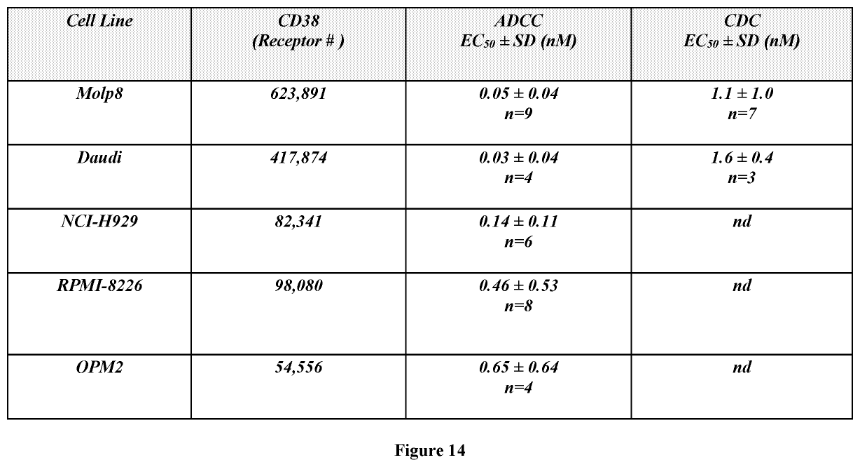

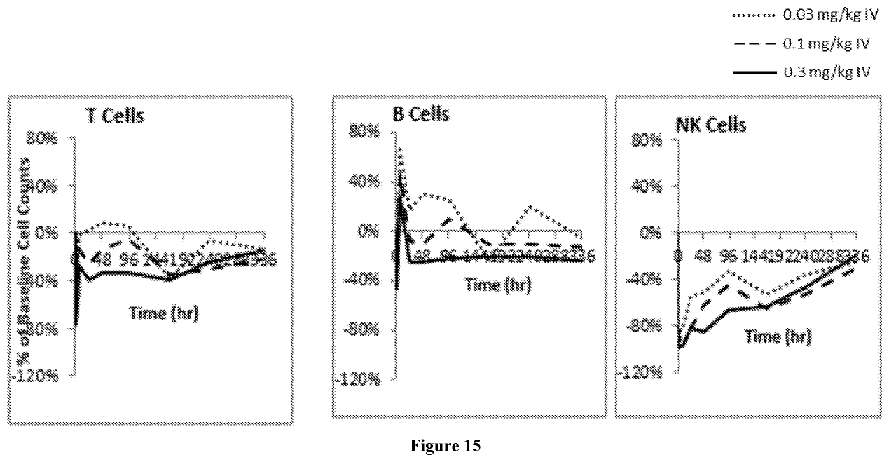

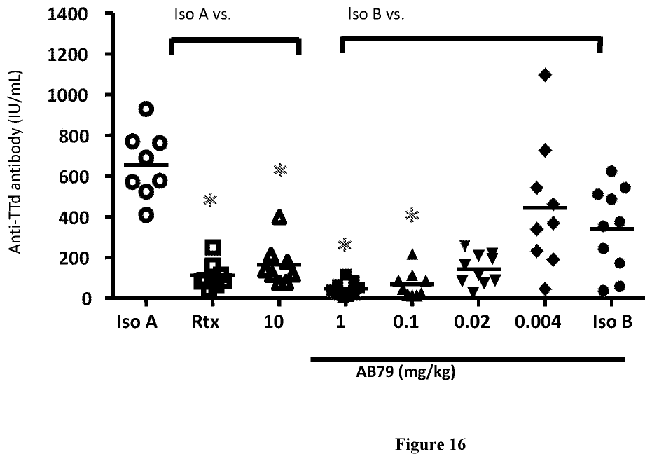

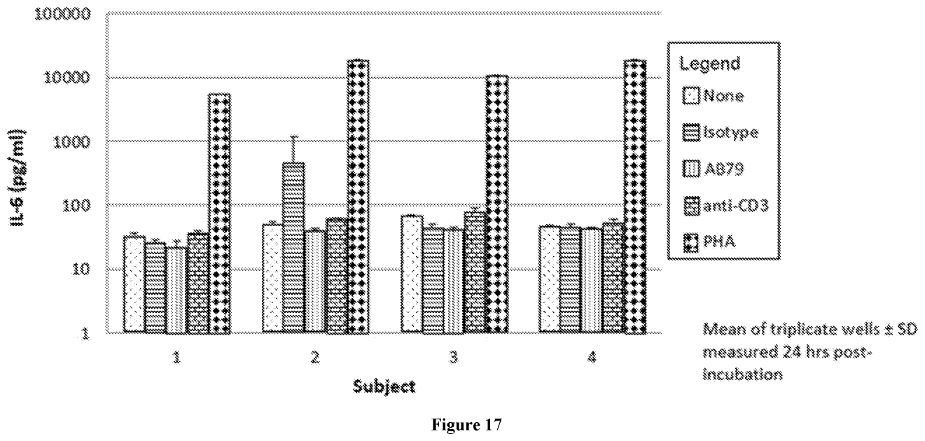

CD38 is a cADPR hydrolase expressed on human plasmablasts, plasma cells, NK cells and activated T and B cells, but is not on mature platelets or red blood cells, based on AB79 binding. In patients with rheumatoid arthritis (RA) and systemic lupus erythematosus (SLE), plasma cells, as well as activated B and T cells may be important contributors to disease. Unlike other B cell-selective therapies which target CD20 and do not directly deplete plasmablasts, which are CD20^{low/negative}, CD38 is expressed at high levels on plasmablasts and plasma cells making these cells a direct target of AB79. In vitro studies with human blood cells and cell lines showed that binding of AB79 to CD38 did not result in PBMC cytokine activation demonstrating that AB79 is not an agonist, as discussed below. Rather, AB79 mediated cell depletion of human B lineage cell lines by ADCC and CDC and in most cases cell lines with increased CD38 expression were more susceptible to cell lysis (FIG. 14). This is consistent with findings in healthy cynomolgus monkeys where the efficiency of depletion correlated with level of CD38 expression and AB79 dose level. NK cells, which express high levels of CD38, were depleted to a greater extent than CD20+ B cells and CD3+ T cells, which express less CD38 (FIG. 15). In vivo, AB79 potently suppressed the human B cell recall responses to antigen in a mouse adoptive transfer model (FIG. 16). Together these data support the further investigation of AB79 in autoimmune diseases.

Human PBMCs were treated with AB79 under multiple conditions and inflammatory cytokine release measured. The cynomolgus monkey was used to show the relationship of cell type-specific depletion and AB79 dose because AB79 cross reacts with monkey CD38, which shares 91% protein identity to the human protein. A second animal model, mice adoptively transferred human PBMCs, was used to determine if AB79 could target human antibody producing cells.

### AB79 Binds CD38 and Mediates ADCC and CDC

Receptor number was determined with the FIKIT (DAKO, cat #K0078) using mouse anti-human CD38 antibody (clone HIT2) and was calculated by converting mean fluorescence intensity (MFI) of the stained samples to a calibration curve generated from the MFI of 5 populations of beads bound with a defined number of antibody molecules. Absolute receptor # was calculated by subtracting isotype control (mouse IgG1) MFI from anti-CD38 antibody MFI.

CDC was evaluated by plating cell lines at 10,000 cells/well and adding AB79, control IgG or media. A 5-point dose-response curve (0.001-10 mg/ml) was typically performed. Rabbit complement (2-15 ul; #CL 3441 CedarLane Laboratories), was added to each well except control wells. CytoTox-Glo reagent (Promega, G7571/G7573) was used to detect cytotoxicity by luminescence. Tested groups: cells alone; cells+complement; cells+IgG control+complement; cells+AB79+complement. % CDC equation: % CDC=100-((RLU (test)/RLU (complement alone))×100).

ADCC was tested by plating 5000 target cells/well (T, cell lines) with 50 ml of AB79, control IgG, Triton X-100 (1%: Sigma Chemical) or media alone and 50 ml of human effector (E) PBMCs at a ratio of between 1:25 to 1:50 T:E cells. A 9-point antibody dose-response curve (0.000001-100 nM) was typically performed. Experimental lysis=PBMCs+cell line+antibody. Spontaneous lysis=PBMCs+cell line with no antibody. Maximal lysis=cell line+Triton X-100. Cytotoxicity assessed using the CytoTox-Glo™ Cytotoxicity Luminescence assay (Promega).

AB79 does not have Agonist Activity

The capacity of AB79 treatment to induce cytokine production in human PBMCs was compared to negative IgG1 isotype control and positive controls, PHA, anti-CD3 (clone OKT3) or anti-CD52 (Campath) antibodies.

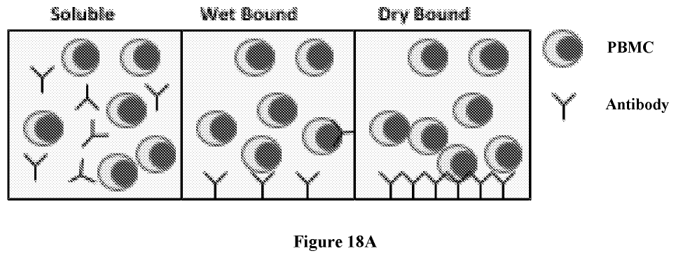

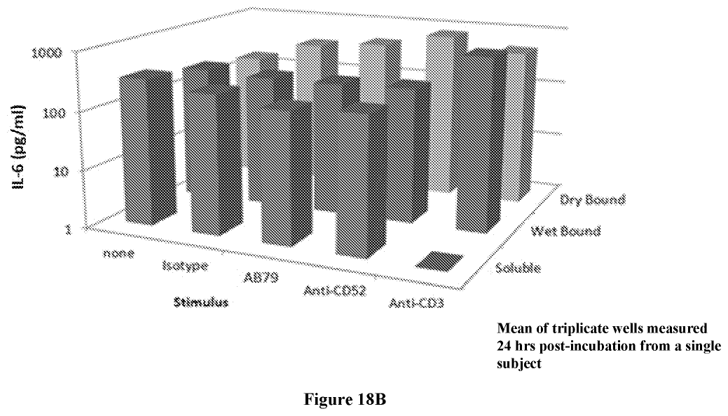

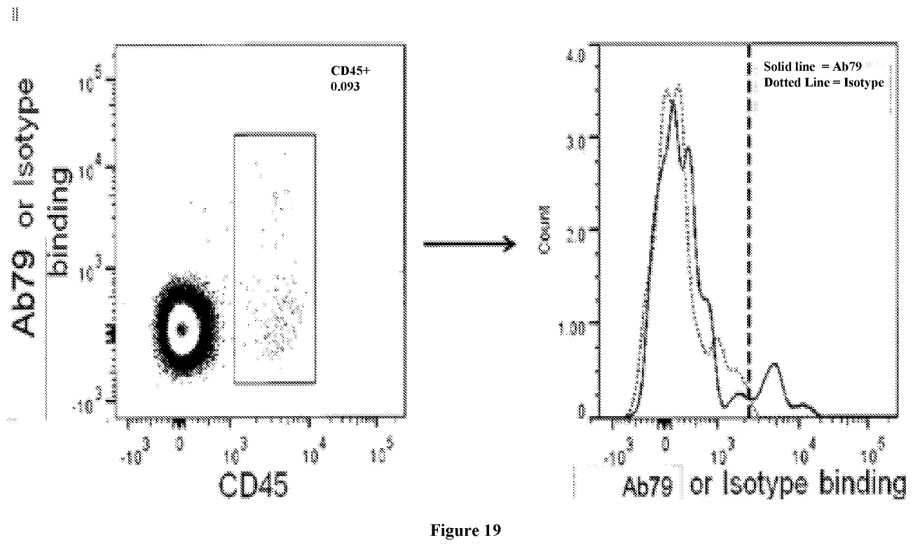

Soluble AB79 did not increase IL-6 levels (mean SD) in PBMCs collected from 4 different subjects after a 24 hour incubation as compared to IgG1 isotype control. PHA increased cytokine levels in all subjects demonstrating that the cells had the capacity to make IL-6. Similar results were seen with PBMCs stimulated for 48 hours and when IL-2, IL-4, IL-10, GM-CSF, IFNγ and TNFα were tested (data not shown) (FIG. 18).

The method by which an antibody is presented to a cell may contribute to the outcome of antibody: ligand engagement and cell response (Stebbings et al. (2007) J. Immunol. 179: 3325-3331). Stebbings et al. showed that the maximal cell response (cytokine release) to an agonistic antibody occurred when the antibody was highly concentrated and adhered to the well surface such as when antibody was added to a well in solution and the liquid allowed to evaporate (Dry Bound) as compared to antibodies allowed to bind to wells in solution (Wet Bound) or added directly to PBMCs (Soluble) (FIG. 18A). AB79 did not stimulate cytokine production in using any of these approaches (FIG. 18B).

AB79 (100 mg/ml) did not stimulate IL-2, -4, -6, -8, -10, GM-CSF, IFN or TNFα under any of the conditions tested after 24 hours. AB79 did not induce IL-10 or GM-CSF, but both were induced by anti-CD3 (not shown, all values except anti-CD3 were below LLOQ). IL-8 was constitutively produced by PBMCs and was not altered by any treatment (data not shown) (Table 6).

TABLE 6
AB79 and Cytokine Stimulation

| | | | | | | |
|---|---|---|---|---|---|---|
| Antibody | | | | | | |
| Presentation | None | Isotype | AB79 | Anti-CD52 | PHA | Anti-CD3 |

IL-2

| | | | | | | |
|---|---|---|---|---|---|---|
| Soluble | LLOQ | LLOQ | LLOQ | LLOQ | 14953 ± 3117 | nd |
| Wet Bound | LLOQ | LLOQ | LLOQ | LLOQ | Nd | 395.0 ± 64.8 |
| Dry Bound | LLOQ | LLOQ | LLOQ | LLOQ | Nd | 167.9 ± 90.1 |

IL-4

| | | | | | | |
|---|---|---|---|---|---|---|
| Soluble | 7.3 ± 1.4 | 4.1 ± 0.8 | 6.4 ± 3.2 | 6.9 ± 4.0 | 50.4 ± 8.9 | nd |
| Wet Bound | 5.3 ± .08 | 3.8 ± 1.5 | 6.4 ± 0.6 | 8.5 ± 3.7 | Nd | 17.8 ± 2.4 |
| Dry Bound | 4.4 ± 1.8 | 7.8 ± 2.7 | 9.1 ± 2.6 | 10.5 ± 2.1 | Nd | 16.2 ± 3.1 |

IL-6

| | | | | | | |
|---|---|---|---|---|---|---|
| Soluble | 325.1 ± 65.3 | 236.0 ± 98.9 | 170.5 | 202.0 ± 48.0 | 18880 ± 0 | nd |
| Wet Bound | 216.8 ± 95.1 | 191.2 ± 47.0 | 194.6 ± 66.0 | 207.2 ± 60.2 | Nd | 902.7 ± 114.1 |
| Dry Bound | 165.0 ± 79.4 | 369 ± 143 | 465.8 ± 230 | 811.1 ± 473.7 | Nd | 500 ± 17 |

IFNγ

| | | | | | | |
|---|---|---|---|---|---|---|
| Soluble | 1190.2 ± 117.5 | 857.1 ± 311.7 | 770.1 ± 203.6 | 1116.9 ± 330.8 | 15857 ± 4614.8 | nd |
| Wet Bound | 1052.8 ± 385.8 | 657.2 ± 222.6 | 993.4 ± 198.1 | 1138.9 ± 339.1 | Nd | 5674.6 ± 564.7 |
| Dry Bound | 768.1 ± 110.0 | 1583.1 ± 418.6 | 1927.6 ± 517.6 | 1827.0 ± 281.5 | Nd | 3513.2 ± 708.3 |

TNFα

| | | | | | | |
|---|---|---|---|---|---|---|
| Soluble | 28.71 ± 8.1 | 11.6 ± 7.1 | 59.6 ± 90.1 | 79.9 ± 18.2 | 9270.0 ± 0 | nd |
| Wet Bound | 16.5 ± 3.4 | 16.1 ± 3.9 | 14.8 ± 8.8 | 166.1 ± 21.7 | Nd | 2123.3 ± 239.7 |
| Dry Bound | 16.2 ± 7.4 | 919.2 ± 77.4 | 745.1 ± 141 | 984.4 ± 317.0 | Nd | 2231.6 ± 687 |

A Multiplex cytokine assay was used according to manufacturer's instructions (Bio-Plex Pro ™ Human Cytokine Standard 8-Plex) to measure IL-2, -4, -6, -8, -10, GM-CSF, IFNγ and TNFα concentrations. Abbreviations: LLOQ Lower Limit of Quantification; nd, not done; PHA, phytohemagglutinin; PBMC, peripheral blood mononuclear cell.

### AB79 Depletes CD38+ Cells

AB79 binds CD38 with high affinity and mediates CDC and ADCC. AB79 is not an agonist and did not induce cytokine release from human PBMCs. AB79 bound CD38 from both human and cynomolgus monkey. Lymphocytes from both species had similar cell-specific patterns of CD38 expression with NK cells>B cells>T cells based on Median Fluorescent Intensity of AB79 staining. Treatment with AB79 depleted monkey lymphocytes in a reversible, cell-specific and dose-dependent manner. AB79 effectively blocked human antibody recall response in a mouse adoptive transfer model.

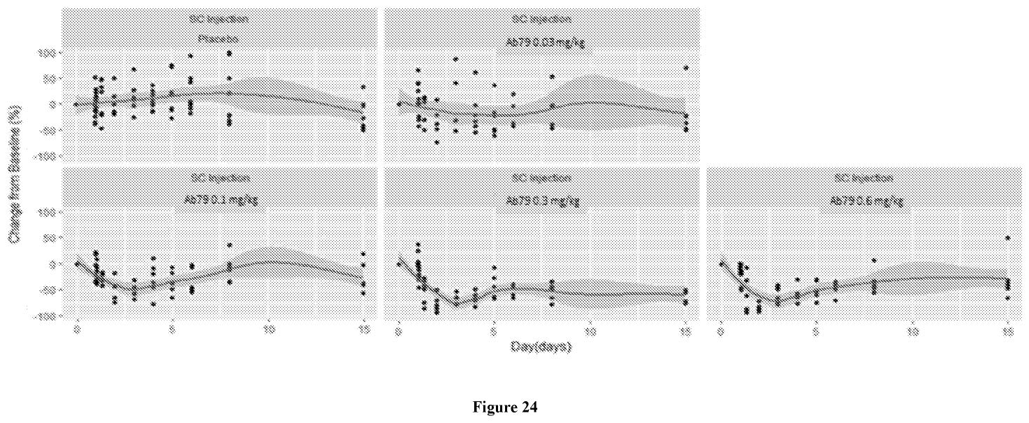

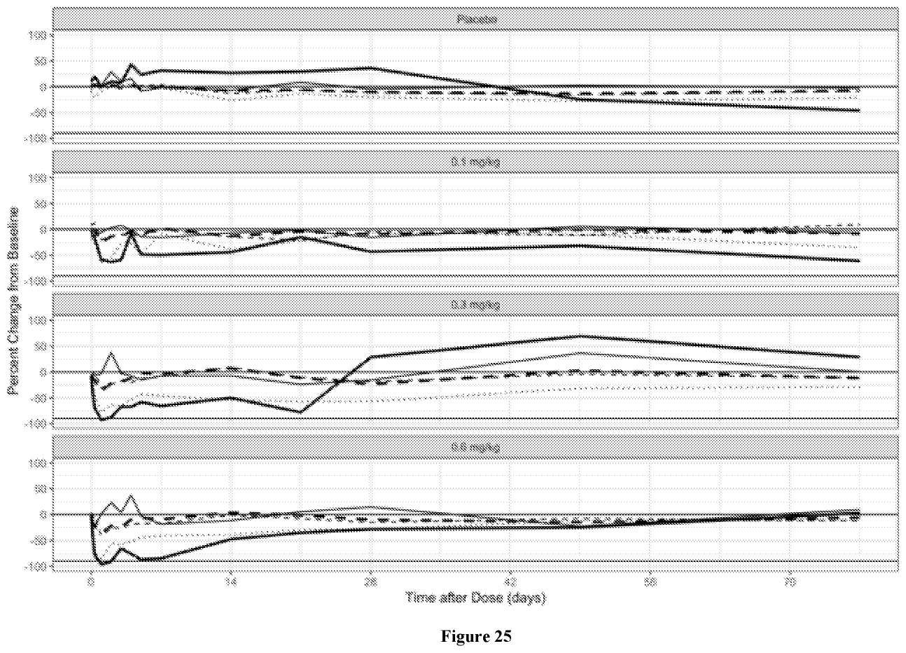

In the cohorts treated with AB79 by SC injection, a dose-dependent reduction in NK cells (FIG. 24) and plasmablasts (FIG. 25) were observed at doses≥0.1 mg kg⁻¹ with ≥90% reduction in plasmablasts within all subjects receiving a 0.6 mg kg⁻¹ injection. A 75% reduction in NK cells occurred at 0.6 mg kg⁻¹ (data not shown) with a Cₘₐₓ of 23.0 ng mL⁻¹ (Table 7). The levels of plasmablasts and NK cells were reduced from baseline within 8 hours after injection and exhibited a tₘₐₓ of 48 hours. The duration of recovery to baseline levels was variable; recovery to baseline (i.e., within −20% of baseline levels) for the 0.1, 0.3, and 0.6 mg kg⁻¹ doses required a mean of 4, 78, and 50 days, respectively (data not shown). There were minimal or no reductions observed for total lymphocytes, B and T cells, cytotoxic T cells, helper T cells, monocytes (FIG. 25) and granulocytes, red blood cells and platelets (data not shown).

TABLE 7
Summary PK Parameters of AB79 Following a Single SC
Injection of AB79 at 0.6 mg kg−1 to Healthy Subjects

| | | | tₘₐₓ | Cₘₐₓ | AUCₗₐₛₜ |
|---|---|---|---|---|---|
| | | | (h) | (ng mL⁻¹) | (ng day⁻¹ mL⁻1) |
| Route | Dose | n | n = 6 | n = 6 | n = 6 |
| SC | 0.6 mg kg⁻¹ | 6 | 23.87 (7.98, 96.02)^{a} | 23.0 (67) | 90.4 (92) |

^{a}n = 5.
Values represent mean (% CV), except for tₘₐₓ where median (min, max) are presented. AUCₗₐₛₜ, area under the serum concentration-time curve from time 0 to time of the last quantifiable concentration; Cmax, maximum observed serum concentration; CV, coefficient of variance; IV, intravenous; NA, not applicable; PK, pharmacokinetics; SC, subcutaneous; tmax, time to maximum serum concentration.

### AB79 and Daratumumab Red Blood Cell Binding Summary

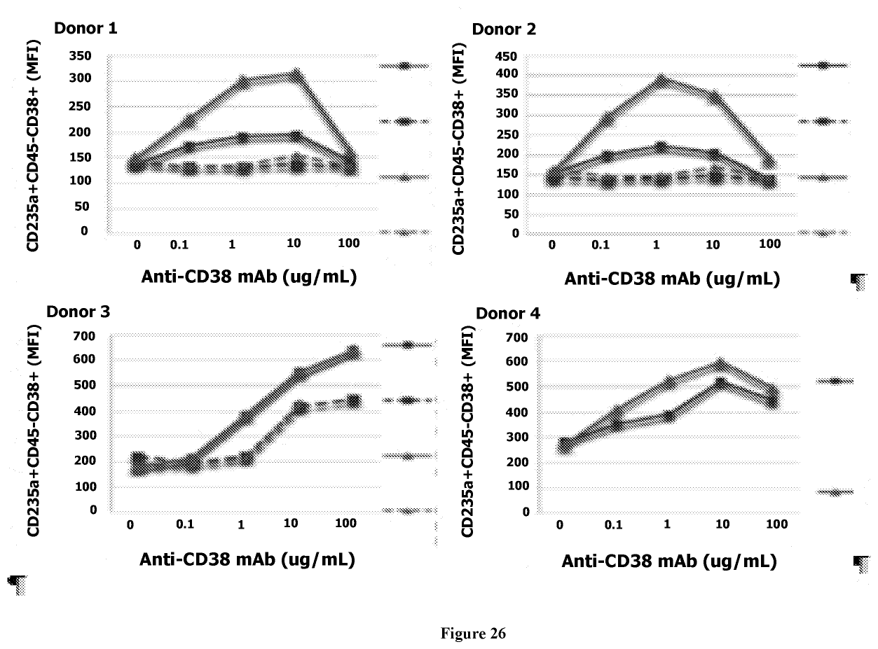

The RBC binding profiles of AB79 and daratumumab were compared. As depicted in FIG. 26, there appeared to be a difference in the magnitude of RBC binding (i.e., MFI) between the drug products in 3 of 4 donors tested; however, this difference may be attributed to the differential biotin levels on each of the antibodies, with daratumumab having 1.6- to 2.0-fold more biotin then AB79. An alternate analysis, which controls for a potential difference in the fluorescent labeling of antibodies, is to compare the concentration versus binding profile of each antibody and a useful metric is the concentration at which the maximum binding occurs (i.e., maximum specific binding of antigen (Bmax)). The Bmax is identical for both antibodies in 3 of 4 donors (e.g., 1 μg/mL for Donor 1). Collectively, these data indicate that both antibodies bind with similar affinities, within the current resolution limit of the assay, which is a factor of 10. In conclusion, both AB79 and daratumumab bound to RBCs in this assay with affinities that were within 10-fold of one another; a 10-fold or greater difference in binding affinity of these antibodies for RBCs did not exist within this assay system.

### Example 3: A Phase 1/2a Study to Investigate the Safety, Tolerability, Efficacy, Pharmacokinetics, and Immunogenicity of AB79 Administered Subcutaneously as a Single Agent in Human Patients with Relapsed/Refractory (r/r) Multiple Myeloma (MM)

The purpose of this study is to assess the safety, tolerability, pharmacokinetics (PK), immunogenicity, dose-limiting toxicity (DLT) and maximum tolerated dose (MTD)/recommended phase 2 dose (RP2D) in Phase 1 of the study and to provide a preliminary evaluation of the clinical activity of AB79 monotherapy in participants, with relapsed and/or refractory multiple myeloma (RRMM). The study includes patients with RRMM who have been previously treated with at least a proteasome inhibitor (PI), an immunomodulatory drug (IMid), an alkylating agent, and asteroid. Patients should have refractory disease or be intolerant to at least 1 PI and at least 1 IMiD, and they should have either received 3 or more prior therapies or received at least 2 prior therapies if one of those therapies included a combination of a PI and an IMiD. In the phase 1b dose-escalation part, previous exposure to an anti-CD38 agent is allowed but not required. In the phase 2a expansion part of the study, patients must also have disease refractory to at least 1 anti-CD38 monoclonal therapy at any time during treatment. The study is a multi-center trial conducted in the United States comprising approximately 42 participants.

### Phase 1

The study population of Phase 1 consists of approximately 24 adult participants, aged 18 years or over. The patient characteristics are shown in Table 8.

TABLE 8
Patient Characteristics

| | 45 mg | 135 mg | 300 mg | 600 mg | Total |
|---|---|---|---|---|---|
| | (n = 4) | (n = 3) | (n = 6) | (n = 6) | (n = 19) |
| Dose Level Characteristics | | | | | |
| Median age, years (range) | 64.5 (53, 75) | 69 (64, 74) | 63.5 (56, 69) | 62.5 (60, 77) | 64 (53, 77) |
| Male, % | 50 | 67 | 67 | 67 | 63 |
| ECOG PS 0/1/2, % | 85/15/0 | 25/75/0 | 33/67/0 | 0/83/17 | 26/68/5 |
| ISS I/II/III, missing % | 50/25/25/0 | 33/33/0/33 | 50/33/17/0 | 17/50/33/0 | 37/37/21/5 |
| Median no. prior lines of | 4.5 (2-6) | 3 (2-6) | 3 (2-8) | 5.5 (3-8) | 4 (2-8) |
| therapy (range) | | | | | |
| Types of Therapy: | | | | | |
| Proteasome inhibitor-based, n | 4 (100) | 3 (100) | 4 (100) | 6 (100) | 19 (100) |
| (%) | | | | | |
| IMID-based, n (%) | 4 (100) | 3 (100) | 4 (100) | 6 (100) | 19 (100) |
| Monoclonal antibody-based, n | 1 (25) | 0 | 3 (50) | 3 (50) | 7 (37) |
| (%) | | | | | |
| Daratumumab-based, n (%) | 1 (25) | 0 | 1 (17) | 3 (50) | 5 (26) |
| ASCT, n (%) | 4 (100) | 3 (100) | 6 (100) | 5 (83) | 18 (95) |
| Refractory to last therapy, n | 3 (75) | 1 (33) | 4 (67) | 4 (67) | 12 (63) |
| (%) | | | | | |

Participants in Phase 1 are assigned to 1 of 6 dose-escalation AB79 treatment groups: Cohort 1: 45 mg; Cohort 2: 135 mg; Cohort 3: 300 mg; Cohort 4: 600 mg; Cohort 5: 1200 mg; and Cohort 6: 1800 mg (Table 9). AB79 is delivered by subcutaneous injection, once weekly for 8 weeks, then once every 2 weeks for 16 weeks, and then once every 4 weeks thereafter in a 28-day treatment cycle until disease progressions (PD), unacceptable toxicities or withdrawal due to other reasons. Participants may receive premedications 1 to 3 hours prior to the administration of AB79 on each dosing day, as follows: for example, Dexamethasone (20 mg); Acetaminophen (650 to 1000 mg); Diphenhydramine (25 to 50 mg); and Montelukast (10 mg).

TABLE 9
Phase 1 Cohorts

| Cohort | Dosage Regimen |
|---|---|
| Cohort 1: | Subcutaneous injection of 45 mg AB79, once weekly |
| AB79 45 mg | for 8 weeks, then once every 2 weeks for 16 weeks, and |
| | then once every 4 weeks thereafter in a 28-day |
| | treatment cycle until PD, unacceptable toxicities or |
| | withdrawal due to other reasons. Dose escalation of |
| | AB79 to 135 mg may be done using a 3 + 3 dose |
| | escalation design to determine a MTD and/or RP2D. |
| Cohort 2: | Subcutaneous injection of 135 mg AB79, once weekly |
| AB79 135 mg | for 8 weeks, then once every 2 weeks for 16 weeks, and |
| | then once every 4 weeks thereafter in a 28-day |
| | treatment cycle until PD, unacceptable toxicities or |
| | withdrawal due to other reasons. Dose escalation of |
| | AB79 to 300 mg may be done using a 3 + 3 dose |
| | escalation design to determine a MTD and/or RP2D. |
| Cohort 3: | Subcutaneous injection of 300 mg AB79, once weekly |
| AB79 300 mg | for 8 weeks, then once every 2 weeks for 16 weeks, and |
| | then once every 4 weeks thereafter in a 28-day |
| | treatment cycle until PD, unacceptable toxicities or |
| | withdrawal due to other reasons. Dose escalation of |
| | AB79 to 600 mg may be done using a 3 + 3 dose |
| | escalation design to determine a MTD and/or RP2D. |
| Cohort 4: | Subcutaneous injection of 600 mg AB79, once weekly |
| AB79 600 mg | for 8 weeks, then once every 2 weeks for 16 weeks, and |
| | then once every 4 weeks thereafter in a 28-day |
| | treatment cycle until PD, unacceptable toxicities or |
| | withdrawal due to other reasons. Dose escalation of |
| | AB79 to 1200 mg may be done using a 3 + 3 dose |
| | escalation design to determine a MTD and/or RP2D. |
| Cohort 5: | Subcutaneous injection of 1200 mg AB79, once weekly |
| AB79 1200 mg | for 8 weeks, then once every 2 weeks for 16 weeks, and |
| | then once every 4 weeks thereafter in a 28-day |
| | treatment cycle until PD, unacceptable toxicities or |
| | withdrawal due to other reasons. Dose escalation of |
| | AB79 to 1800 mg may be done using a 3 + 3 dose |
| | escalation design to determine a MTD and/or RP2D. |
| Cohort 6: | Subcutaneous injection of 1800 mg AB79, once weekly |
| AB79 1800 mg | for 8 weeks, then once every 2 weeks for 16 weeks, and |
| | then once every 4 weeks thereafter in a 28-day |
| | treatment cycle until PD, unacceptable toxicities or |
| | withdrawal due to other reasons. |

The overall time to participate in this study is 36 months (3 years). In Phase 1, participants who stop treatment for any other reason other than PD continue to have progression-free survival (PFS) follow-up at the site every 4 weeks from the last dose of study drug up to 12 months or until PD, death, loss to follow-up, consent withdrawal or study termination. Participants are followed 30 days after last dose of study drug or until the start of subsequent alternative anti-cancer therapy, whichever occurs first, for a follow up assessment.

### Primary Outcome Measures for Phase 1

Primary outcome measures for up to one year include the following:
Number of Participants Reporting one or more Treatment-Emergent Adverse Events (TEAEs)
Number of Participants with Dose-limiting Toxicities (DLTs): DLTs defined as any of the following events: Grade 4 laboratory abnormalities, except those events that are clearly due to extraneous causes; nonhematologic TEAEs of grade greater than or equal to (≥) 3 except grade 3 nausea/vomiting, fatigue lasting less than 72 hours, elevation of alanine aminotransferase (ALT) or aspartate aminotransferase (AST) that resolves to grade less than or equal to (≤) 1 or baseline within 7 days, injection reaction (IR) that responds to symptomatic treatment; Hematologic TEAEs of National Cancer Institute Common Terminology Criteria for Adverse Events (NCI CTCAE) grade≥4, except grade≥3 hemolysis, grade 3 low platelet or higher count with clinically meaningful bleeding; and an incomplete recovery from treatment-related toxicity causing a greater than (>) 2-week delay in the next scheduled injection before the initiation of Cycle 2 will be considered a DLT.
Number of Participants with Grade 3 or Higher TEAEs: AE Grades are evaluated as per NCI CTCAE, version 4.03. Grade 1 scaled as Mild; Grade 2 scaled as Moderate; Grade 3 scaled as severe or medically significant but not immediately life-threatening; Grade 4 scaled as life-threatening consequences; and Grade 5 scaled as death related to AE.
Number of Participants with Serious TEAEs
Number of Participants with TEAEs Leading to Treatment Discontinuation
Number of Participants with TEAEs Leading to Dose Modifications

### Secondary Outcome Measures:

Secondary outcome measures include the following:
Cmax: Maximum Observed Serum Concentration for AB79 [Time Frame: Cycle 1 and 2: Day 1 pre-dose and at multiple time points (up to 168 hours) post-dose.]
Tmax: Time to Reach the Maximum Observed Serum Concentration (Cmax) for AB79 [Time Frame: Cycle 1 and 2: Day 1 pre-dose and at multiple time points (up to 168 hours) post-dose.]
AUC: Area Under the Serum Concentration-time Curve from Time 0 to the Time of the Last Quantifiable Concentration for AB79 [Time Frame: Cycle 1 and 2: Day 1 pre-dose and at multiple time points (up to 168 hours) post-dose.]
Phase 1: ORR [Time Frame: Up to 1 year]: ORR is defined as the percentage of participants who achieved a PR of 50% tumor reduction or better during the study. PR is defined as ≥50% reduction of serum M-protein and reduction in 24-hour urine M-protein by ≥90% or to <200 mg/24 hours.
Percentage of Participants with Minimal Response (MR) [Time Frame: Up to 1 year]: MR is defined as ≥25% but ≤49% reduction of serum M-protein and reduction in 24-hour urine M-protein by 50% to 89%.
Percentage of Participants with Positive Anti-drug Antibodies (ADA) [Time Frame: Up to 1 year].

### Results:

As of 5 Mar. 2019, 19 patients have been enrolled across 4 dosing cohorts (45 mg, 135 mg, 300 mg, and 600 mg) and have received at least 1 cycle of AB79. As of 5 Mar. 2019, 9 patients are still being monitored on treatment, 9 patients have discontinued AB79 due to disease progression, and 1 patient withdrew consent.

As of 5 Mar. 2019, no dose-limiting toxicities (DLTs) have been reported in DLT-evaluable patients across all 4 cohorts (45 mg, 135 mg, 300 mg, and 600 mg). There have not been any injection site reactions or systemic infusion reactions. No drug-related SAEs, on-study deaths, or AEs that led to study discontinuation were reported. (See Table 10). Further, except for 1 transient treatment-related Grade 3 event of decreased neutrophil count and 1 transient anemia, there have not been any remarkable laboratory findings. Disease progression in one patient was associated with Grade 3 decrease in platelet count, anemia, and increased creatinine. This study is ongoing and patients continue to be followed.

TABLE 10
Summary of TEAEs

| As of 9 Jan. 2019 | 45 mg | 135 mg | 300 mg | 600 mg | Total | |
|---|---|---|---|---|---|---|
| N (%) | (n = 4) | (n = 3) | (n = 6) | (n = 6) | (n = 19) | |
| Any TEAE | 4 (100) | 3 (100) | 6 (100) | 5 (83) | 18 | (95) |
| Any drug-related | 3 (75) | 2 (67) | 3 (50) | 3 (50) | 11 | (58) |
| TEAE | | | | | | |
| Grade 3 or higher | 2 (50) | 1 (33) | 1 (17) | 2 (33) | 6 | (32) |
| TEAE | | | | | | |
| Drug-related | 1 (25) | 0 | 1 (17) | 0 | 2 | (11) |
| Grade 3 or higher | | | | | | |
| TEAE | | | | | | |
| SAE | 1 | 0 | 0 | 0 | 1 | (5) |

| | | | | | |
|---|---|---|---|---|---|
| Drug-related | 0 | 0 | 0 | 0 | 0 |
| SAE | | | | | |
| Discontinuation | 0 | 0 | 0 | 0 | 0 |
| due to TEAE | | | | | |
| Dose Limiting | 0 | 0 | 0 | 0 | 0 |
| Toxicity | | | | | |
| On-Study Death | 0 | 0 | 0 | 0 | 0 |

As of 5 Mar. 2019, the most common (in >2 patients) TEAEs by Preferred Terms regardless of causality across all 4 cohorts are anemia (n=7 patients), insomnia (n=5 patients each), upper respiratory tract infection, dizziness, headache, and hypertension (n=4 patients each), and diarrhea, fatigue, decreased appetite, and muscle spasms (n=3 patients each). All AEs have been Grade 1 or 2 except for anemia (n=2 patients), diarrhea, decrease platelet count, decrease neutrophil count, increased creatinine, headache, hypertension, and musculosketal pain (n=1 patient each) which were grade 3; only 1 event of low neutrophil count and 1 event of anemia was reported as related to study drug. No cases of IRRs, cytokine release syndrome, or injection site reactions have been observed. Therefore, the present invention provides a safe anti-CD38 antibody for clinical application as compared with previous anti-CD38 antibodies, such as daratumumab, isatuximab or MOR202.

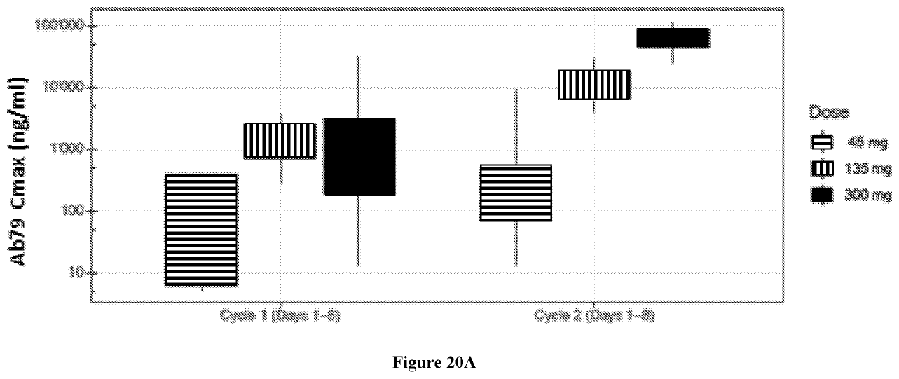

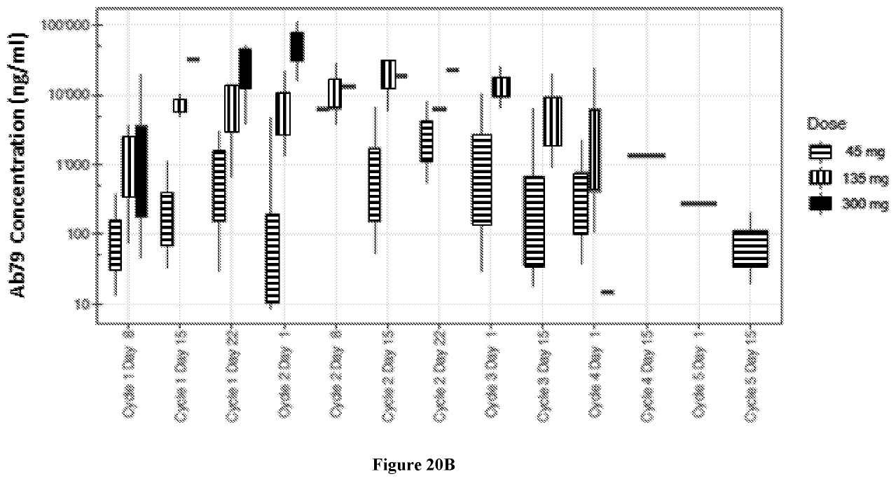

FIG. 20 shows that the subcutaneously administered Ab79 exposure increased with increasing the doses over time, which is consistent with target-mediated drug clearance.

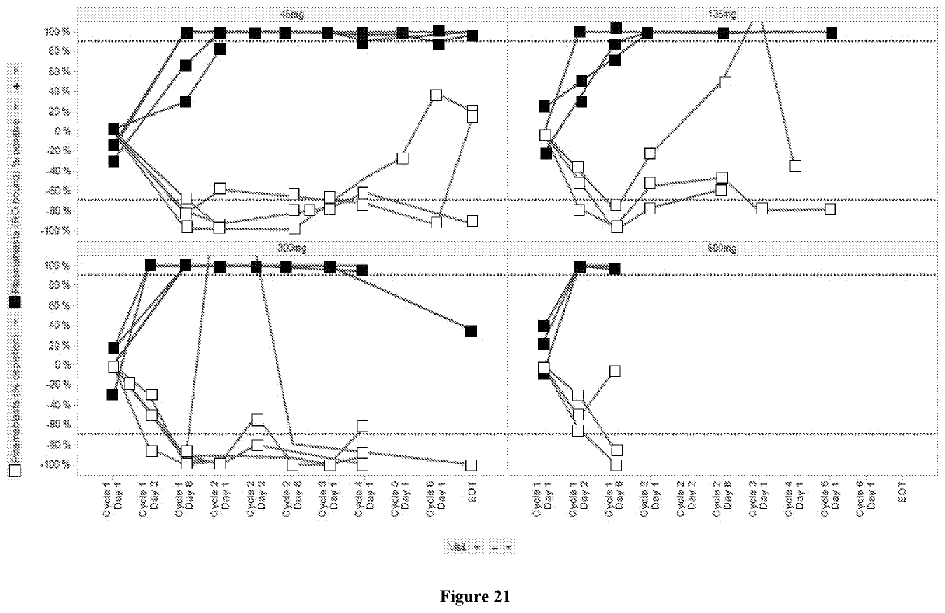

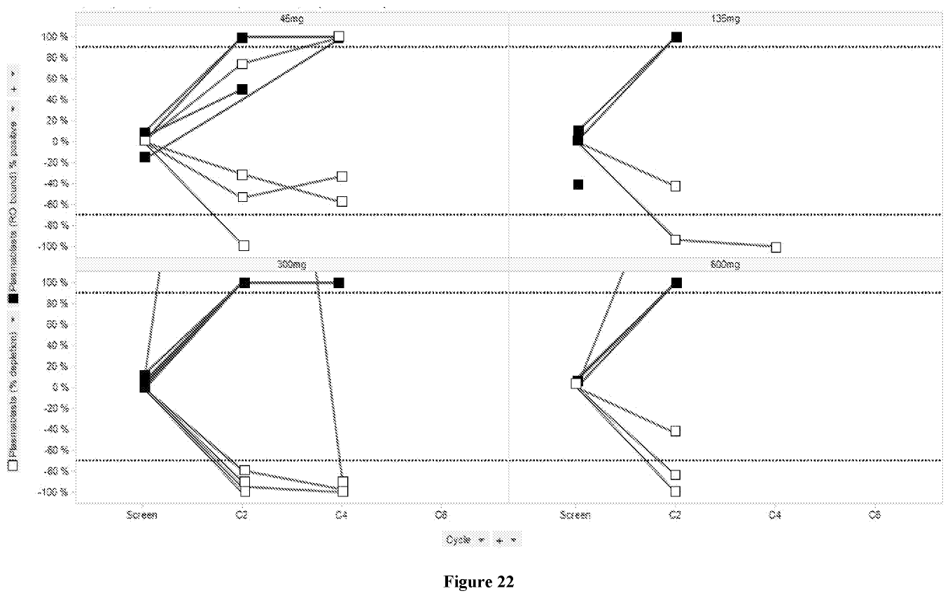

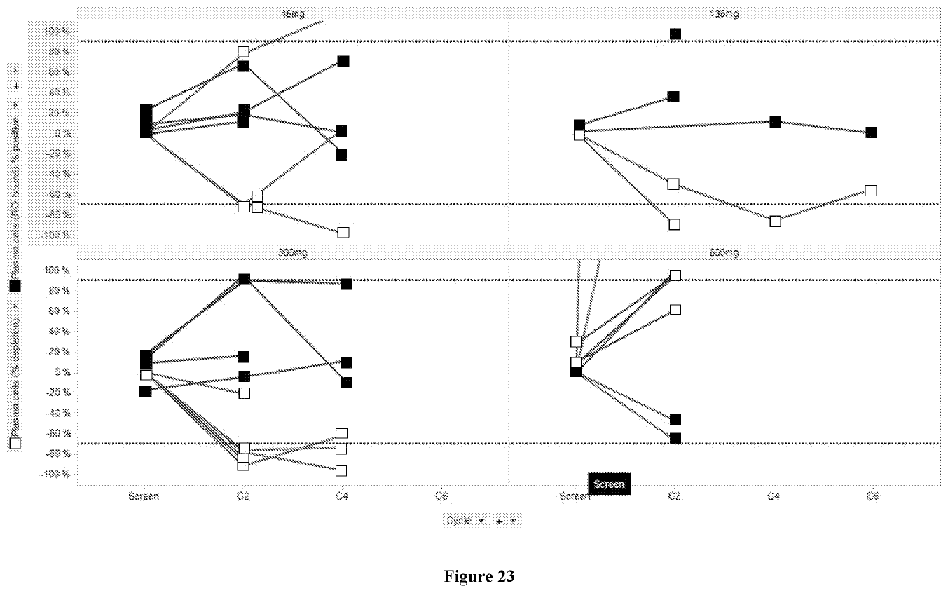

Subcutaneously administered Ab79 reduced levels of plasmablasts in blood (FIG. 21), plasmablasts in bone marrow aspirates (FIG. 22), and plasma cells in bone marrow aspirates (FIG. 23) in a dose dependent manner. CD38 was saturated on target cells in peripheral blood at doses≥45 mg weekly and in bone marrow≥300 mg. Levels of target cells in bone marrow and peripheral blood were reduced in a dose-dependentmanner at doses≤300 mg.

In patients with advanced RRMM, AB79 has shown early signs of anti-tumor activity as evidenced by at least 50% reduction in disease burden in some patients and prolonged disease stabilization in others. Though additional data are needed to characterize the clinical benefit of this drug, the emerging data supports the ongoing development of AB79.

Two CD38 mAbs are currently in clinical development: intravenous daratumumab is approved for patients with MM (relapsed and newly diagnosed), and intravenous isatuximab is investigational. The most frequent adverse reactions (≥20%) with daratumumab monotherapy or in combination with standard anti-myeloma regimens are infusion-related reactions (IRRs), neutropenia, thrombocytopenia, fatigue, nausea, diarrhea, constipation, vomiting, muscle spasms, arthralgia, back pain, pyrexia, chills, dizziness, insomnia, cough, dyspnea, peripheral edema, peripheral sensory neuropathy, and upper respiratory tract infections. (Darzalex USPI). Daratumumab can cause severe and/or serious infusion reactions including anaphylactic reactions and have been reported in approximately half of all patients (Darzalex USPI). Attention must also be paid to daratumumab interference with certain laboratory assays, which importantly may complicate blood compatibility testing. (Darzalex USPI). Isatuximab, a humanized anti-CD38 monoclonal antibody, is also being investigated in multiple myeloma. Reported AEs for isatuximab (≥24%) include infusion reactions, nausea, fatigue, dyspnea, and cough, which were typically grade≤2 (Richter et al. (2016) JCO 34 (15): 8005; Dimopoulos et al. (2018) Blood 132 (suppl. 1): ASH abstract 155/oral presentation)). Therefore, based on the available evidence, there remains a need for new agents, including a new generation of CD-38 targeted therapy with greater selectivity thus more potency, resulting in less toxicity, and improved patient convenience to continue to improve clinical outcomes.

### Phase 2

The study population of Phase 2a consists of approximately 18 participants. Dose and premedications for Phase 2a are based upon review of the available safety, efficacy, PK, and pharmacodynamic data from the preceding cohorts of Phase 1.

TABLE 11
Phase 2 Cohorts

| Cohort | Dosage Regimen |
|---|---|
| Cohort 1: | Subcutaneous injection of AB79, once weekly for 8 |
| AB79 TBD | weeks, then once every 2 weeks for 16 weeks, and then |
| | once every 4 weeks thereafter in a 28-day treatment |
| | cycle until PD, unacceptable toxicities or withdrawal |
| | due to other reasons. AB79 dose for this phase is |
| | determined based on review of the available safety, |
| | efficacy, PK, and pharmacodynamic data obtained from |
| | the Phase 1 portion of the study. |

### Primary Outcome Measures for Phase 2a

Primary outcome measures for up to one year include the following:
Overall Response Rate (ORR): ORR is defined as the percentage of participants who achieved a partial response (PR) of 50 percent (%) tumor reduction or better during the study. PR is defined as ≥50% reduction of serum M-protein and reduction in 24-hour urine M-protein by ≥90% or to less than (<) 200 milligram per (mg/) 24 hours.

### Secondary Outcome Measures for Phase 2a

Secondary outcome measures for up to one year include the following:
Phase 2a: Number of Participants with DLTs
Phase 2a: Number of Participants Reporting one or more TEAEs
Phase 2a: Number of Participants with TEAEs Leading to Dose Modifications
Phase 2a: Number of Participants with TEAEs Leading to Treatment Discontinuation
Phase 2a: Number of Participants with Clinically Significant Laboratory Values
Phase 2a: Number of Participants with Clinically Significant Vital Sign Measurements
Phase 2a: Duration of Response (DOR): DOR is the time from the date of first documentation of response to the date of first documented PD. PD is the increase of ≥25% from lowest response value in any of the following: Serum M-protein (increase must be ≥0.5 g/dL; serum M component increases ≥1 g/dL are sufficient to define relapse if starting M component is ≥5 g/dL),and/or urine M-protein (increase must be ≥200 mg/24 hour), and/or only in participants without measurable serum and/or urine M-protein levels, difference between involved/uninvolved free light chain (FLC) levels (increase must be >10 mg/dL), and only in participants without measurable serum and/or urine M-protein levels and without measurable disease by FLC levels, bone marrow plasma cell percentage (percentage must be ≥10%) or definite development of new bone lesions or soft tissue plasmacytomas or increase in size of bone lesions or soft tissue plasmacytomas, and development of hypercalcemia that can be attributed solely to plasma cell proliferative disorder.
Phase 2a: Progression Free Survival (PFS): PFS is the time from the date of the first dose until the earliest date of PD. PD is the increase of ≥25% from lowest response value in any of the following: Serum M-protein (increase must be ≥0.5 g/dL; serum M component increases ≥1 g/dL are sufficient to define relapse if starting M component is ≥5 g/dL), and/or urine M-protein (increase must be ≥200 mg/24 hour), and/or only in participants without measurable serum and/or urine M-protein levels, difference between involved/uninvolved FLC levels (increase must be >10 mg/dL), and only in participants without measurable serum and/or urine M-protein levels and without measurable disease by FLC levels, bone marrow plasma cell percentage (percentage must be ≥10%) or definite development of new bone lesions or soft tissue plasmacytomas or increase in the size of bone lesions or soft tissue plasmacytomas, and development of hypercalcemia that can be attributed solely to plasma cell proliferative disorder.
Phase 2a: Overall Survival (OS): OS is defined as the time from the date of first dose to the date of death due to any cause.
Phase 2a: Time to Response (TTR): TTR is defined as the time from the date of the first dose to the date of the first documentation of response (partial response (PR) or better). PR is defined as ≥50% reduction of serum M-protein and/or reduction in 24-hour urine M-protein by ≥90% or to <200 mg/24 hours.

### Inclusion Criteria for Phase 1 and Phase 2a Study:

Subjects have received the final dose of any of the following treatments/procedures within the specified minimum intervals before the first dose of AB79: Myeloma-specific therapy (washout period of 30 days); antibody therapy (including anti-CD38) (washout period of 120 days); corticosteroid therapy (washout period of 30 days); autologous transplantation (washout period of 90 days); radiation therapy (washout period of 30 days); major surgery (washout period of 30 days).

For Participants with MM, measurable disease defined as one of the following: (a) Serum M-protein≥500 mg/dL (≥5 g/L); (b) Urine M-protein≥200 mg/24 hours; (c) For participants without measurable M-protein in serum protein electrophoresis (SPEP) or urine protein electrophoresis (UPEP), a serum FLC assay result with involved FLC level≥10 mg/dL (≥100 mg/L), provided serum FLC ratio is abnormal.

Prior therapy should meet all of the following criteria: (a) participant previously treated with at least a proteasome inhibitor (PI), an immunomodulatory drug (IMid), an alkylating agent, and a steroid; (b) participant refractory or intolerant to at least 1 PI and at least 1 IMid; patient either has received ≥3 prior lines of therapy or has received at least 2 prior lines of therapy if one of those lines included a combination of PI and IMid; (c) participant can have had previous exposure to an anti-CD38 agent, as a single agent or in combination, but this is not required.

In the phase 2a portion of the study, participants with MM must also have been refractory to at least 1 anti-CD38 monoclonal antibody therapy at any time during treatment. “Refractory” is defined as at least a 25% increase in M-protein or PD during treatment or within 60 days after cessation of treatment. “Line of therapy” is defined as 1 or more cycles of a planned treatment program. This may consist of 1 or more planned cycles of single-agent therapy or combination therapy, as well as a sequence of treatments administered in a planned manner. A new line of therapy starts when a planned course of therapy is modified to include other treatment agents (alone or in combination) as a result of PD, relapse, or toxicity. A new line of therapy also starts when a planned period of observation off therapy is interrupted by a need for additional treatment for the disease.

### Exclusion Criteria for the Study:

1. Sensory or motor neuropathy of National Cancer Institute Common Terminology Criteria for Adverse Events (NCI CTCAE) Grade≥3.

2. Have received allogeneic stem cell transplant.

3. Have received anti-CD38 antibody therapy and do not fulfill a 120-day washout period before receiving AB79.

4. Not recovered from adverse reactions to prior myeloma treatment or procedures (chemotherapy, immunotherapy, radiation therapy) to NCI CTCAE Grade≤1 or baseline.

5. Clinical signs of central nervous system (CNS) involvement of MM.

6. Active chronic hepatitis B virus (HBV) or hepatitis C virus (HCV) infection, active HIV, or cytomegalovirus (CMV) infection.

7. POEMS (Polyneuropathy, organomegaly, endocrinopathy, monoclonal gammopathy and skin changes) syndrome, monoclonal gammopathy of unknown significance, smoldering myeloma, solitary plasmacytoma, amyloidosis, Waldenström macroglobulinemia, or IgM myeloma.

8. Have positive Coombs tests at screening.

### INCORPORATION BY REFERENCE

The contents of all cited references (including literature references, patents, patent applications, and websites) that may be cited throughout this application are hereby expressly incorporated by reference in their entirety for any purpose, as are the references cited therein, to the same extent as if each individual reference was specifically and individually indicated to be incorporated by reference in its entirety for any purposes.

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein. Modifications for carrying out the invention that are obvious to persons of skill in the art are intended to be within the scope of the appended claims.

We claim:

1. A method for treating a disease in a subject, the method comprising subcutaneously administering to the subject an isolated human anti-CD38 antibody sufficient, wherein the anti-CD38 antibody comprises a variable heavy (VH) chain region comprising a CDR1 having the amino acid sequence of SEQ ID NO:3, a CDR2 having the amino acid sequence of SEQ ID NO:4, and a CDR3 having the amino acid sequence of SEQ ID NO:5; and a variable light (VL) chain region comprising a CDR1 having the amino acid sequence of SEQ ID NO:6, a CDR2 having the amino acid sequence of SEQ ID NO:7 and a CDR3 having the amino acid sequence of SEQ ID NO:8, wherein the disease is one for which binding to CD38 is indicated, and wherein the antibody is administered in a dosage of from 45 to 1,800 milligrams.

2. The method of claim 1, wherein the administering of the anti-CD38 antibody does not cause hemolytic anemia or thrombocytopenia.

3. The method of any one of the preceding claims, wherein administering the anti-CD38 antibody results in less than 30% incidence of grade 3 or 4 of one or more treatment-related adverse events (TRAEs) or treatment-emergent adverse events (TEAEs) selected from the group consisting of anemia, hemolytic anemia, thrombocytopenia, fatigue, infusion-related reactions (IRRs), leukopenia, and lymphopenia.

4. The method of anyone of the preceding claims, wherein the anti-CD38 antibody results in less than 10%, less than 9%, less than 8%, less than 7%, less than 6%, less than 5%, less than 4%, less than 3%, less than 2%, less than 1% depletion of RBCs.

5. The method of any one of the preceding claims, wherein the anti-CD38 antibody results in less than 10%, less than 9%, less than 8%, less than 7%, less than 6%, less than 5%, less than 4%, less than 3%, less than 2%, less than 1% depletion of platelets.

6. The method of any one of the preceding claims, wherein the disease is an autoimmune disease or a cancer.

7. The method of any one of the preceding claims, wherein the disease is selected from the group consisting of systemic lupus erythematosus (SLE), rheumatoid arthritis (RA), inflammatory bowel disease (IBD), ulcerative colitis, systemic light chain amyloidosis, and graft-v-host disease.

8. The method of any one of the preceding claims, wherein the disease is selected from the group consisting of multiple myeloma, chronic lymphoblastic leukemia, chronic lymphocytic leukemia, plasma cell leukemia, acute myeloid leukemia, chronic myeloid leukemia, B-cell lymphoma, and Burkitt lymphoma.

9. The method of any one of the preceding claims, wherein the disease is multiple myeloma.

10. The method of any one of the preceding claims, wherein the VH chain region has the amino acid sequence of SEQ ID NO:9 and the VL chain region has the amino acid sequence of SEQ ID NO:10.

11. The method of any one of the preceding claims, wherein the anti-CD38 antibody comprises a heavy chain amino acid sequence of SEQ ID NO:11 and a light chain amino acid sequence of SEQ ID NO:12.

12. The method of any one of the preceding claims, wherein the antibody is administered in a dosage of from 135 to 1,800 milligrams, from 600 to 1,800 milligrams, from 1,200 to 1,800 milligrams, from 45 to 1,200 milligrams, from 45 to 600 milligrams, from 45 to 135 milligrams, from 135 to 1,200 milligrams, from 135 to 600 milligrams, or from 1,200 to 1,800 milligrams.

13. The method of any one of the preceding claims, wherein the human anti-CD38 antibody is administered in the form of a pharmaceutically acceptable composition.

14. The method of any one of the preceding claims, wherein the dosage is a weekly dosage.

15. A method for treating a hematological cancer in a subject, the method comprising subcutaneously administering to the subject an isolated human anti-CD38 antibody, wherein the anti-CD38 antibody comprises a variable heavy (VH) chain region comprising a CDR1 having the amino acid sequence of SEQ ID NO:3, a CDR2 having the amino acid sequence of SEQ ID NO:4, and a CDR3 having the amino acid sequence of SEQ ID NO:5; and a variable light (VL) chain region comprising a CDR1 having the amino acid sequence of SEQ ID NO:6, a CDR2 having the amino acid sequence of SEQ ID NO:7 and a CDR3 having the amino acid sequence of SEQ ID NO:8, and wherein the antibody is administered in a dosage of from 45 to 1,800 milligrams.

16. The method of claim 15, wherein the anti-CD38 antibody does not cause hemolytic anemia or thrombocytopenia.

17. The method of claim 15 or 16, wherein administering the anti-CD38 antibody results in less than 30% incidence of grade 3 or 4 of one or more treatment-related adverse events (TRAEs) or treatment-emergent adverse events (TEAEs) selected from the group consisting of anemia, including hemolytic anemia, thrombocytopenia, fatigue, infusion-related reactions (IRRs), leukopenia, and lymphopenia.

18. The method of any one of claims 15 to 17, wherein the anti-CD38 antibody results in less than 10% depletion of RBCs.

19. The method of any one of claims 15 to 18, wherein the anti-CD38 antibody results in less than 10% depletion of platelets.

20. The method of any one of claims 15 to 19, wherein the hematological cancer is selected from the group consisting of multiple myeloma, chronic lymphoblastic leukemia, chronic lymphocytic leukemia, plasma cell leukemia, acute myeloid leukemia, chronic myeloid leukemia, B-cell lymphoma, and Burkitt lymphoma.

21. The method of claim 20, wherein the hematological cancer is multiple myeloma.

22. The method of any one of claims 15 to 21, wherein the VH chain region has the amino acid sequence of SEQ ID NO:9 and the VL chain region of has the amino acid sequence of SEQ ID NO:10.

23. The method of any one of claims 15 to 22, wherein the anti-CD38 antibody comprises a heavy chain amino acid sequence of SEQ ID NO:11 and a light chain amino acid sequence of SEQ ID NO:12.

24. The method of any one of claims 15 to 23, wherein the antibody is administered in a dosage of from 135 to 1,800 milligrams, from 600 to 1,800 milligrams, from 1,200 to 1,800 milligrams, from 45 to 1,200 milligrams, from 45 to 600 milligrams, from 45 to 135 milligrams, from 135 to 1,200 milligrams, from 135 to 600 milligrams, or from 1,200 to 1,800 milligrams.

25. The method of any one of claims 15 to 24, wherein the human anti-CD38 antibody is administered in the form of a pharmaceutically acceptable composition.

26. The method of any one of claims 15 to 25, wherein the dosage is a weekly dosage.

27. A unit dosage form comprising an isolated antibody that comprises a heavy chain variable region comprising SEQ ID NO:9 and a light chain variable region comprising SEQ ID NO:10, wherein the isolated antibody binds to CD38 and does not bind to human red blood cells, and the unit dosage form is formulated for subcutaneous administration of the antibody at a dosage of from 45 to 1,800 milligrams.

28. The unit dosage form of claim 27, wherein the unit dosage form is formulated for subcutaneous administration of the antibody at a dosage of from 135 to 1,800 milligrams, from 600 to 1,800 milligrams, from 1,200 to 1,800 milligrams, from 45 to 1,200 milligrams, from 45 to 600 milligrams, from 45 to 135 milligrams, from 135 to 1,200 milligrams, from 135 to 600 milligrams, or from 1,200 to 1,800 milligrams.

29. The unit dosage form of claim 27 or claim 28, wherein isolated antibody comprises a heavy chain comprising SEQ ID NO:11 and a light chain comprising SEQ ID NO:12.

30. The unit dosage form of any one of claims 27 to 29, wherein the unit dosage form is formulated for subcutaneous administration of the antibody in the treatment of a hematological cancer selected from the group consisting of multiple myeloma, chronic lymphoblastic leukemia, chronic lymphocytic leukemia, plasma cell leukemia, acute myeloid leukemia, chronic myeloid leukemia, B-cell lymphoma, and Burkitt lymphoma.

31. The unit dosage form of claim 30, wherein the hematological cancer is multiple myeloma.

32. The unit dosage form of any one of claims 27 to 31, wherein the anti-CD38 antibody does not cause hemolytic anemia or thrombocytopenia.

33. The unit dosage form of any one of claims 27 to 32, wherein the anti-CD38 antibody results in less than 10%, less than 9%, less than 8%, less than 7%, less than 6%, less than 5%, less than 4%, less than 3%, less than 2%, less than 1% depletion of RBCs.

34. The unit dosage form of any one of claims 27 to 33, wherein the anti-CD38 antibody results in less than 10%, less than 9%, less than 8%, less than 7%, less than 6%, less than 5%, less than 4%, less than 3%, less than 2%, less than 1% depletion of platelets.

35. The unit dosage form of any one of claim 27 to 34, wherein the dosage is a weekly dosage.


SEQUENCE LISTING (13 sequences)

SEQ ID NO: 1 - protein, 300 aa, Artificial Sequence
  Human CD38
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 301 aa, Artificial Sequence
  Cyno CD38
MANCEFSPVSGDKPCCRLSRRAQVCLGVCLLVLLILVVVVAVVLPRWRQQWSGSGTTSRFPETVLARCVKYTEVHPEMRHVDCQSVWDAFKGAFISKYPCNITEEDYQPLVKLGTQTVPCNKTLLWSRIKDLAHQFTQVQRDMFTLEDMLLGYLADDLTWCGEFNTFEINYQSCPDWRKDCSNNPVSVFWKTVSRRFAETACGVVHVMLNGSRSKIFDKNSTFGSVEVHNLQPEKVQALEAWVIHGGREDSRDLCQDPTIKELESIISKRNIRFFCKNIYRPDKFLQCVKNPEDSSCLSGI

SEQ ID NO: 3 - protein, 8 aa, Artificial Sequence
  HCDR1 AB79
GFTFDDYG

SEQ ID NO: 4 - protein, 8 aa, Artificial Sequence
  HCDR2 AB79
ISWNGGKT

SEQ ID NO: 5 - protein, 16 aa, Artificial Sequence
  HCDR3 AB79
ARGSLFHDSSGFYFGH

SEQ ID NO: 6 - protein, 8 aa, Artificial Sequence
  LCDR1 AB79
SSNIGDNY

SEQ ID NO: 7 - protein, 3 aa, Artificial Sequence
  LCDR2 AB79
RDS

SEQ ID NO: 8 - protein, 10 aa, Artificial Sequence
  LCDR3 AB79
QSYDSSLSGS

SEQ ID NO: 9 - protein, 135 aa, Artificial Sequence
  (VH) chain amino acid sequence
EVQLLESGGGLVQPGGSLRLSCAASGFTFDDYGMSWVRQAPGKGLEWVSDISWNGGKTHYVDSVKGQFTISRDNSKNTLYLQMNSLRAEDTAVYYCARGSLFHDSSGFYFGHWGQGTLVTVSSASTKGPSVFPLA

SEQ ID NO: 10 - protein, 129 aa, Artificial Sequence
  (VL) chain amino acid sequence
QSVLTQPPSASGTPGQRVTISCSGSSSNIGDNYVSWYQQLPGTAPKLLIYRDSQRPSGVPDRFSGSKSGTSASLAISGLRSEDEADYYCQSYDSSLSGSVFGGGTKLTVLGQPKANPTVTLFPPSSEEL

SEQ ID NO: 11 - protein, 453 aa, Artificial Sequence
  heavy chain (HC) amino acid
EVQLLESGGGLVQPGGSLRLSCAASGFTFDDYGMSWVRQAPGKGLEWVSDISWNGGKTHYVDSVKGQFTISRDNSKNTLYLQMNSLRAEDTAVYYCARGSLFHDSSGFYFGHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 216 aa, Artificial Sequence
  light chain (LC) amino acid
QSVLTQPPSASGTPGQRVTISCSGSSSNIGDNYVSWYQQLPGTAPKLLIYRDSQRPSGVPDRFSGSKSGTSASLAISGLRSEDEADYYCQSYDSSLSGSVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 13 - protein, 318 aa, Artificial Sequence
  Human CD157
MAAQGCAASRLLQLLLQLLLLLLLLAAGGARARWRGEGTSAHLRDIFLGRCAEYRALLSPEQRNKNCTAIWEAFKVALDKDPCSVLPSDYDLFINLSRHSIPRDKSLFWENSHLLVNSFADNTRRFMPLSDVLYGRVADFLSWCRQKNDSGLDYQSCPTSEDCENNPVDSFWKRASIQYSKDSSGVIHVMLNGSEPTGAYPIKGFFADYEIPNLQKEKITRIEIWVMHEIGGPNVESCGEGSMKVLEKRLKDMGFQYSCINDYRPVKLLQCVDHSTHPDCALKSAAAATQRKAPSLYTEQRAGLIIPLFLVLASRTQL